(19) United States
(12) Patent Application Publication
(10) Pub. No.: US 2009/0005268 A1
(43) Pub. Date: Jan. 1, 2009

(54) Compositions and Methods for Cancer Diagnostics Comprising Pan-Cancer Markers

(75) Inventor: Kurt Berlin, Stahnsdorf (DE)

Correspondence Address: DAVIS WRIGHT TREMAINE, LLP/Seattle, 1201 Third Avenue, Suite 2200, SEATTLE, WA, 98101-3045, US

(73) Assignee: Epigenomics AG, Berlin (DE)

(21) Appl. No.: 11/996,267

(22) PCT Filed: Jul. 10, 2006
(86) PCT No.: PCT/EP06/07067

§ 371 Date: Jun. 16, 2008

(30) Foreign Application Priority Data
Jul. 18, 2005 (EP) PCT/EP2005/007830
Sep. 29, 2005 (EP) 05021331.3
Oct. 17, 2005 (EP) 05090289.9
Dec. 23, 2005 (EP) 05090346.7
Jun. 15, 2006 (EP) 06090110.5

(51) Int. Cl. C40B 50/06; G01N 33/53; C12Q 1/68

(52) U.S. Cl. 506/26; 435/7.1; 435/6

(57) ABSTRACT

The present invention relates to compositions and methods for cancer diagnostics, including but not limited to, so-called “pan cancer markers”. In particular, the present invention provides methods of identifying methylation patterns in genes associated with specific cancers, and their related uses. In another aspect, the present invention provides methods of selecting and combining useful sets of pan cancer markers.

### FIELD OF THE INVENTION

The present invention relates to compositions and methods for cancer diagnostics. In particular, the present invention provides methods of identifying methylation patterns in genes associated with specific cell proliferative disorders, including but not limited to cancers, and their related uses. In another aspect, the present invention provides methods of selecting and combining useful sets of markers.

### SEQUENCE LISTING

A Sequence Listing has been provided on compact disc (1 of 1) as a file, entitled seq-prot.txt and which is incorporated by reference herein in its entirety. For the purposes of the present invention, all references as cited herein are incorporated by reference in their entireties.

### BACKGROUND

Several diagnostic tests are used to rule out, confirm, characterize and/or monitor cancer. For many cancers, the most definitive way to do this is to take a small sample of the suspect tissue and look at it under a microscope i.e. a biopsy. However, many biopsies are invasive, unpleasant procedures with their own associated risks, such as pain, bleeding, infection, and tissue or organ damage. In addition, if a biopsy does not result in an accurate or large enough sample, a false negative or misdiagnosis can result, often requiring that the biopsy be repeated. Accordingly there exists a need in the art for improved methods to detect, characterize, and monitor specific types of cancer.

In order to do so, an important goal for many scientists involved in oncology research is the identification of specific and sensitive tumor markers. Commonly used markers for immunohistochemistry in tissues are e.g. cytokeratins (e.g., K19, K20). For high-throughput screening, circulating protein markers that are secreted or shed from the surface of tumor cells are particularly preferred. Carcinoembryonic antigen in colorectal cancer, CA 15-3 and HER-2/neu oncoprotein in breast cancer, PSA in prostate cancer and CA 125 in ovarian cancer all give an indication of the presence of a tumor and enable the detection of tumor cells, furthermore they are used to monitor therapy or recurrence of disease. Histological and immunohistochemical approaches are routinely implemented to identify nodal metastases for staging purposes.

The high rate of disease recurrence in node-negative patients raises the question if current protocols provide sufficient sensitivity and if other tissues (bone marrow, blood) should be examined to discover occult micrometastases. Molecular strategies for the detection of nucleic acid markers are of high interest due to their high sensitivity.

PCR-based techniques specifically amplify DNA sequences and provide a highly sensitive diagnostic platform minimizing the amount of starting material needed. Several genetic alterations acquired by neoplastic cells can be used for their identification. Cancer-specific transcribed gene products have been used to detect the presence of a low concentration of tumor cells.

Nucleic acid-based assays are currently being developed for detecting the presence or absence of known tumor marker proteins in blood or other bodily fluids, or of mRNAs of known tumor related genes. Such assays are distinguished from those based on screening DNA for mutations indicative of hereditary diseases, wherein not only mRNA but also genomic DNA can be analyzed, but wherein no information can be gathered on the actual condition of the patient.

For detection of acute disease status using marker gene approaches, the analyzed DNA must be derived from a diseased cell, such as a tumor cell. The detection of cancer specific alterations of genes involved in carcinogenesis (e.g., oncogene mutations or deletions, tumor suppressor gene mutations or deletions, or microsatellite alterations) facilitates determining the probability that a patient carries a tumor or not (e.g., WO 95/16792 or U.S. Pat. No. 5,952,170 to Stroun et al.). Kits, in some instances, have been developed that allow for efficient and accurate screening of multiple samples. Such kits are not only of interest for improved preventive medicine and early cancer detection, but also utility in monitoring a tumors progression/regression after therapy.

In contrast to DNA detection, however, RNA detection requires special treatment of clinical specimens to protect RNA material from degradation and reverse transcription prior to PCR amplification. Despite very promising studies, the success of PCR-based tests still seems to be hampered by the lack of specific markers with sufficient coverage in the tumor population and the required tissue processing protocols, which are often not compatible with established pathological assays.

In the past few years the detection of minimal residual disease in bone marrow has been shown to be able to provide a valuable new prognostic tool. Standardizations of protocols and procedures are needed in order to compare different studies and to evaluate new diagnostic approaches. Statistically significant data still has to be generated in order to answer the question whether detection of circulating tumor cells in the blood can predict relapse and survival. Technical considerations about blood processing and chosen tumor markers are needed to achieve necessary sensitivity and specificity for clinically relevant studies.

Technical advances have to be pursued in different tissue types to increase detection sensitivity. The establishment of specific detection strategies that use and find the appropriate markers is required for different tumor types, but also for different cancer subsets. Breast cancer is a good example of the heterogeneity of malignant diseases and demonstrates the inability of a single marker to detect all malignancies. The application of several, complementing markers might be necessary to successfully establish acceptable detection sensitivity throughout tumor populations. The design and implementation of multimarker assays requires careful technical considerations including innovative detection strategies (e.g., multicolor approaches) and particular emphasis on consistent specificity. The clinical application of new technologies that promise high sensitivity for the detection of circulating cancer cells still has to be conclusively demonstrated. Therefore, a standardization of protocols is required and most importantly highly specific tumor markers that detect heterogeneous tumor populations are needed.

Microarray-based expression profiling has emerged as a very powerful approach for broad evaluation of gene expression in various systems. However, this approach has its limitations, and one of the most important is the requirement of a certain minimal amount of mRNA: if it is below a certain level due to low promoter activity, short half-life of mRNA, or small amounts of starting material expression of the gene cannot be unambiguously detected. An additional concern is the stability of RNA, which in many cases is difficult to control (e.g., for surgically removed tissue samples), so that the absence of a signal for a certain gene might reflect artificially introduced degradation rather than genuine decrease in expression.

The genome contains approximately 40 million methylated cytosine (5-methylcytosine) bases, otherwise referred to herein as “fifth” bases, which are followed immediately by a guanine residue in the DNA sequence, with CpG dinucleotides comprising about 1.4% of the entire genome. An unusually high proportion of these bases is located in the regulatory and coding regions of genes. Methylation of cytosine residues in DNA is currently thought to play a direct role in controlling normal cellular development. Various studies have demonstrated that a close correlation exists between methylation and transcriptional inactivation. Regions of DNA that are actively engaged in transcription, however, lack 5-methylcytosine residues.

DNA is a much more stable milieu for analysis, and DNA methylation in regions with increased density of CpG dinucleotides (CpG islands) has been shown to correlate inversely with corresponding gene expression when such CpG islands are located in the promoter and/or the first exon of the gene. A number of techniques have been developed for methylation analysis; arguably the most popular of them-methylation-specific PCR or MSP-takes advantage of modification of unmethylated cytosines by bisulfite and alkali which results in their conversion to uracils, changing their partners from guanine to thymine. This change can be detected by PCR with primers that contain appropriate substitutions. A substantial amount of data on gene-specific methylation has been acquired using MSP.

Several markers have been described in the state of the art which are characteristic for the occurrence of cancer. GSTP1, for example, was described as a methylation related marker for prostate cancer, RASSF1A was described as a methylation related marker for breast cancer, APC was described as a marker for lung cancer (Usadel et al Cancer Research 6:371-375, 2002) etc. Nevertheless, these markers are not specific for the type of cancer for which they have been initially described. Indeed, GSTP1 is also methylated in liver cancer, and RASSF1A also in lung cancer and APC also in colon cancer (Hiltunen et al.). Thus, an analysis of body fluid samples would not provide a diagnosis that could determine which organ is afflicted with cancer.

Methylation patterns, comprising multiple CpG dinucleotides, also correlate with gene expression, as well as with the phenotype of many of the most important common and complex human diseases. Methylation positions have, for example, not only been identified that correlate with cancer, as has been corroborated by many publications, but also with diabetes type II, arteriosclerosis, rheumatoid arthritis, and disease of the CNS. Likewise, methylation at other positions correlates with age, gender, nutrition, drug use, and probably a whole range of other environmental influences. Methylation is the only flexible (reversible) genomic parameter under exogenous influence that can change genome function, and hence constitutes the main (and so far missing) link between the genetics of disease and the environmental components that are widely acknowledged to play a decisive role in the etiology of virtually all human pathologies that are the focus of current biomedical research.

Methylation plays a n important role in disease analysis because methylation positions vary as a function of a variety of different fundamental cellular processes. Additionally, however, many positions are methylated in a stochastic way, that does not contribute any relevant information.

Methylation content, levels, profiles and patterns. Genomic methylation can be characterized in distinguishable terms of methylation content, methylation level and methylation patterns. “Methylation content,” or “5-methylcytosine content,” as used herein refers to the total amount of 5-methylcytosine present in a DNA sample (i.e., a measure of base composition), and provides no information as to distribution of the fifth bases. Methylation content of the genome has been shown to differ, depending on the tissue source of the analyzed DNA (Ehrlich M, et al., Nucleic Acids Res. 10: 2709, 1982). However, while Ehrlich et al. showed tissue- and cell specific differences in methylation content among seven different normal human tissues and eight different types of homogeneous human cell populations, their analysis was neither specific with respect to particular genome regions, nor with respect to particular CpG positions. No genes or CpG positions were selected for the analysis, or identified by the analysis that could serve as markers for tissue or cell identification. Rather, only the level of the overall degree of genomic methylation (methylation content) was determined.

“Methylation level” or “methylation degree,” by contrast, refers to the average amount of methylation present at an individual CpG dinucleotide. Measurement of methylation levels at a plurality of different CpG dinucleotide positions creates either a methylation profile or a methylation pattern.

A methylation profile is created when average methylation levels of multiple CpGs (scattered throughout the genome) are collected. Each single CpG position is analyzed independently of the other CpGs in the genome, but is analyzed collectively across all homologous DNA molecules in a pool of differentially methylated DNA molecules (Huang et al., in The Epigenome, S. Beck and A. Olek, eds., Wiley-VCH Weinheim, p 58, 2003).

A methylation pattern, by contrast, is composed of the individual methylation levels of a number of CpG positions in proximity to each other. For example, a full methylation of 5-10 closely linked CpG positions may comprise a methylation pattern that, while rare, may be specific for a specific DNA source.

Prior art correlations involving DNA methylation. A correlation of individual gene methylation patterns with specific tissues has been suggested in the art (Grunau et al., Hut7l Mol. Gen. 9: 2651-2663, 2000). However, in this study, methylation patterns of only four specific genes were analyzed in tissues from only two different individuals, and the aim of the study was to analyze the correlation between known gene expression levels and their respective methylation patterns.

Adorjan et al. published data indicating that tissues such as prostate and kidney could be distinguished by means of methylation markers (Adorjan et al., Nuc. Acids Res. 30: e 21, 2002). This study identified tumor markers, based on analysis of a large number of individuals (relatively large number of samples). Several CpG positions were identified that could be utilized as markers in an appropriate methylation assay to differentiate between kidney and prostate tissue, regardless of the tissue status as being diseased or healthy. However both the Grunau et al., and Adorjan et al. studies offer only a very limited selection of markers to detect a very small proportion of the many known different cell types.

Likewise, patent application WO 03/025215 to Carroll et al., for example, provides a method for creating a map of the methylome (referred to as “a genomic methylation signature”), based on methylation profile analyses, and employing methylation-sensitive restriction enzyme digests and digest-dependant amplification steps. The method description alleges to combine methylation profiling with mapping. This attempt is, however, severely limited for at least three reasons. First, the prior art method provides only a ‘yes or no’ qualitative assessment of the methylation status (methylated or unmethylated) of a cytosine at a genomic CpG position in the genome of interest. Second, the method of Carroll et al. is labor intensive, not being adaptable for high throughput, because it requires a second labor intensive step; namely, after completing the process of restriction enzyme-based methylation analysis to identify a particular amplificate as a potential methylation marker, each of these amplified digestion dependent markers (amplificates) needs to be cloned and sequenced for mapping to the genome.

Third, there are no means described by Carroll et al. for utilizing the generated information in a tissue specific manner. Specifically, while Carroll et al. disclose that specific different tissues of mice have different “methylomes” (WO 03/025215, FIG. 6), and that two different human tissues, sperm cells and blood cells, could be correlated with differing amplification profiles (Id, FIGS. 4 and 10, where CpG positions were identified that were unmethylated in one scenario and methylated in the other), there is no means or enablement to support use of this information as a specific tissue marker.

Protein expression-based prior art approaches. Immunohistochemical assays are utilized as standard methods to determine a cell type or a tissue type of cellular origin in the context of an intact organism. Such methods are based on the detection of specific proteins. For example, the German Center for collection of microorganisms and cell cultures (DSMZ) routinely tests the expression of tissue markers on all arriving human cell lines with a panel of well-characterized monoclonal antibodies (mAbs) (Quentmeier H, et al., J Histochem. Cytochem. 49: 1369-1378, 2001). Generally, the expression pattern of histological markers reflects that of the originating cell type. However, expression of the proteins, carbohydrate or lipid structures that are detected by individual mAbs, is not always stable over a long period of time.

Likewise, immunophenotyping, which can be performed both to confirm the histological origin of a cell line, and to provide customers with useful information for scientific applications, is based on testing the stability and intensity of cell surface marker expression. Immunophenotyping typically includes a two-step staining procedure, wherein antigen-specific murine mAbs are added to the cells in the first step, followed by assessment of binding of the mAbs by an immunofluorescence technique using FITC-conjugated anti-mouse Ig secondary antisera. Distribution of antigens is analyzed by flow-cytometry and/or light microscopy.

Therefore the process of determining a cell type or tissue type using these expression-based methods is not trivial, but rather complex. The more marker proteins are known the more precisely a cell's status of origin can be determined. Without the use of molecular biology techniques, such as RNA-based cDNA/oligo-microarrays or a complex proteomics experiment, which enable the simultaneous view of a higher number of changes, the identification of a specific cell type would require a sequence of tedious and time-consuming assays to detect a rather complex protein expression pattern. Finally, proteomic approaches have not overcome basic difficulties, such as reaching sufficient sensitivity.

RNA expression-based prior art approaches. RNA-based techniques to analyze expression patterns are well-known and widely used. In particular, microarray-based expression analysis studies to differentiate cell types and organs have been described, and used to show that precise patterns of differentially expressed genes are specific for a particular cell type.

A system of cluster analysis for genome-wide expression data from DNA microarray hybridization is described by Eisen et al. Proc. Natl. Acad. Sci. USA. 95: 14863-8, 1998. Eisen et al. teach clustering of gene expression data groups together, especially data for genes of known similar function, and interpretation of the patterns found as an indicator of the status of cellular processes. However, the teachings of Eisen are in the context of yeast and, therefore, cannot be extended to identify tissue or organ markers useful in human beings or other more developmentally complex organisms and animals. Likewise such teachings cannot be extended into the area of human disease prognostics and diagnostics. Similarly, Ben-Dor et al. describe an expression-based approach for tissue classification in humans. However, as in nearly all related publications, the scope is limited to markers for the identification of tumors (Ben-Dor et al. J Comput Biol. 7: 559-83, 2000).

Likewise, Enard et al. recently published a comparative analysis of expression patterns within specific tissue samples across different species, teaching different mRNA and protein expression patterns between different individuals of one species (intra-specific variation), as well as between different species (inter-specific variation). Enard et al. did not however, teach or enable use of such expression levels for distinguishing between or among different tissues.

Lack of acceptance of prior art methods by regulatory agencies. Significantly, regulatory agencies are currently not willing to accept a technology platform relying on an expression microarray due to the above-described shortcomings.

U.S. Pat. No. 6,581,011 to Tissue Informatics Inc., teaches a tissue information database for profiling and classifying a broad range of normal tissues, and illustrates the need in the art for tools allowing classification of a tissue.

Hypermethylation of certain ‘tumor marker’ genes, especially of certain promoter regions thereof, is recognized as an important indicator of the presence or absence of a tumor. Significantly, however, such prior art methylation analyses are limited to those based on determination of the methylation status of known marker genes, and do not extent to genomic regions that have not been previously implicated based on function; ‘tumor marker’ genes are those genes known to play a role in the regulation of carcinogenesis, or are believed to determine the switching on and off of tumorigenesis.

Knowledge of the correlation of methylation of tumor marker genes and cancer is most advanced in the case of prostate cancer. For example, a method using DNA from a bodily fluid, and comprising the methylation analysis of the tumor marker gene GSTP1 as an predictive indicator of prostate cancer has been patented (U.S. Pat. No. 5,552,277).

Significantly, prior art tumor marker screening approaches are limited to certain types of diseases (e.g., cancer types). This is because they are limited to analysis of marker genes, or gene products which are highly specific for a kind of disease, mostly being cancer, when found in a specific kind of bodily fluid. For example, Usadel et al. teach detection of a tumor specific methylation in the promoter region of the adenomatous polyposis coli (APC) gene in serum samples of lung cancer patients, but that no methylated APC promoter DNA is detected in serum samples of healthy donors (Usadel et al. Cancer Research 6: 371-375, 2002). This marker thus qualifies as a reasonable indicator for lung cancer, and has utility for the screening of people diagnosed with lung cancer, or for monitoring of patients after surgical removal of a tumor for developing metastases in their lung.

WO 2005/019477, for example, further describes this particular problem: “Moreover the teachings of Usadel et al. are also limited by the fact that the epigenetic APC gene alterations are not specific for lung cancer, but are common in other cancer, for example, ingastrointestinal tumor development. Therefore, a blood screen with only APC as a tumor marker has limited diagnostic utility to indicate that the patient is developing a tumor, but not where that tumor would be located or derived from. Consequently, a physician would not be informed with respect to a more detailed diagnosis of an specific organ, or even with respect to treatment options of the respective medical condition; most of the available diagnostic or therapeutic measures will be organ- or tumor source-specific. This is particularly true where the lesion is small in size, and it will be extremely difficult to target further diagnostics and therapies. Given the nature of marker genes as previously implicated genes, prior art use of marker genes for early diagnosis has occurred where a specific medical condition is already in mind. For example, a physician suspicious of having a patient who developed a colon cancer, can have the patient's stool sample tested for the status of a cancer marker gene like K-ras. A patient suspected as having developed a prostate cancer, may have his ejaculate sample tested for a prostate cancer marker like GSTPi.”

Significantly, however, there is no prior art method described for efficient and effective generally screening of patients, or bodily fluids thereof where the patient has no specific prior indication or suspicion as to which organ or tissue might have developed a cell proliferative disease (e.g., an individual previously exposed to a high level of radiation).

Thus, there is a substantial need in the art including from the clinical perspective, to identify cell or tissue type and/or cell or tissue source. For example, there is a need in the art for efficient and effective typing of disseminated tumor cells, for determining the tissue of origin (i.e., the type of tissue or organ the tumor was derived from). No such tools or methods, apart from a few disclosed isolated markers, are available in the prior art. Likewise, no generally applicable prior art methods are available for determining the cell- or tissue-type from which a genomic DNA sample was derived. In addition, the nature of the disease of the organ remains open. In case of colon-specific markers, also an inflammation of the colon could be present, in this case a subsequent diagnosis for the determination of the particular disease of the organ has to follow.

### SUMMARY OF THE INVENTION

In one aspect thereof, the object according to the present invention is solved by a method for diagnosing a proliferative disease in a subject comprising: a) providing a biological sample from a subject, b) detecting the presence, absence, abundance and/or expression of one or more markers and determining therefrom upon the presence or absence of a proliferative disease; and c) detecting the presence, absence, abundance and/or expression of one or more cell- or tissue-markers and determining therefrom if said one or more cell- and/or tissue-markers are atypically present, absent or present at above normal levels within said sample; and d) determining the presence or absence of a cell proliferative disorder and location thereof based on the presence, absence, abundance and/or expression as detected in step b) and c). Preferred is a method according to the present invention, further comprising detecting the presence, absence, abundance and/or expression of one or more markers and determining therefrom characteristics of said cell proliferative disorder. Preferred is a method according to the present invention, wherein said proliferative disease is cancer, and in particular selected from soft tissue, skin, leukemia, renal, prostate, brain, bone, blood, lymphoid, stomach, head and neck, colon or breast cancer. Further preferred is a method according to the present invention, wherein said marker is indicative of more than one proliferative disease. Most preferred is a method according to the present invention, wherein said proliferative disease is cancer.

According to the invention, said detecting the expression of one or more marker that is specific for more than one proliferative disease comprises detecting the presence, absence, abundance and/or expression of physiological, genetic and/or cellular expression and/or cell count, preferably said detecting the expression comprises detecting the expression of protein, mRNA expression and/or the presence or absence of DNA methylation in one or more of said markers. Particularly, said detecting the expression of protein comprises marker-specific antibodies, ELISA, cell sorting techniques, Western blot, or the detection of labeled protein, and said measuring the mRNA expression comprises detection of labeled mRNA or Northern blot.

In another aspect thereof, the object according to the present invention is solved by a method for diagnosing a proliferative disease in a subject comprising the steps of: a) providing a biological sample from a subject, said biological sample comprising genomic DNA; b) detecting the level of DNA methylation in one or more markers and determining therefrom upon the presence or absence of a proliferative disease; and c) detecting the level of methylation of one or more markers and determining therefrom if said one or more cell- and/or tissue-markers are atypically present, absent or present at above normal levels within said sample; and d) determining the presence or absence of a cell proliferative disorder and location thereof, based on the level of DNA methylation as detected in step b) and c). Preferably, step b) further comprises comparing said methylation profile to one or more standard methylation profiles, wherein said standard methylation profiles are selected from the group consisting of methylation profiles of non cell proliferative disorder samples and methylation profiles of cell proliferative disorder samples. More preferably, said detecting the presence or absence of DNA methylation comprises the digestion of said genomic DNA with a methylation-sensitive restriction enzyme, followed by multiplexed amplification of gene-specific DNA fragments with CpG islands.

According to the present invention, preferred is a method, wherein the markers of step b) are selected from the group consisting of nucleic acid sequences according to any of SEQ ID NO: 100 to SEQ ID NO: 161. According to the present invention, preferred is a method, wherein the markers of step c) are selected from the group consisting of nucleic acid sequences according to any of SEQ ID NO: 1 to SEQ ID NO: 99 and SEQ ID NO: 844 to SEQ ID NO: 1255.

According to the present invention, preferred is a method according to the present invention, wherein said proliferative disease is selected from psoriasis or cancer, and in particular selected from soft tissue, skin, leukemia, renal, prostate, brain, bone, blood, lymphoid, stomach, head and neck, colon or breast cancer.

In another preferred aspect thereof, the object according to the present invention is solved by a method, wherein said characterizing of said cancer comprises detecting the presence or absence of chemotherapy resistant cancer.

In yet another preferred aspect thereof, the object according to the present invention is solved by a method, wherein said chemotherapy is a non-steroidal selective estrogen receptor modulator.

In yet another aspect preferred thereof, the object according to the present invention is solved by a method, wherein said characterizing cancer comprises determining a chance of disease-free survival, and/or monitoring disease progression in said subject.

In yet another preferred aspect thereof, the object according to the present invention is solved by a method, wherein said characterizing cancer comprises determining metastatic disease by identifying tissue markers in said sample that are foreign to the tissue from which said sample is taken from.

In yet another preferred aspect thereof, the object according to the present invention is solved by a method, wherein said characterizing cancer comprises determining relapse of the disease after complete resection of the tumor in said subject by identifying tissue markers and cancer markers in said sample that are identical to the removed tumor.

Further preferred is a method according to the present invention, wherein said biological sample is a biopsy sample or a blood sample. Even further preferred is a method according to the present invention, wherein said proliferative disease is in the early pre-clinical stage exhibiting no clinical symptoms.

Still further preferred is a method according to the present invention, wherein said detecting the presence or absence of DNA methylation comprises the digestion of said genomic DNA with a methylation-sensitive restriction enzyme followed by multiplexed amplification of gene-specific DNA fragments with CpG islands. Still further preferred is a method according to the present invention, wherein said detecting the presence or absence of DNA methylation comprises treatment of said genomic DNA with one or more reagents suitable to convert 5-position unmethylated cytosine bases to uracil or to another base that is detectably dissimilar to cytosine in terms of hybridization properties. Still further preferred is such a method according to the present invention, wherein said markers of step b) are selected from the group consisting of nucleic acid sequences according to any of SEQ ID NO: 100 to SEQ ID NO: 161, and SEQ ID NO: 360 to SEQ ID NO: 483, and SEQ ID NO: 682 to SEQ ID NO: 805. Still further preferred is such a method according to the present invention, wherein said markers of step c) are selected from the group consisting of the genomic nucleic acid sequences according to any of SEQ ID NO: 1 to SEQ ID NO: 99 or SEQ ID NO: 844 to SEQ ID NO: 1255, or their bisulfite converted variants according to SEQ ID NO: 162 to SEQ ID NO: 359, SEQ ID NO: 484 to SEQ ID NO: 681 and SEQ ID NO: 1256 to SEQ ID NO: 2903.

In yet another preferred aspect thereof, the object according to the present invention is solved by a method for generating a pan-cancer marker panel for the improved diagnosis and/or monitoring of a proliferative disease in a subject, comprising a) providing a biological sample from said subject suspected of or previously being diagnosed as having a proliferative disease, b) providing a first set of one or more markers indicative for proliferative disease, c) determining the presence, absence, abundance and/or expression of said one or more markers of step b); d) providing a first set of tissue markers, e) determining the expression of said one or more markers of step d), and f) generating a pan-cancer marker panel that is specific for said proliferative disease in said subject by selecting those markers that are differently expressed in said subject when compared to an expression profile of a healthy sample.

According to the invention, said detecting the presence, absence, abundance and/or expression of one or more marker that is specific for more than one proliferative disease comprises detecting the expression of physiological, genetic and/or cellular expression and/or cell count, preferably said detecting the expression comprises detecting the expression of protein, mRNA expression and/or the presence or absence of DNA methylation in one or more of said markers. Particularly, said detecting the expression of protein comprises marker-specific antibodies, ELISA, cell sorting techniques, Western blot, or the detection of labeled protein, and said measuring the mRNA expression comprises detection of labeled mRNA or Northern blot.

According to the present invention, preferred is a method, wherein said marker is indicative of more than one proliferative disease. According to the present invention, preferred is a method, wherein said markers of step b) are selected from the group consisting of nucleic acid sequences according to any of SEQ ID NO: 100 to SEQ ID NO: 161. According to the present invention, preferred is a method, wherein the markers of step c) are selected from the group consisting SEQ ID NO: 1 to SEQ ID NO: 99 and SEQ ID NO: 844 to SEQ ID NO: 1255.

According to the present invention, preferred is a method, wherein said proliferative disease is selected from psoriasis or cancer, in particular from soft tissue, skin, leukemia, renal, prostate, brain, bone, blood, lymphoid, stomach, head and neck, colon or breast cancer.

More preferred is a method according to the present invention, wherein the biological sample to be analyzed is a biopsy sample or a blood sample. Also preferred is a method according to the present invention, wherein said DNA methylation comprises CpG methylation and/or imprinting.

Most preferred is a method according to the present invention, wherein said proliferative disease is in the early pre-clinical stage exhibiting no clinical symptoms.

In yet another preferred aspect thereof, the object according to the present invention is solved by a method according to the present invention, wherein said detecting the presence or absence of DNA methylation comprises the digestion of said genomic DNA with a methylation-sensitive restriction enzyme, followed by multiplexed amplification of gene-specific DNA fragments with CpG islands.

In yet another preferred aspect thereof, the object according to the present invention is solved by an improved method for the treatment of a proliferative disease, comprising a method as describe hereinabove, and selecting a suitable treatment regimen for said proliferative disease to be treated. Again, said proliferative disease can be selected from soft tissue, skin, leukemia, renal, prostate, brain, bone, blood, lymphoid, stomach, head and neck, colon or breast cancer.

In yet another preferred aspect thereof, the object according to the present invention is solved by a kit for diagnosing a proliferative disease in a subject, wherein said kit comprises reagents for detecting the expression of one or more marker indicative for more than one proliferative disease; and reagents for localizing the proliferative disease and/or characterizing the type of proliferative disease by detecting specific tissue markers based on nucleic acid-analysis. Preferably, said kit further comprises instructions for using said kit for characterizing cancer in said subject. More preferably, in said kit said reagents comprise reagents for detecting the presence or absence of DNA methylation. Further preferred is a kit according to the present invention, wherein the markers are selected from the group consisting of nucleic acid sequences according to any of SEQ ID NO: 1 to SEQ ID NO: 161 and SEQ ID NO: 844 to SEQ ID NO: 2903, and chemically pretreated sequences thereof.

### DETAILED DESCRIPTION OF THE INVENTION

### Definitions

To facilitate an understanding of the present invention, a number of terms and phrases are defined below:

The term “epitope” as used herein refers to that portion of an antigen that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject suspected of having cancer” refers to a subject that presents one or more symptoms indicative of a cancer (e.g., a noticeable lump or mass). A subject suspected of having cancer may also have on or more risk factors. A subject suspected of having cancer has generally not been tested for cancer. However, a “subject suspected of having cancer” encompasses an individual who has received an initial diagnosis (e.g., a CT scan showing a mass) but for whom the sub-type or stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, genetic predisposition, environmental expose, pre-existing non cancer diseases, and lifestyle.

As used herein, the term “stage of cancer” refers to a numerical measurement of the level of advancement of a cancer. Criteria used to determine the stage of a cancer include, but are not limited to, the size of the tumour, whether the tumour has spread to other parts of the body and where the cancer has spread (e.g., within the same organ or region of the body or to another organ).

As used herein, the term “sub-type of cancer” refers to different types of cancer that effect the same organ (ductal cancer, lobular cancer, and inflammatory breast cancer are sub-types of breast cancer.

As used herein, the term “providing a prognosis” refers to providing information regarding the impact of the presence of cancer (e.g., as determined by the diagnostic methods of the present invention) on a subject's future health (e.g., expected morbidity or mortality).

As used herein, the term “subject diagnosed with a cancer” refers to a subject having cancerous cells. The cancer may be diagnosed using any suitable method, including but not limited to, the diagnostic methods of the present invention.

As used herein, the term “instructions for using said kit for detecting of a proliferative disease, in particular cancer, in said subject” includes instructions for using the reagents contained in the kit for the detection and characterization of a proliferative disease, in particular cancer, in a sample from a subject. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination. Additional information is available at the Internet web page of the U.S. FDA.

As used herein, the term “detecting the presence or absence of DNA methylation” refers to the detection of DNA methylation in the promoter and/or regulatory regions of one or more genes (e.g., cancer markers of the present invention) of a genomic DNA sample. The detecting may be carried out using any suitable method, including, but not limited to, those disclosed herein.

As used herein, the term “detecting the presence or absence of chemotherapy resistant cancer” refers to detecting a DNA methylation pattern characteristic of a tumor that is likely to be resistant to chemotherapeutic agents (e.g., non-steroidal selective estrogen receptor modulators (SERMs)).

As used herein, the term “determining the chance of disease-free survival” refers to the determining the likelihood of a subject diagnosed with cancer surviving without the recurrence of cancer (e.g., metastatic cancer). In some embodiments, determining the chance of disease free survival comprises determining the DNA methylation pattern of the subject's genomic DNA.

As used herein, the term “determining the risk of developing metastatic disease” refers to likelihood of a subject diagnosed with cancer developing metastatic cancer. In some embodiments, determining the risk of developing metastatic disease comprises determining the DNA methylation pattern of the subject's genomic DNA.

As used herein, the term “monitoring disease progression in said subject” refers to the monitoring of any aspect of disease progression, including, but not limited to, the spread of cancer, the metastasis of cancer, and the development of a pre-cancerous lesion into cancer. In some embodiments, monitoring disease progression comprises determining the DNA methylation pattern of the subject's genomic DNA.

As used herein, the term “methylation profile” refers to a presentation of methylation status of one or more marker genes in a subject's genomic DNA. In some embodiments, the methylation profile is compared to a standard methylation profile comprising a methylation profile from a known type of sample (e.g., cancerous or non-cancerous samples or samples from different stages of cancer). In some embodiments, specific methylation profiles are generated using the methods of the present invention. The profile may be presented as a graphical representation (e.g., on paper or on a computer screen), a physical representation (e.g., a gel or array) or a digital representation stored in computer memory.

As used herein, the term “nucleic acid molecule” refers to any nucleic acid containing molecule including, but not limited to DNA or RNA. The term encompasses sequences that include any of the known base analogs of DNA and RNA including, but not limited to, 4-acetylcytosine, 8-hydroxy-N-6-methyladenosine, aziridinyl cytosine, pseudo isocytosine, 5-(carboxyhydroxylmethyl) uracil, 5-fluorouracil, 5-bromouracil, 5-carboxymethyl aminomethyl-2-thiouracil, 5-carboxymethyl aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonyl methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbour in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element or the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc. (defined infra).

Transcriptional control signals in eukaryotes comprise “promoter” and “enhancer” elements. Promoters and enhancers consist of short arrays of DNA sequences that interact specifically with cellular proteins involved in transcription (T. Maniatis et al., Science 236:1237 [1987]). Promoter and enhancer elements have been isolated from a variety of eukaryotic sources including genes in yeast, insect and mammalian cells, and viruses (analogous control elements, i.e., promoters, are also found in prokaryote). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and T. Maniatis et al., supra). For example, the SV40 early gene enhancer is very active in a wide variety of cell types from many mammalian species and has been widely used for the expression of proteins in mammalian cells (Dijkema et al., EMBO J. 4:761 [1985]). Two other examples of promoter/enhancer elements active in a broad range of mammalian cell types are those from the human elongation factor 1[alpha] gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl, Acad. Sci. USA 79:6777 [1982]) and the human cytomegalovirus (Boshart et al., Cell 41:521 [1985]). Some promoter elements serve to direct gene expression in a tissue-specific manner.

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques such as cloning and recombination) such that transcription of that gene is directed by the linked enhancer/promoter.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described below.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tm.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5* SSPE (43.8 g/l NaCl, 6.9 g/l NaH2PO4 H2O and 1.85 g/l EDTA, pH adjusted to 7.4 with NaOH), 0.5% SDS, 5* Denhardt's reagent and 100 μg/ml denatured salmon sperm DNA followed by washing in a solution comprising 0.1* SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5* SSPE (43.8 g/l NaCl, 6.9 g/l NaH2PO4 H2O and 1.85 g/l EDTA, pH adjusted to 7.4 with NaOH), 0.5% SDS, 5* Denhardt's reagent and 100 μg/ml denatured salmon sperm DNA followed by washing in a solution comprising 1.0* SSPE, 1.0% SDS at 42° C. when a probe of about 500 nucleotides in length is employed.

“Low stringency conditions” comprise conditions equivalent to binding or hybridization at 42° C. in a solution consisting of 5* SSPE (43.8 g/l NaCl, 6.9 g/l NaH2PO4 H2O and 1.85 g/l EDTA, pH adjusted to 7.4 with NaOH), 0.1% SDS, 5* Denhardt's reagent [50* Denhardt's contains per 500 ml: 5 g Ficoll (Type 400, Pharamcia), 5 g BSA (Fraction V; Sigma)] and 100 [mu]g/ml denatured salmon sperm DNA followed by washing in a solution comprising 5* SSPE, 0.1% SDS at 42° C. when a probe of about 500 nucleotides in length is employed.

It is well known in the art that numerous equivalent conditions may be employed to provide low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, conditions that promote hybridization under conditions of high stringency (e.g., increasing the temperature of the hybridization and/or wash steps, the use of formamide in the hybridization solution, etc.) are known in the art (see definition above for “stringency”).

“Amplification” is a specific case of nucleic acid replication characterised by template specificity. Template specificity (affinity for a nucleic acid template) is independent of fidelity of replication (i.e., synthesis of a polynucleotide sequence) and nucleotide (ribo- or deoxyribo-) specificity. Template specificity is frequently described in terms of “target” specificity. Target sequences are sequences that are preferentially amplified, and many amplification techniques are specifically adapted to ensure preferential and specific amplification of said sequences.

Template specificity is achieved in most amplification techniques by the choice of amplification enzyme. Preferred are amplification enzymes that under suitable conditions will only amplify specific nucleic acid sequences in a heterogeneous mixture of nucleic acids. For example, in the case of Qβ replicase, MDV-1 RNA is the specific template for the replicase (Kacian et al., Proc. Natl. Acad. Sci. USA 69:3038 [1972]). Other nucleic acids will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighbouring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The terms “overexpression” and “overexpressing” and grammatical equivalents, if used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

The term “tissue” in this context is meant to describe a group or layer of cells that are structurally and/or functionally similar and that work together to perform a specific function.

The term “oligomer” encompasses oligonucleotides, PNA-oligomers and DNA oligomers, and is used whenever a term is needed to describe the alternative use of an oligonucleotide or a PNA-oligomer or DNA-oligomer, which cannot be described as oligonucleotide. Said oligomer can be modified as it is commonly known and described in the art. The term “oligomer” also encompasses oligomers carrying at least one detectable label, and preferably fluorescence labels are understood to be encompassed. It is however also understood that the label can be of any kind that is known and described in the art.

The term “Observed/Expected Ratio” (“O/E Ratio”) refers to the frequency of CpG dinucleotides within a particular DNA sequence, and corresponds to the [number of CpG sites/(number of C bases×number of G bases)]×band length for each fragment.

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5. CpG islands are typically, but not always, between about 0.2 to about 1 kb in length, and may be as large as about 3 kb in length.

The term “methylation state” or “methylation status” or “methylation level” refers to the presence or absence of 5-methylcytosine (“5-mCyt”) at one or a plurality of CpG dinucleotides within a DNA sequence.

Methylation states or methylation levels at one or more CpG methylation sites within a single allele's DNA sequence include “unmethylated,” “fully-methylated” and “hemi-methylated.” The term “hemi-methylation” or “hemimethylation” refers to the methylation state of a CpG methylation site, where only one strand's cytosine of the CpG dinucleotide sequence is methylated. The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both “DNA microarrays” and “DNA chip (s),” and encompasses all art-recognized solid supports, and all art-recognized methods for affixing nucleic acid molecules thereto or for synthesis of nucleic acids thereon.

“Genetic parameters” as used herein are mutations and polymorphisms of genes and sequences further required for gene regulation. Exemplary mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlate with the DNA methylation.

The term “bisulfite reagent” refers to a reagent comprising bisulfite, sulfite, hydrogen sulfite or combinations thereof, useful as disclosed herein to distinguish between methylated and unmethylated CpG dinucleotide sequences.

The term “Methylation assay” refers to any assay for determining the methylation state or methylation level of one or more CpG dinucleotide sequences within a sequence of DNA.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57: 594-599, 1997.

The term “MethyLight” refers to the art-recognized fluorescence-based real-time PCR technique described by Eads et al., Cancer Res. 59: 2302-2306, 1999.

The term “HeavyMethyl” assay, in the embodiment thereof implemented herein, refers to a HeavyMethyl/MethyLight assay, which is a variation of the MethyLight assay, wherein the MethyLight assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25: 2529-2531, 1997.

The term “MSP” (Methylation-specific PCR) refers to the art-recognized methylation assay described by Herman et al. Proc. Natl. Acad. Sci. USA 93: 9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25: 2532-2534, 1997.

The term “MCA” (Methylated CpG Island Amplification) refers to the methylation assay described by Toyota et al., Cancer Res. 59: 2307-12, 1999, and in WO 00/26401A1.

With respect to the dinucleotide designations within the phrase “CpG, tpG and Cpa” a small “t” is used to indicate a thymine at a cytosine position, whenever the cytosine was transformed to uracil by pretreatment, whereas, a capital “T” is used to indicate a thymine position that was a thymine prior to pretreatment). Likewise, a small “a” is used to indicate the adenine corresponding to such a small “t” located at a cytosine position, whereas a capital “A” is used to indicate an adenine that was adenine prior to pretreatment.

In the context of the present invention, the term “marker” refers to a distinguishing of a characteristic that may be detectable if present in blood, serum or other bodily fluids, or preferably in cell and/or tissues that is reflective of the presence of a particular condition (in particular a disease). The characteristic may be a phenotypical characteristic, such as cell count, cell shape, viability, presence/absence of circulating tumor cells and/or a physiological characteristic, such as a protein, an enzyme, an RNA molecule or a DNA molecule. The term may alternately refer to a specific characteristic of said substance, such as, but not limited to, a specific methylation pattern, making the characteristic distinguishable from otherwise identical characteristics. Examples for markers are “pan-cancer markers” and “cell- or tissue-markers”, as described below. Preferred markers can be identified from tables 1 and 2, herein below.

The term “pan-cancer marker” refers to a distinguishing or characteristic substance (such as a marker) that may be detectable if present in blood, serum or other bodily fluids, or preferably in tissues that is reflective of the presence of proliferative disease. Pan-cancer markers are characterized by the fact that they reflect the possibility of the presence of more than one proliferative diseases in organs or tissues of the patient and/or subject. Thus, pan-cancer markers are not specific for a single proliferative disease being present in an organ or tissue, but are specific for more than one proliferative disease for said subject. The substance may, for example, be cell count, presence/absence of circulating tumor cells, a protein, an enzyme, an RNA molecule or a DNA molecule that is suitable to used as a marker. The term may alternately refer to a specific characteristic of said substance, such as, but not limited to, a specific methylation pattern, making the substance distinguishable from otherwise identical substances. A high level of a tumor marker may indicate that cancer is developing in the body. Typically, this substance is derived from the tumor itself. Examples of pan-cancer tumor markers include, but are not limited to CEA (ovarian, lung, breast, pancreas, and gastrointestinal tract cancers), and GSTPi (liver and prostate cancer). Further markers can be identified from table 2, herein below.

The term “cell- or tissue-marker” refers to a distinguishing or characteristic substance of a specific cell type or tissue that may be detectable if present in blood or other bodily fluids, but preferably in cells of specific tissues. The substance may for example be a protein, an enzyme, a RNA molecule or a DNA molecule. The term may alternately refer to a specific characteristic of said substance, such as but not limited to a specific methylation pattern, making the substance distinguishable from otherwise identical substances. A high level of a tissue marker found in a cell may mean said cell is a cell of that respective tissue. A high level of a cell- or tissue-marker found in a bodily fluid may mean that a respective type of tissue is either spreading cells that contain said marker into the bodily fluid, or is spreading the marker itself into the blood or other bodily fluids. Further markers can be identified from table 1, herein below.

The term “nucleic acid-analysis” refers to an analysis of the presence and/or expression of a marker that is based, at least in part, on an analysis of nucleic acid molecule(s) that is (are) specific for said marker. One preferred example of nucleic acid-analysis would be methylation analysis of the DNA of the particular marker.

The term “localizing the proliferative disease” refers to an analysis of a marker that may be found in a sample, wherein said marker is known to be expressed in one or more cells of specific tissues. A high level of a tissue marker found in a cell means that this said cell is a cell of that respective tissue. This information (or an information derived from several markers) is used in order to localize the proliferative disease inside the body of the patient as being found in one or several particular tissue(s).

The term “ESME” refers to a novel and particularly preferred software program that considers or accounts for the unequal distribution of bases in bisulfite converted DNA and normalizes the sequence traces (electropherograms) to allow for quantitation of methylation signals within the sequence traces. Additionally, it calculates a bisulfite conversion rate, by comparing signal intensities of thymines at specific positions, based on the information about the corresponding untreated DNA sequence (see U.S. publication number 2004-0023279, and EP 1 369 493 (in German), both incorporated by reference herein in their entirety).

Unless defined otherwise, all technical and scientific terms used herein have the same meaning as commonly understood by one of ordinary skill in the art to which the invention pertains. Although any methods and materials similar or equivalent to those described herein can be used for testing of the present invention, the preferred materials and methods are described herein. All documents cited herein are thereby incorporated by reference.

In one—and the major—aspect thereof, the present invention provides a particular method for diagnosing a proliferative disease in a subject. The method generally comprises the steps of: providing a biological sample from a subject, detecting the presence, absence, abundance and/or expression of one or more markers that indicate proliferative disease in said sample; and localizing the proliferative disease and/or characterizing the type of proliferative disease by detecting specific tissue markers wherein the detection of said tissue markers is based on nucleic acid-analysis.

The particular advantage of the solution according to the present invention is based—first—on the use of markers for the diagnosis that are not specific for one type of proliferative disease (for example, cancer) which sometimes (and also herein) are designated as “pan-cancer markers”. Those markers can, for example, exhibit a change in methylation in nearly all types of cancers (or are, for example, overexpressed), or combinations of those markers can be (specifically and preferably) combined into a pan-cancer panel and used in order to efficiently and sensitively detect any proliferative disease (cancerous disease), or at least many different proliferative diseases (cancerous diseases). This needs not to limited to a methylation analysis, but can also be combined with the analysis of other markers. Second, for a localisation of the cancer/determination of the type of cancer a detection of specific tissue markers based on nucleic acid-analysis is performed, and the two results of the marker analyses are combined in order to provide a localisation of the cancer/determination of the type of cancer (characterisation thereof).

The analysis of the pan-cancer markers has the advantage that they can be very sensitive and specific for a kind of “cancer-yes/no” information, but at the same time need not to give a clear indication about the localisation of the cancer (e.g. need not to be tissue- and/or cell-specific). Thus, this allows for a simplified generation of qualitative and improved diagnostic marker panels for proliferative diseases, since very sensitive and very tissue-specific markers can be combined in such a diagnostic marker panel. Nevertheless, the present method according to the invention, in particular in embodiments for following-up (monitoring) of once identified proliferative diseases, can also include a quantitative analysis of the expression and/or the methylation of a marker or markers as employed (see below).

US 2004/0137474 describes detecting the presence or absence of DNA methylation in DAPK, GSTP, p15, MDR1, Progesterone Receptor, Calcitonin, RIZ, and RARbeta genes, thereby characterizing cancer in a subject to be diagnosed. Furthermore, detecting the presence or absence of DNA methylation in one or more genes selected from the group consisting of S100, SRBC, BRCA, HIN1, Cyclin D2, TMS1, HIC-1, hMLH1E-cadherin, 14-3-3sigma, and MDGI is described.

Regarding the tissue- and/or cell-specific markers, many of such markers are known from the state of the art and are given herein below in Table 2.

Particular preferred are markers for the determination of the tissue(s) that—similarly to preferred pan-cancer markers—rely on an analysis of methylation of particular genes, as described, for example, in WO 2005-019477 “Methods and compositions for differentiating tissues or cell types using epigenetic markers”. Nevertheless, other expression markers can be also used as, for example described in Li-Li Hsiao et al. (A Compendium of Gene Expression in Normal Human Tissues Reveals Tissue-Selective Genes and Distinct Expression Patterns of Housekeeping Genes Physiol. Genomics (Oct. 2, 2001)), Butte et al. (Further defining housekeeping, or “maintenance,” genes Focus on “A compendium of gene expression in normal human tissues” Physiol. Genomics 7: 95-96, 2001), and the HuGE Index: Human Gene Expression Index at http://www.hugeindex.org.

US 2005-048480 describes a method for selecting a gene used as an index of cancer classification, comprising the following steps of: (1) determining expression levels in cancer samples to be tested for at least one of genes each of which expression is altered specifically during cell proliferation, and then comparing the determined expression levels with an expression level of the genes in a control sample, thereby evaluating alterations in expression levels of the genes, wherein the control sample is a normal tissue, or a cancer sample with low malignancy; (2) classifying the cancer samples to be tested into plural numbers of types, based on alterations in expression levels of the genes evaluated in the above step (1) and pathological findings for the cancer samples to be tested; and (3) examining alterations in expressions for plural numbers of genes in each of the cancer samples to be tested classified in the above step (2), to select a gene, wherein expression of said gene is altered independently to genes each of which expression is altered specifically during cell proliferation and expression level of said gene is specifically altered depending on every type of cancer samples to be tested. Preferably, in the step (1), expression levels of genes selected from the group consisting of CDC6 gene and E2F family genes are determined on the basis of levels of mRNAs transcribed from the genes. Nevertheless, US 2005-048480 describes that the expression level shall be used in order to identify the type of cancer, which renders the analysis rather complicated. Tissue identification is not described.

In addition to the advantages as described above, the method according to the present invention can be flexibly used, for example, in several different preferred aspects as follows:
Marker-panels (pan-cancer panels can be combined and provided that in their particular combination of pan-cancer and tissue markers readily and quickly lead to the desired result, e.g. the early pre-clinical diagnosis of certain types of cancer, preferably even before clinical symptoms become evident. Further laborious examinations for the determination of the localisation of the cancer/determination of the type of cancer (characterisation thereof) can be avoided. In addition, an earlier therapy of a cancer usually leads to a higher likelihood of a successful outcome of the therapy.
The method according to the present invention can be used in detecting the presence or absence of chemotherapy-resistant cancer. This method can be performed by monitoring the markers of a pan-cancer panel in order to detect if a particular cancer in a particular tissue is still present or not, or whether the quantitative amount of cancer marker versus tissue marker is changing over the time of an anti-cancer treatment. A quantification can be achieved by, e.g. measuring signal intensity in an ELISA or employing real-time methylation analysis, such as, for example, MethyLight®. In yet another preferred aspect thereof, said chemotherapy is a nonsteroidal selective estrogen receptor modulator.
The method according to the present invention can be used in characterizing cancer comprising determining a chance of disease-free survival, and/or monitoring disease progression in said subject. This method can be performed by monitoring the markers of a pan-cancer panel in order to detect if a particular cancer in a particular tissue is still absent or not, or whether the quantitative amount of cancer marker versus tissue marker is changing over the time of an anti-cancer treatment. Usually, the longer the markers of a particular pan-cancer panel are absent or even only partially absent, the higher a chance of disease-free survival will be. Similarly, the method according to the present invention can be used in characterizing cancer comprising determining relapse of the disease after complete resection of the tumor in said subject by identifying tissue markers and cancer markers in said sample that are identical to the removed tumor.
The method according to the present invention can be used in characterizing cancer comprising determining metastatic disease by identifying tissue markers in a particular sample that are foreign to the tissue from which said sample is taken from. A foreign tissue marker indicates that the cells of the sample are derived from a foreign origin, i.e. are stemming from metastases.
The method according to the present invention can be used in an improved method for treatment of a proliferative disease, wherein after the analysis of the markers as described hereinabove, a suitable treatment regimen for said proliferative disease to be treated is selected and applied. As will be readily understood, this method can also be employed in the context of all aspects of the general method according to the present invention as described above, i.e. in connection with these. Another aspect of the present invention is therefore related to an improved method of treatment of a proliferative disease, comprising any of the above methods according to the aspects of the present invention, either alone or in a combination.

Preferred is a method according to the present invention, wherein said proliferative disease is cancer, and in particular selected from soft tissue, skin, leukemia, renal, prostate, brain, bone, blood, lymphoid, stomach, head and neck, colon or breast cancer, preferably prostate or breast cancer.

The four terms that apply to the fields of overall genome-wide analysis of all biological processes are called: Proteomics, Transcriptomics, Epigenomics (or Methylomics) and Genomics. Methods and techniques that can be used for studying expression or studying the modifications responsible for expression on all of these levels are well described in the literature and therefore known to a person skilled in the art. They are described in text books of molecular biology and in a large number of scientific journals.

According to the invention, detecting the presence, absence, abundance and/or expression of one or more marker that is specific for more than one proliferative disease as well as the detection of the presence of the expression of tissue markers comprises detecting the expression of physiological, genetic and/or cellular expression and/or cell count, preferably said detecting the expression comprises detecting the expression of protein, mRNA expression and/or the presence or absence of DNA methylation in one or more of said markers. Particularly, said detecting the expression of protein comprises marker-specific antibodies, ELISA, cell sorting techniques, Western blot, or the detection of labeled protein, and said measuring the mRNA expression comprises detection of labeled mRNA or Northern blot. In general, the expression of a marker, such as a gene, or rather the protein encoded by the gene, can be studied in particular on five different levels: firstly, protein expression levels can be determined directly, secondly, mRNA transcription levels can be determined, thirdly, epigenetic modifications, such as gene's DNA methylation profile or the gene's histone profile; can be analysed, as methylation is often correlated with inhibited protein expression, fourth, the gene itself may be analysed for genetic modifications such as mutations, deletions, polymorphisms etc. influencing the expression of the gene product, and fifth, the expression can be detected indirectly, such as, for example, by a change in the cell count of cells that occurs in response to a change in the presence, absence, abundance and/or expression of said marker for proliferative disease.

To detect the levels of mRNA encoding a marker, a sample is obtained from a patient. Said obtaining of a sample is not meant to be retrieving of a sample, as in performing a biopsy, but rather directed to the availability of an isolated biological material representing a specific tissue, relevant for the intended use. The sample can be a tumour tissue sample from the surgically removed tumour, a biopsy sample as taken by a surgeon and provided to the analyst or a sample of blood, plasma, serum or the like. The sample may be treated to extract the nucleic acids contained therein. The resulting nucleic acid from the sample is subjected to gel electrophoresis or other separation techniques. Detection involves contacting the nucleic acids and in particular the mRNA of the sample with a DNA sequence serving as a probe to form hybrid duplexes. The stringency of hybridisation is determined by a number of factors during hybridisation and during the washing procedure, including temperature, ionic strength, length of time and concentration of formamide. These factors are outlined in, for example, Sambrook et al. (Molecular Cloning: A Laboratory Manual, 2nd ed., 1989). Detection of the resulting duplex is usually accomplished by the use of labelled probes. Alternatively, the probe may be unlabeled, but may be detectable by specific binding with a ligand which is labelled, either directly or indirectly. Suitable labels and methods for labelling probes and ligands are known in the art, and include, for example, radioactive labels which may be incorporated by known methods (e.g., nick translation or kinasing), biotin, fluorescent groups, chemiluminescent groups (e.g., dioxetanes, particularly triggered dioxetanes), enzymes, antibodies, and the like.

In order to increase the sensitivity of the detection in a sample of mRNA encoding a marker, the technique of reverse transcription/polymerisation chain reaction can be used to amplify cDNA transcribed from mRNA encoding said marker. The method of reverse transcription/PCR is well known in the art. The reverse transcription/PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and marker-specific primers. (Belyavsky et al, Nucl Acid Res 17:2919-2932, 1989; Krug and Berger, Methods in Enzymology, Academic Press, N.Y., Vol. 152, pp. 316-325, 1987 which are specifically incorporated by reference)

The analysis of protein expression is prior art. It usually requires an antibody specific for the gene product of interest. Appropriate include but are not limited to ELISA or immunohistochemistry.

Thus, any method known in the art for detecting proteins can be used. Such methods include, but are not limited to immunodiffusion, immunoelectrophoresis, immunochemical methods, binder-ligand assays, immunohistochemical techniques, agglutination and complement assays. (for example see Basic and Clinical Immunology, Sites and Terr, eds., Appleton & Lange, Norwalk, Conn. pp 217-262, 1991 which is incorporated by reference). Preferred are binder-ligand immunoassay methods including reacting antibodies with an epitope or epitopes of the marker and competitively displacing a labelled marker protein or derivative thereof.

Certain embodiments of the present invention comprise the use of antibodies specific to the polypeptide markers. In certain embodiments production of monoclonal or polyclonal antibodies can be induced by the use of the marker polypeptide as antigen. Such antibodies may in turn be used to detect expressed proteins. The levels of such proteins present in the peripheral blood of a patient may be quantified by conventional methods. Antibody-protein binding may be detected and quantified by a variety of means known in the art, such as labelling with fluorescent or radioactive ligands. The invention further comprises kits for performing the above-mentioned procedures, wherein such kits comprise antibodies specific for the marker polypeptides.

Numerous competitive and non-competitive protein binding immunoassays are well known in the art. Antibodies employed in such assays may be unlabeled, for example as used in agglutination tests, or labelled for use a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like. Polyclonal or monoclonal antibodies to markers or an epitope thereof can be made for use in immunoassays by any of a number of methods known in the art. One approach for preparing antibodies to a protein is the selection and preparation of an amino acid sequence of all or part of the protein of a marker, chemically synthesising the sequence and injecting it into an appropriate animal, usually a rabbit or a mouse (Milstein and Kohler Nature 256:495-497, 1975; Gulfre and Milstein, Methods in Enzymology: Immunochemical Techniques 73:1-46, Langone and Banatis eds., Academic Press, 1981 which are incorporated by reference). Methods for preparation of a marker or an epitope thereof include, but are not limited to chemical synthesis, recombinant DNA techniques or isolation from biological samples.

A less established area in this context is the field of epigenomics or epigenetics, i.e. the field concerned with analysis of DNA methylation patterns. Methylation of DNA can play an important role in the control of gene expression in mammalian cells. DNA methyltransferases are involved in DNA methylation and catalyse the transfer of a methyl group from S-adenosylmethionine to cytosine residues to form 5-methylcytosine, a modified base that is found mostly at CpG sites in the genome. The presence of methylated CpG islands in the promoter region of genes can suppress their expression. This process may be due to the presence of 5-methylcytosine, which apparently interferes with the binding of transcription factors or other DNA-binding proteins to block transcription. In different types of tumours, aberrant or accidental methylation of CpG islands in the promoter region has been observed for many cancer-related genes, resulting in the silencing of their expression. Such genes include tumour suppressor genes, genes that suppress metastasis and angiogenesis, and genes that repair DNA (Momparler and Bovenzi (2000) J. Cell Physiol. 183:145-54).

Thus, in another and preferred aspect thereof, the object according to the present invention is solved by a method for diagnosing a proliferative disease in a subject comprising the steps of:

a) providing a biological sample from a subject, said biological sample comprising genomic DNA;
b) detecting the level of DNA methylation in one or more markers and determining therefrom upon the presence or absence of a proliferative disease; and c) detecting the level of methylation of one or more markers and determining therefrom if said one or more cell- and/or tissue-markers are atypically present, absent or present at above normal levels within said sample; and d) determining the presence or absence of a cell proliferative disorder and location thereof, based on the level of DNA methylation as detected in step b) and c). Preferably, step b) further comprises comparing said methylation profile to one or more standard methylation profiles, wherein said standard methylation profiles are selected from the group consisting of methylation profiles of non proliferative disease samples and methylation profiles of proliferative disease samples. More preferably, said detecting the presence or absence of DNA methylation comprises the digestion of said genomic DNA with a methylation-sensitive restriction enzyme, followed by multiplexed amplification of gene-specific DNA fragments with CpG islands.

According to the present invention, preferred is a method, wherein said marker that is specific for more than one proliferative disease is selected from the group consisting the genes according to Table 1 and/or nucleic acid sequences thereof according to any of SEQ ID NO: 100 to 161. According to the present invention, preferred is a method, wherein said tissue- and/or cell-specific marker is selected from the group consisting of nucleic acid sequences according to any of SEQ ID NO: 1 to 99. According to the present invention, further preferred is a method, wherein said tissue- and/or cell-specific marker is selected from the group consisting of nucleic acid sequences according to any of SEQ ID NO: 844 to SEQ ID NO: 1255. According to the present invention, preferred is a method, wherein said proliferative disease is selected from psoriasis or cancer, in particular from soft tissue, skin, leukemia, renal, prostate, brain, bone, blood, lymphoid, stomach, head and neck, colon or breast cancer. Further preferred is a method according to the present invention, wherein said biological sample is a biopsy sample or a blood sample.

Even further preferred is a method according to the present invention, wherein said DNA methylation comprises CpG methylation and/or imprinting. Still further preferred is a method according to the present invention, wherein said proliferative disease is in the early pre-clinical stage exhibiting no clinical symptoms. Still further preferred is a method according to the present invention, wherein said detecting the presence or absence of DNA methylation comprises the digestion of said genomic DNA with a methylation-sensitive restriction enzyme followed by multiplexed amplification of gene-specific DNA fragments with CpG islands.

The disclosed invention provides treated nucleic acids, derived from genomic SEQ ID NO: 1 to SEQ ID NO: 161 and SEQ ID NO: 844 to SEQ ID NO: 1255, wherein the treatment is suitable to convert at least one unmethylated cytosine base of the genomic DNA sequence to uracil or another base that is detectably dissimilar to cytosine in terms of hybridization. The genomic sequences in question may comprise one, or more, consecutive or random methylated CpG positions. Said treatment preferably comprises use of a reagent selected from the group consisting of bisulfite, hydrogen sulfite, disulfite, and combinations thereof. In a preferred embodiment of the invention, the objective comprises analysis of a non-naturally occurring modified nucleic acid comprising a sequence of at least 16 contiguous nucleotide bases in length of a sequence selected from the group consisting of SEQ ID NO: 162 TO SEQ ID NO: 805 and SEQ ID NO: 1256 to SEQ ID NO: 2903, wherein said sequence comprises at least one CpG, TpA or CpA dinucleotide and sequences complementary thereto. The sequences of SEQ ID NO: 162 TO SEQ ID NO: 805 provide non-naturally occurring modified versions of the nucleic acid according to SEQ ID NO: 1 TO SEQ ID NO: 161, SEQ ID NO: 1256 to SEQ ID NO: 2903 provide non-naturally occurring modified versions of the nucleic acid according to SEQ ID NO: 844 TO SEQ ID NO: 1255, wherein the modification of each genomic sequence results in the synthesis of a nucleic acid having a sequence that is unique and distinct from said genomic sequence as follows. For each sense strand genomic DNA, e.g., SEQ ID NO: 1, four converted versions are disclosed. A first version wherein “C” is converted to “T,” but “CpG” remains “CpG” (i.e., corresponds to case where, for the genomic sequence, all “C” residues of CpG dinucleotide sequences are methylated and are thus not converted); a second version discloses the complement of the disclosed genomic DNA sequence (i.e. antisense strand), wherein “C” is converted to “T,” but “CpG” remains “CpG” (i.e., corresponds to case where, for all “C” residues of CpG dinucleotide sequences are methylated and are thus not converted). The ‘upmethylated’ converted sequences of SEQ ID NO: 1 to SEQ ID NO: 161 correspond to SEQ ID NO: 162 to SEQ ID NO: 483. The ‘upmethylated’ converted sequences of SEQ ID NO: 844 to SEQ ID NO: 1255 correspond to SEQ ID NO: 1256 to SEQ ID NO: 2079. A third chemically converted version of each genomic sequences is provided, wherein “C” is converted to “T” for all “C” residues, including those of “CpG” dinucleotide sequences (i.e., corresponds to case where, for the genomic sequences, all “C” residues of CpG dinucleotide sequences are unmethylated); a final chemically converted version of each sequence, discloses the complement of the disclosed genomic DNA sequence (i.e. antisense strand), wherein “C” is converted to “T” for all “C” residues, including those of “CpG” dinucleotide sequences (i.e., corresponds to case where, for the complement (antisense strand) of each genomic sequence, all “C” residues of CpG dinucleotide sequences are unmethylated). The ‘downmethylated’ converted sequences of SEQ ID NO: 1 to SEQ ID NO: 161 correspond to SEQ ID NO: 484 to SEQ ID NO: 805. The ‘downmethylated’ converted sequences of SEQ ID NO: 844 to SEQ ID NO: 1253 correspond to SEQ ID NO: 2080 to SEQ ID NO: 2903.

The described invention further discloses oligonucleotides or oligomers for detecting the cytosine methylation state within pretreated DNA of the markers, according to SEQ ID NO: 162 to SEQ ID NO: 805 and SEQ ID NO: 1256 to SEQ ID NO: 2903. Said oligonucleotides or oligomers comprise a nucleic acid sequence having a length of at least nine (9) nucleotides which hybridise, under moderately stringent or stringent conditions (as defined herein above), to a pretreated nucleic acid sequence according to SEQ ID NO: 162 to SEQ ID NO: 805 and SEQ ID NO: 1256 to SEQ ID NO: 2903 and/or sequences complementary thereto. The hybridising portion of the hybridising nucleic acids is typically at least 9, 15, 20, 25, 30 or 35 nucleotides in length. However, longer molecules have inventive utility, and are thus within the scope of the present invention. Particularly preferred is a nucleic acid molecule that hybridize under moderately stringent and/or stringent hybridization conditions to all or a portion of the sequences SEQ ID NO: 162 to SEQ ID NO: 805 and SEQ ID NO: 1256 to SEQ ID NO: 2903 but not SEQ ID NO: 1 to SEQ ID NO: 161, SEQ ID NO: 844 to SEQ ID NO: 1255 or other human genomic DNA.

Hybridising nucleic acids of the type described herein can be used, for example, as a primer (e.g., a PCR primer), or a diagnostic and/or prognostic probe or primer. Preferably, hybridisation of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO: 162 to SEQ ID NO: 805 or SEQ ID NO: 1256 to SEQ ID NO: 2903, rather than identical, it is useful to first establish the lowest temperature at which only homologous hybridisation occurs with a particular concentration of salt (e.g., SSC or SSPE). Then, assuming that 1% mismatching results in a 1° C. decrease in the Tm, the temperature of the final wash in the hybridisation reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NOs: 162 to 805, include those corresponding to sets of consecutively overlapping oligonucleotides of length X, where the oligonucleotides within each consecutively overlapping set (corresponding to a given X value) are defined as the finite set of Z oligonucleotides from nucleotide positions:
n to (n+(X−1));
where n=1, 2, 3, . . . (Y−(X−1));
where Y equals the length (nucleotides or base pairs) of SEQ ID NO: 1;
where X equals the common length (in nucleotides) of each oligonucleotide in the set (e.g., X=20 for a set of consecutively overlapping 20-mers); and
where the number (Z) of consecutively overlapping oligomers of length X for a given SEQ ID NO of length Y is equal to Y−(X−1). For example Z=1,123−19=1,104 for either sense or antisense sets of SEQ ID NO: 1, where X=20.

Preferably, the set is limited to those oligomers that comprise at least one CpG, Cpa or tpG dinucleotide, wherein ‘Cpa’ is indicating that said Cpa hybridises to a position (tpG) which was a CpG prior to bisulfite conversion and is a TpG now; and wherein ‘tpG’ is indicating that said tpG hybridises to a position (Cpa) which is the complementary to a position (tpG) which was a CpG prior to bisulfite conversion and is a TpG now.

The present invention encompasses, for each of SEQ ID NO: 1 to SEQ ID NO: 161 and or SEQ ID NO: 844 to SEQ ID NO: 1255 after chemical pre-treatment, and SEQ ID NO: 162 to SEQ ID NO: 805 and or SEQ ID NO: 1256 to SEQ ID NO: 2903 (sense and antisense), the use of multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO: 1 to SEQ ID NO: 161 and SEQ ID NO: 844 to SEQ ID NO: 1255 after chemical pre-treatment, and SEQ ID NO: 162 to 805 and SEQ ID NO: 1256 to SEQ ID NO: 2903. Preferably, said oligomers comprise at least one Cp, tpG or Cpa dinucleotide. Thus, in a preferred aspect thereof, the present invention does not relate to oligomers or other nucleic acids that are identical to the chromosomal and chemically untreated DNA sequences of the markers according to SEQ ID NO: 1 to SEQ ID NO: 161 and SEQ ID NO: 844 to SEQ ID NO: 1255.

Particularly preferred oligonucleotides or oligomers used to the present invention are those in which the cytosine of the CpG dinucleotide (or of the corresponding converted TpG or CpA dinucleotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides used in this invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridisation-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridisation-triggered cross-linking agent, transport agent, hybridisation-triggered cleavage agent, etc.

The oligonucleotide may also comprise at least one art-recognised modified sugar and/or base moiety, or may comprise a modified backbone or non-natural internucleoside linkage.

The oligomers used in the present invention are normally used in so called “sets” which contain at least one oligomer for analysis of each of the CpG dinucleotides of a genomic sequence comprising SEQ ID NO: 1 to 161 and SEQ ID NO: 844 to SEQ ID NO: 1255 and sequences complementary thereto or to their corresponding CG, tG or Ca dinucleotide within the pretreated nucleic acids according to SEQ ID NO: 162 to SEQ ID NO: 805 and SEQ ID NO: 1256 to SEQ ID NO: 2903 and sequences complementary thereto, wherein a ‘t’ indicates a nucleotide which converted from a cytosine into a thymine and wherein ‘a’ indicates the complementary nucleotide to such a converted thymine. Preferred is a set which contains at least one oligomer for each of the CpG dinucleotides within the respective marker and it's promoter and regulatory elements in both the pretreated and genomic versions of said gene. However, it is anticipated that for economic or other factors it may be preferable to analyse a limited selection of the CpG dinucleotides within said sequences and the contents of the set of oligonucleotides should be altered accordingly. Therefore, the present invention moreover relates to a set of at least 3 n (oligonucleotides and/or PNA-oligomers) used for detecting the cytosine methylation state in genomic DNA (SEQ ID NO: 1 to SEQ ID NO: 161 and SEQ ID NO: 844 to SEQ ID NO: 1255 and sequences complementary thereto) and sequences complementary thereto). These probes enable the detection of the expression of the markers that are specific for cell proliferative disorders. The set of oligomers may also be used for detecting single nucleotide polymorphisms (SNPs) in genomic DNA (SEQ ID NO: 1 to SEQ ID NO: 161 and SEQ ID NO: 844 to SEQ ID NO: 1255, and sequences complementary thereto).

Moreover, the present invention includes the use of a set of at least two oligonucleotides which can be used as so-called “primer oligonucleotides” for amplifying DNA sequences of one of SEQ ID NO: 1 to SEQ ID NO: 805 and SEQ ID NO: 844 to SEQ ID NO: 2903 and sequences complementary thereto, or segments thereof.

In the case of the sets of oligonucleotides according to the present invention, it is preferred that at least one and more preferably all members of the set of oligonucleotides is bound to a solid phase.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices may also be used.

A further subject matter of the present invention relates to a DNA chip for the analysis of cell proliferative disorders. DNA chips are known, for example, in U.S. Pat. No. 5,837,832.

As above, the present invention includes detecting the presence or absence of DNA methylation in one or more marker gene (i.e. and preferably the promoter and regulatory elements). Most preferably the assay according to the following method is used in order to detect methylation within the markers wherein said methylated nucleic acids are present in a solution further comprising an excess of background DNA, wherein the background DNA is present in between 100 to 1000 times the concentration of the DNA to be detected. Said method comprising contacting a nucleic acid sample obtained from said subject with at least one reagent or a series of reagents, wherein said reagent or series of reagents, distinguishes between methylated and non-methylated CpG dinucleotides within the marker.

Preferably, said method comprises the following steps: In the first step, a sample of the tissue to be analysed is obtained. The source may be any suitable source, preferably, the source of the sample is selected from the group consisting of histological slides, biopsies, paraffin-embedded tissue, bodily fluids, plasma, serum, stool, urine, blood, nipple aspirate and combinations thereof. Preferably, the source is tumour tissue, biopsies, serum, urine, blood or nipple aspirate. The most preferred source, is the tumour sample, surgically removed from the patient or a biopsy sample of said patient.

The DNA is then isolated from the sample. Extraction may be by means that are standard to one skilled in the art, including the use of detergent lysates, sonification and vortexing with glass beads. Once the nucleic acids have been extracted, the genomic double stranded DNA is used in the analysis.

In the second step of the method, the genomic DNA sample is treated in such a manner that cytosine bases which are unmethylated at the 5′-position are converted to uracil, thymine, or another base which is dissimilar to cytosine in terms of hybridisation behaviour. This will be understood as ‘pretreatment’ herein.

The above described pretreatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behaviour. Enclosing the DNA to be analysed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996) is one preferred example how to perform said pretreatment. It is further preferred that the bisulfite treatment is carried out in the presence of a radical scavenger or DNA denaturing agent.

The bisulfite-mediated conversion of the genomic sequences into ‘bisulfite sequences’ may take place in any standard, art-recognized format. This includes, but is not limited to modification within agarose gel or in denaturing solvents. The nucleic acid may be, but is not required to be, concentrated and/or otherwise conditioned before the said nucleic acid sample is pretreated with said agent. The pretreatment with bisulfite can be performed within the sample or after the nucleic acids are isolated. Preferably, pretreatment with bisulfite is performed after DNA isolation, or after isolation and purification of the nucleic acids.

The double-stranded DNA is preferentially denatured prior to pretreatment with bisulfite.

The bisulfite conversion thus consists of two important steps, the sulfonation of the cytosine, and the subsequent deamination thereof. The equilibra of the reaction are on the correct side at two different temperatures for each stage of the reaction. The temperatures and length at which each stage is carried out may be varied according to the specific requirements of the situation.

Preferably, sodium bisulfite is used as described in WO 02/072880. Particularly preferred, is the so called agarose-bead method, wherein the DNA is enclosed in a matrix of agarose, thereby preventing the diffusion and renaturation of the DNA (bisulfite only reacts with single-stranded DNA), and replacing all precipitation and purification steps with fast dialysis (Olek et al., Nucleic Acids Res. 24: 5064-5066, 1996). It is further preferred that the bisulfite pretreatment is carried out in the presence of a radical scavenger or DNA denaturing agent, such as oligoethylenglycoldialkylether or preferably Dioxan. The DNA may then be amplified without need for further purification steps.

Said chemical conversion, however, may also take place in any format standard in the art. This includes, but is not limited to modification within agarose gel, in denaturing solvents or within capillaries.

Generally, the bisulfite pretreatment transforms unmethylated cytosine bases, whereas methylated cytosine bases remain unchanged. In a 100% successful bisulfite pretreatment, a complete conversion of all unmethylated cytosine bases into uracil bases takes place. During subsequent hybridization steps, uracil bases behave as thymine bases, in that they form WatsonCrick base pairs with adenine bases. Only cytosine bases that are located in a CpG position (i.e., in a 5′-CG-3′dinucleotide), are known to be possibly methylated (known to be normally methylatable in vivo). Therefore all other cytosines, not located in a CpG position, are unmethylated and are thus transformed into uracils that will pair with adenine during amplification cycles, and as such will appear as thymine bases in an amplified product (e.g., in a PCR product). Whenever a bisulfite-treated nucleic acid is amplified and/or sequence analyzed, the positions that appear as thymines in the sequence can either indicate a true thymine position or a (transformed or converted) cytosine position. These can only be distinguished by comparing the bisulfite sequence data with the untreated genomic sequence data that is already known.

However, cytosines in CpG positions must be regarded as potentially methylated, more precisely as potentially differentially methylated. Significantly, a 100% cytosine or 100% thymine signal at a CpG position will be rare, because biological samples always contain some kind of background DNA. Therefore, according to the inventive methods, the ratio of thymine to cytosine appearing at a specific CpG position is determined as accurately as possible. This is enabled, for example, by using the sequencing evaluation software tool ESME, which takes into account the falsification or bias of this ratio caused by incomplete conversion (see herein below, and application EP 02 090 203, incorporated herein by reference.

In the third step of the method, fragments of the pretreated DNA are amplified. Wherein the source of the DNA is free DNA from serum, or DNA extracted from paraffin it is particularly preferred that the size of the amplificate fragment is between 100 and 200 base pairs in length, and wherein said DNA source is extracted from cellular sources (e.g. tissues, biopsies, cell lines) it is preferred that the amplificate is between 100 and 350 base pairs in length. It is particularly preferred that said amplificates comprise at least one 20 base pair sequence comprising at least three CpG dinucleotides. Said amplification is carried out using sets of primer oligonucleotides according to the present invention, and a preferably heat-stable polymerase. The amplification of several DNA segments can be carried out simultaneously in one and the same reaction vessel, in one embodiment of the method preferably six or more fragments are amplified simultaneously. Typically, the amplification is carried out using a polymerase chain reaction (PCR) and a set of primer oligonucleotides that includes at least two oligonucleotides whose sequences are each reverse complementary, identical, or hybridise under stringent or highly stringent conditions to an at least 18-base-pair long segment of the base sequences of SEQ ID NO: 1 to SEQ ID NO: 161 and SEQ ID NO: 844 to SEQ ID NO: 1255 after chemical pre-treatment, and SEQ ID NO: 162 to SEQ ID NO: 805 and SEQ ID NO: 1256 to SEQ ID NO: 2903 and sequences complementary thereto.

In an alternate embodiment of the method, the methylation status of preselected CpG positions within the nucleic acid sequences comprising SEQ ID NO: 1 to SEQ ID NO: 161 and SEQ ID NO: 844 to SEQ ID NO: 1255 after methylation specific conversion may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridises to a bisulfite treated CpG dinucleotide. Therefore, the sequence of said primers comprises at least one CpG, TpG or CpA dinucleotide. MSP primers specific for non-methylated DNA contain a “T” at the 3′ position of the C position in the CpG. Preferably, therefore, the base sequence of said primers is required to comprise a sequence having a length of at least 18 nucleotides which hybridises to a pretreated nucleic acid sequence according to SEQ ID NO: 162 to SEQ ID NO: 805 and SEQ ID NO: 1256 to SEQ ID NO: 2903 and sequences complementary thereto, wherein the base sequence of said oligomers comprises at least one CpG, tpG or Cpa dinucleotide. In this embodiment of the method according to the invention it is particularly preferred that the MSP primers comprise between 2 and 4 CpG, tpG or Cpa dinucleotides. It is further preferred that said dinucleotides are located within the 3′ half of the primer e.g. wherein a primer is 18 bases in length the specified dinucleotides are located within the first 9 bases form the 3′ end of the molecule. In addition to the CpG, tpG or Cpa dinucleotides it is further preferred that said primers should further comprise several bisulfite converted bases (i.e. cytosine converted to thymine, or on the hybridising strand, guanine converted to adenosine). In a further preferred embodiment said primers are designed so as to comprise no more than 2 cytosine or guanine bases.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labelled amplificates have a single positive or negative net charge, allowing for better detectability in the mass spectrometer. The detection may be carried out and visualised by means of, e.g., matrix assisted laser desorption/ionisation mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionisation process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In a particularly preferred embodiment of the method the amplification of step three is carried out in the presence of at least one species of blocker oligonucleotides. The use of such blocker oligonucleotides has been described by Yu et al., BioTechniques 23:714-720, 1997. The use of blocking oligonucleotides enables the improved specificity of the amplification of a subpopulation of nucleic acids. Blocking probes hybridised to a nucleic acid suppress, or hinder the polymerase mediated amplification of said nucleic acid. In one embodiment of the method blocking oligonucleotides are designed so as to hybridise to background DNA. In a further embodiment of the method said oligonucleotides are designed so as to hinder or suppress the amplification of unmethylated nucleic acids as opposed to methylated nucleic acids or vice versa.

Blocking probe oligonucleotides are hybridised to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridise to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘TpG’ at the position in question, as opposed to a ‘CpG.’ In one embodiment of the method the sequence of said blocking oligonucleotides should be identical or complementary to molecule is complementary or identical to a sequence at least 18 base pairs in length selected from the group consisting of SEQ ID NO: 1 to SEQ ID NO: 161 and SEQ ID NO: 844 to SEQ ID NO: 1255 after chemical pre-treatment, and SEQ ID NO: 162 to SEQ ID NO: 805 and SEQ ID NO: 1256 to SEQ ID NO: 2903, preferably comprising one or more CpG, TpG or CpA dinucleotides.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatised at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-termini thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridised blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

In one embodiment of the method, the binding site of the blocking oligonucleotide is identical to, or overlaps with that of the primer and thereby hinders the hybridisation of the primer to its binding site. In a further preferred embodiment of the method, two or more such blocking oligonucleotides are used. In a particularly preferred embodiment, the hybridisation of one of the blocking oligonucleotides hinders the hybridisation of a forward primer, and the hybridisation of another of the probe (blocker) oligonucleotides hinders the hybridisation of a reverse primer that binds to the amplificate product of said forward primer.

In an alternative embodiment of the method, the blocking oligonucleotide hybridises to a location between the reverse and forward primer positions of the treated background DNA, thereby hindering the elongation of the primer oligonucleotides.

It is particularly preferred that the blocking oligonucleotides are present in at least 5 times the concentration of the primers.

In the fourth step of the method, the amplificates obtained during the third step of the method are analysed in order to ascertain the methylation status of the CpG dinucleotides prior to the treatment.

In embodiments where the amplificates were obtained by means of MSP amplification and/or blocking oligonucleotides, the presence or absence of an amplificate is in itself indicative of the methylation state of the CpG positions covered by the primers and or blocking oligonucleotide, according to the base sequences thereof. All possible known molecular biological methods may be used for this detection, including, but not limited to gel electrophoresis, sequencing, liquid chromatography, hybridisations, real time PCR analysis or combinations thereof. This step of the method further acts as a qualitative control of the preceding steps.

In the fourth step of the method amplificates obtained by means of both standard and methylation specific PCR are further analysed in order to determine the CpG methylation status of the genomic DNA isolated in the first step of the method. This may be carried out by means of hybridisation-based methods such as, but not limited to, array technology and probe based technologies as well as by means of techniques such as sequencing and template directed extension.

In one embodiment of the method, the amplificates synthesised in step three are subsequently hybridised to an array or a set of oligonucleotides and/or PNA probes. In this context, the hybridisation takes place in the following manner: the set of probes used during the hybridisation is preferably composed of at least two oligonucleotides or PNA-oligomers; in the process, the amplificates serve as probes which hybridise to oligonucleotides previously bonded to a solid phase; the non-hybridised fragments are subsequently removed; said oligonucleotides contain at least one base sequence having a length of at least 9 nucleotides which is reverse complementary or identical to a segment of the base sequences specified in the SEQ ID NO: 1 to SEQ ID NO: 161 and SEQ ID NO: 844 to SEQ ID NO: 1255 after chemical pre-treatment, and SEQ ID NO: 162 to SEQ ID NO: 805 and SEQ ID NO: 1256 to SEQ ID NO: 2903; and the segment comprises at least one CpG, TpG or CpA dinucleotide.

In a preferred embodiment, said dinucleotide is present in the central third of the oligomer. Said oligonucleotide may also be present in the form of peptide nucleic acids. The non-hybridised amplificates are then removed. The hybridised amplificates are detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes that are hybridised to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393). There are two preferred embodiments of utilising this method. One embodiment, known as the TaqMan™ assay employs a dual-labelled fluorescent oligonucleotide probe. The TaqMan™ PCR reaction employs the use of a non-extendible interrogating oligonucleotide, called a TaqMan™ probe, which is designed to hybridise to a CpG-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. Hybridised probes are displaced and broken down by the polymerase of the amplification reaction thereby leading to an increase in fluorescence. For analysis of methylation within nucleic acids subsequent to bisulfite treatment, it is required that the probe be methylation specific, as described in U.S. Pat. No. 6,331,393, (hereby incorporated by reference in its entirety) also known as the MethyLight assay. The second preferred embodiment of this MethyLight technology is the use of dual-probe technology (Lightcycler®), each probe carrying donor or recipient fluorescent moieties, hybridisation of two probes in proximity to each other is indicated by an increase or fluorescent amplification primers. Both these techniques may be adapted in a manner suitable for use with bisulfite treated DNA, and moreover for methylation analysis within CpG dinucleotides.

Also any combination of these probes or combinations of these probes with other known probes may be used.

In a further preferred embodiment of the method, the fourth step of the method comprises the use of template-directed oligonucleotide extension, such as MS-SNuPE as described by Gonzalgo & Jones, Nucleic Acids Res. 25:2529-2531, 1997. In said embodiment it is preferred that the methylation specific single nucleotide extension primer (MS-SNuPE primer) is identical or complementary to a sequence at least nine but preferably no more than twenty five nucleotides in length of one or more of the sequences taken from the group of SEQ ID NO: 1 to SEQ ID NO: 161 and SEQ ID NO: 844 to SEQ ID NO: 1255 after chemical pre-treatment, and SEQ ID NO: 162 to SEQ ID NO: 805 and SEQ ID NO: 1256 to SEQ ID NO: 2903. However it is preferred to use fluorescently labelled nucleotides, instead of radiolabelled nucleotides.

In yet a further embodiment of the method, the fourth step of the method comprises sequencing and subsequent sequence analysis of the amplificate generated in the third step of the method (Sanger F., et al., Proc Natl Acad Sci USA 74:5463-5467, 1977).

Additional embodiments of the invention provide a method for the analysis of the methylation status of genomic DNA according to the markers used in the invention without the need for pretreatment.

In the first step of such additional embodiments, the genomic DNA sample is isolated from tissue or cellular sources. Preferably, such sources include cell lines, histological slides, biopsy tissue, body fluids, or breast tumour tissue embedded in paraffin. Extraction may be by means that are standard to one skilled in the art, including but not limited to the use of detergent lysates, sonification and vortexing with glass beads. Once the nucleic acids have been extracted, the genomic double-stranded DNA is used in the analysis.

In a preferred embodiment, the DNA may be cleaved prior to the treatment, and this may be by any means standard in the state of the art, but preferably with methylation-sensitive restriction endonucleases.

In the second step, the DNA is then digested with one or more methylation sensitive restriction enzymes. The digestion is carried out such that hydrolysis of the DNA at the restriction site is informative of the methylation status of a specific CpG dinucleotide.

In the third step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels.

In the final step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis.

In yet another preferred aspect thereof, the object according to the present invention is solved by a method for generating a pan-cancer marker panel of proliferative disease markers and, in particular pan-cancer markers, together with tissue- and/or cell-specific markers for the improved diagnosis of a proliferative disease in a subject. The method comprises a) providing a biological sample from said subject suspected of or previously being diagnosed as having a proliferative disease, b) providing a first set of one or more markers indicative for proliferative disease (e.g. pan-cancer markers), c) determining the presence, absence, abundance and/or expression of said one or more markers of step b); d) providing a first set of cell- and/or tissue markers, e) determining the expression of said one or more markers of step d), and f) generating a pan-cancer marker panel of proliferative disease markers and, in particular pan-cancer markers being specific for said proliferative disease in said subject by selecting those tissue- and/or cell-specific markers and proliferative disease markers and, in particular pan-cancer markers that are differently present, absent, abundant and/or expressed in said subject when compared to a respective profile of a non proliferative-disease (e.g. non-cancerous) sample. In one particularly preferred embodiment of the method, said marker is indicative for more than one proliferative disease. Preferably, said biological sample is a biopsy sample or a blood sample.

Preferred is a method, wherein said detecting the expression of one or more markers comprises measuring cell count, the expression of protein, mRNA expression and/or the presence or absence or the level of DNA methylation in one or more of said markers. According to a preferred aspect of the inventive method, the markers of step b) are selected from the group consisting of nucleic acid sequences according to any of SEQ ID NO: 100 to SEQ ID NO: 161, whilst the tissue- and/or cell-specific markers of step c) are selected from the group consisting of nucleic acid sequences according to any of SEQ ID NO: 1 to SEQ ID NO: 99, or more preferably from the group consisting SEQ ID NO: 844 to SEQ ID NO: 1255. Thus, in preferred embodiments of the inventive method, these sets or groups of markers form the basis for particular sets of markers that are actually selected into a panel.

Further preferred is a method, wherein said measuring the expression of protein comprises marker-specific antibodies, ELISA, cell sorting techniques, Western blot, mRNA expression or the detection of labeled protein. In another preferred embodiment of the method, said measuring the mRNA expression comprises detection of labeled mRNA or Northern blot. Further preferred is a method, wherein said detecting of the expression is qualitative or additionally quantitative.

As a non-limiting but preferred example, for the actual generation of a marker panel of proliferative disease markers, first, a database or other type of listing of a set of one or more of the proliferative disease markers, e.g. all of those as given herein, is generated. Then, the expression of these markers is detected in a sample that is taken from the subject suspected of having a proliferative disease or being diagnosed with suffering from a particular proliferative disease. Detecting the expression of said one or more markers indicative for proliferative disease can be performed as described above and can comprise measuring the expression of protein, mRNA expression and/or the presence or absence of DNA methylation in one or more of said markers. In one embodiment, this analysis is then compared with the result(s) of an expression profile of a non proliferative-disease (e.g. non-cancerous) sample (in the following, “blank-sample”), in other embodiments, this comparison is performed after the subsequent analysis of the cell- and/or tissue-markers. For statistical reasons, the comparison can also be done with several analyses in parallel using sample derived either from the same patient or other non-diseased patients.

In one preferred embodiment, markers that differ in their expression (i.e. are expressed either higher or lower or are present or absent when compared to the blank sample) and/or their level of methylation are then selected into a pan-cancer panel and stored in a database or a listing. This pan-cancer panel can then be used in later diagnoses of similar or identical proliferative diseases in many patients or as a “personalized” pan-cancer panel for an individual patient, e.g. for follow-up analyses.

Further preferred is a method, wherein a pan-cancer panel is selected, whereby the markers are selected from the group consisting of nucleic acid sequences according to any of SEQ ID NO: 100 to SEQ ID NO: 161 and wherein at least one (more preferably a plurality) marker is selected from the group consisting of nucleic acid sequences according to any of SEQ ID NO: 1 to SEQ ID NO: 99 or more preferably SEQ ID NO: 844 to SEQ ID NO: 1255.

Preferred is a selection into a pan-cancer panel, wherein the proliferative disease is selected from soft tissue, skin, leukemia, renal, prostate, brain, bone, blood, lymphoid, stomach, head and neck, colon or breast cancer.

Further preferred is a method, wherein said DNA methylation that is detected and/or analyzed comprises CpG methylation and/or imprinting. In another aspect of the method according to the present invention, said detecting the presence or absence of DNA methylation comprises the digestion of said genomic DNA with a methylation-sensitive restriction enzyme followed by multiplexed amplification of gene-specific DNA fragments with CpG islands.

Further preferred is a method, wherein said proliferative disease is in the early pre-clinical stage exhibiting no clinical symptoms, i.e. in cases, where a common physiological diagnosis, such as a visual diagnosis or inspection, would not detect an existing proliferative disease.

Another aspect of the method according to the present invention then relates to an improved method for the treatment of a proliferative disease, comprising a method as described above, and selecting a suitable treatment regimen for said proliferative disease to be treated. The treatment regimen can also be adapted to the changes in said proliferative disease status of the patient that have been identified using the method according to the invention. The selection or adaptation is commonly made by the attending physician and can include further clinical parameters that are related to the disease and/or the patient(s) to be treated. Preferably, said proliferative disease is cancer.

In another aspect of the present invention, the methods of the invention can be performed manually or partially or fully automated, such as on a computer and/or a suitable robot. Accordingly, also encompassed by the present invention is a suitable computer program product, e.g. a software, for performing the method according to the present invention when run on a computer, which can be present on a suitable data carrier.

In one embodiment of the method according to the invention, the generating a pan-cancer marker panel comprises the use of ESME. ESME calculates methylation levels at particular CpG positions by comparing signal intensities, and correcting for incomplete bisulphite conversion. ESME scores all cytosines (=methylated C) and C to T transitions (=non-methylated C) in bisulphite sequence traces, and furthermore calculates the % of methylation for all CpG sites. It allows the analysis of DNA mixtures both in individual cells as well as of DNA mixtures from a plurality of cells. The method can be applied to any bisulfite-pretreated nucleic acid for which the genomic nucleotide sequence of the corresponding DNA region not treated with bisulfite is known, and for which a sequence electropherogram (trace) can also be generated.

ESME utilizes the electropherograms for standardizing the average signal intensity of at least one base type (C, T, A or G) against the average signal intensity which is obtained for one or more of the remaining base types. Preferably, the cytosine signal intensities are standardized relative to the thymine signal intensities, and the ratio of the average signal intensity of cytosine to that of thymine is determined.

The average of a signal intensity is calculated by taking into account the signal intensities of several bases, which are present in a randomly defined region of the amplificate. The average of a plurality of positions of this base type is determined within an arbitrarily defined region of the amplificate. This region can comprise the entire amplificate, or a portion thereof. Significantly, such averaging leads to mathematically reasonable and/or statistically reliable values.

Additionally, a basic feature of ESME comprises calculation of a ‘conversion rate’ (fcon) of the conversion of cytosine to uracil (as a consequence of bisulfite treatment), based upon the standardized signal intensities. This is characterized as the ratio of at least one signal intensity standardized at positions which modify their hybridization behaviour due to the pretreatment, to at least one other signal intensity. Preferably, it is the ratio of unmethylated cytosine bases, whose hybridization behaviour was modified (into the hybridization behaviour of thymine) by bisulfite treatment, to all unmethylated cytosine bases, independent of whether their hybridization behaviour was modified or not, within a defined sequence region. The region to be considered can comprise the length of the total amplificate, or only a part of it, and both the sense sequence or its inversely-complementary sequence can be utilized therefore.

The calculation of standardizing factors, for standardizing signal intensities, as well as the calculation of a conversion rate are based on accurate knowledge of signal intensities. Preferably, such knowledge is as accurate as possible. An electropherogram represents a curve that reflects the number of detected signals per unit of time, which in turn reflects the spatial distance between two bases (as an inherent characteristic of the sequencing method). Therefore, the signal intensity and thus the number of molecules that bear that signal can be calculated by the area under the peak (i.e., under the local maximum of this curve). The considered area is best described by integrating this curve. Such area measurements are determined by the integration limits X1 and X2; X1, lying to the left of the local maximum, and by X2, lying to the right of the local maximum. Another basic feature of ESME is that it affords the determination of the actual methylation number fMET, (“actual” as in significantly closer to reality than assuming the conversion rate is, e.g., 95%). Both, the standardized signal intensities as well as the conversion rates fcon (obtained by considering said standardized signal intensities) are used for calculation of the actual degree (level) of methylation of a cytosine position in question.

According to a preferred embodiment, the % methylation levels are calculated by ESME, or an equivalent thereof, for all CpG positions representing the genome, and the information is linked to corresponding positions in the latest assembly of the human genome sequence, and be sorted according to tissue and disease state. In preferred embodiments, this information is made available for further research. In a particularly preferred embodiment, the information is utilized directly to provide specific markers for DNA derived from specific cell or tissue types.

The methylation data, including the quantitative aspects thereof, is easily presented in a user friendly two-dimensional display, allowing for immediate identification of differentiating patterns. For example, the location of a CpG position within the genome is displayed along one axis, whereas the sample type is displayed along the other axis. When grouping the phenotypically distinct sample types side-by-side, methylation differences can be displayed in the field created by the two axes.

An additional aspect of the present invention is a kit for diagnosing a proliferative disease in a subject, comprising reagents for detecting the expression of one or more proliferative disease markers; and reagents for localizing the proliferative disease and/or characterizing the type of proliferative disease by detecting specific cell- and/or tissue-markers based on nucleic acid-analysis. Preferably, the kit further comprising instructions for using said kit for characterizing cancer in said subject, as detailed below. Preferably, said reagents comprise reagents for detecting the presence or absence of DNA methylation in markers, as also detailed below. Further preferred is a kit according to the present invention, wherein the markers are selected from the group consisting of nucleic acid sequences according to any of SEQ ID NO: 1 to SEQ ID NO: 161 or SEQ ID NO: 844 to SEQ ID NO: 2903, and chemically pretreated sequences thereof.

A representative kit may comprise one or more nucleic acid segments as described above that selectively hybridise to marker mRNA and a container for each of the one or more nucleic acid segments. In certain embodiments the nucleic acid segments may be combined in a single tube. In further embodiments, the nucleic acid segments may also include a pair of primers for amplifying the target mRNA. Such kits may also include any buffers, solutions, solvents, enzymes, nucleotides, or other components for hybridisation, amplification or detection reactions. Preferred kit components include reagents for reverse transcription-PCR, in situ hybridisation, Northern analysis and/or RPA.

Said kit may further comprise instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight™, HeavyMethyl™, COBRA, and nucleic acid sequencing. However, a kit along the lines of the present invention can also contain only part of the aforementioned components.

Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridisation oligo; control hybridisation oligo; kinase labelling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Typical reagents (e.g., as might be found in a typical MethyLight®-based kit) for MethyLight® analysis may include, but are not limited to: PCR primers for specific gene (or methylation-altered DNA sequence or CpG island); TaqMan® probes; optimised PCR buffers and deoxynucleotides; and Taq polymerase.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimised PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

Typical reagents (e.g., as might be found in a typical MSP-based kit) for MSP analysis may include, but are not limited to: methylated and unmethylated PCR primers for specific gene (or methylation-altered DNA sequence or CpG island), optimised PCR buffers and deoxynucleotides, and specific probes.

It should be understood that the features of the invention as disclosed and described herein can be used not only in the respective combination as indicated but also in a singular fashion without departing from the intended scope of the present invention.

The invention will now be described in more detail by reference to the following Sequence listing, and the Examples. The following examples are provided for illustrative purposes only and are not intended to limit the invention.

TABLE 1
Proliferative disease markers according to the present invention

| | | Methylated | Methylated | | Unmethylated |
|---|---|---|---|---|---|
| | Genomic | converted | converted | Unmethylated | converted |
| | sequence | sense | antisense | converted | antisense |
| | SEQ ID | strand SEQ | strand SEQ | sense strand | strand SEQ |
| Gene name | NO: | ID NO: | ID NO: | SEQ ID NO: | ID NO: |
| VIAAT | 100 | 360 | 361 | 682 | 683 |
| HS3ST2 | 101 | 362 | 363 | 684 | 685 |
| UCN | 102 | 364 | 365 | 686 | 687 |
| TMEFF2 | 103 | 366 | 367 | 688 | 689 |
| Not applicable | 104 | 368 | 369 | 690 | 691 |
| Not applicable | 105 | 370 | 371 | 692 | 693 |
| SIX6 | 106 | 372 | 373 | 694 | 695 |
| LIM/HOMEOBOX | | | | | |
| PROTEIN LHX9 | 107 | 374 | 375 | 696 | 697 |
| Not applicable | 108 | 376 | 377 | 698 | 699 |
| PROSTAGLANDIN | | | | | |
| E2 RECEPTOR | 109 | 378 | 379 | 700 | 701 |
| ORPHAN | | | | | |
| NUCLEAR | | | | | |
| RECEPTOR NR5A2 | 110 | 380 | 381 | 702 | 703 |
| HOMEOBOX | | | | | |
| PROTEIN GSH-2 | 111 | 382 | 383 | 704 | 705 |
| HISTONE H4 | 112 | 384 | 385 | 706 | 707 |
| Not applicable | 113 | 386 | 387 | 708 | 709 |
| MUC5B | 114 | 388 | 389 | 710 | 711 |
| SASH1 | 115 | 390 | 391 | 712 | 713 |
| S100A7 | 116 | 392 | 393 | 714 | 715 |
| BCL11B | 117 | 394 | 395 | 716 | 717 |
| Not applicable | 118 | 396 | 397 | 718 | 719 |
| MGC34831 | 119 | 398 | 399 | 720 | 721 |
| Not applicable | 120 | 400 | 401 | 722 | 723 |
| Not applicable | 121 | 402 | 403 | 724 | 725 |
| Not applicable | 122 | 404 | 405 | 726 | 727 |
| Not applicable | 123 | 406 | 407 | 728 | 729 |
| PRDM6 | 124 | 408 | 409 | 730 | 731 |
| DKK3 | 125 | 410 | 411 | 732 | 733 |
| GIRK2 | 126 | 412 | 413 | 734 | 735 |
| Not applicable | 127 | 414 | 415 | 736 | 737 |
| Not applicable | 128 | 416 | 417 | 738 | 739 |
| Not applicable | 129 | 418 | 419 | 740 | 741 |
| GS1 | 130 | 420 | 421 | 742 | 743 |
| Not applicable | 131 | 422 | 423 | 744 | 745 |
| DDX51 | 132 | 424 | 425 | 746 | 747 |
| Not applicable | 133 | 426 | 427 | 748 | 749 |
| Not applicable | 134 | 428 | 429 | 750 | 751 |
| Not applicable | 135 | 430 | 431 | 752 | 753 |
| APC | 136 | 432 | 433 | 754 | 755 |
| CDKN2A | 137 | 434 | 435 | 756 | 757 |
| CD44 | 138 | 436 | 437 | 758 | 759 |
| DAPK1 | 139 | 438 | 439 | 760 | 761 |
| EYA4 | 140 | 440 | 441 | 762 | 763 |
| GSTP1 | 141 | 442 | 443 | 764 | 765 |
| MLH1 | 142 | 444 | 445 | 766 | 767 |
| PGR | 143 | 446 | 447 | 768 | 769 |
| SERPINB5 | 144 | 448 | 449 | 770 | 771 |
| RARB | 145 | 450 | 451 | 772 | 773 |
| SOD2 | 146 | 452 | 453 | 774 | 775 |
| TERT | 147 | 454 | 455 | 776 | 777 |
| TGFBR2 | 148 | 456 | 457 | 778 | 779 |
| TP73 | 149 | 458 | 459 | 780 | 781 |
| NME1 | 150 | 460 | 461 | 782 | 783 |
| Not applicable | 151 | 462 | 463 | 784 | 785 |
| ESR1 | 152 | 464 | 465 | 786 | 787 |
| CASP8 | 153 | 466 | 467 | 788 | 789 |
| FABP3 | 154 | 468 | 469 | 790 | 791 |
| RARA | 155 | 470 | 471 | 792 | 793 |
| ESR2 | 156 | 472 | 473 | 794 | 795 |
| Not applicable | 157 | 474 | 475 | 796 | 797 |
| SNCG | 158 | 476 | 477 | 798 | 799 |
| SLC19A1 | 159 | 478 | 479 | 800 | 801 |
| GJB2 | 160 | 480 | 481 | 802 | 803 |
| MCT1 | 161 | 482 | 483 | 804 | 805 |

TABLE 2
Tissue/cell specific markers according to the present invention

| | | | | Unmethylated | | | | |
|---|---|---|---|---|---|---|---|---|
| Genomic | Methylated converted | Methylated converted | Unmethylated | converted | | | | |
| sequence | sense | antisense | converted | antisense | | | | |
| SEQ ID | strand SEQ | strand SEQ | sense strand | strand SEQ | | | | |
| NO: | ID NO: | ID NO: | SEQ ID NO: | ID NO: | Gene name | | Ensembl ID | Methylation profile |
| 1 | 162 | 163 | 484 | 485 | SLC7A4 | solute carrier family 7 | ENSG00000099960 | Methylated in Melanocytes |
| | | | | | | (cationic amino acid | | |
| | | | | | | transporter, y+ system), | | |
| | | | | | | member 4 | | |
| 2 | 164 | 165 | 486 | 487 | CTA-373H7.4 | | OTTHUMG00000030780 | Methylated in CD4/CD8 |
| 3 | 166 | 167 | 488 | 489 | RP1-47A17.8 | | OTTHUMG00000030878 | Unmethylated in fibroblasts |
| 4 | 168 | 169 | 490 | 491 | RP4-539M6.7 | | OTTHUMG00000030918 | Unmethylated in |
| | | | | | | | | Keratinocyctes |
| 5 | 170 | 171 | 492 | 493 | CTA-243E7.3 | | OTTHUMG00000030167 | Methylated in Melanocytes |
| 6 | 172 | 173 | 494 | 495 | OSM | Oncostatin M | ENSG00000099985 | Unmethylated in CD4/CD8 |
| 7 | 174 | 175 | 496 | 497 | CTA-299D3.6 | | OTTHUMG00000030140 | Unmethylated in Melanocytes |
| 8 | 176 | 177 | 498 | 499 | CTA-941F9.6 | | OTTHUMG00000030231 | Unmethylated in |
| | | | | | | | | Keratinocyctes |
| 9 | 178 | 179 | 500 | 501 | SUSD2 | | ENSG00000099994 | Methylated in CD4/CD8 |
| 10 | 180 | 181 | 502 | 503 | CTA-503F6.1 | | OTTHUMG00000030870 | Methylated in CD4/CD8 |
| 11 | 182 | 183 | 504 | 505 | PIK4CA | Phosphatidylinositol 4- | ENSG00000133511 | Methylated in CD4/CD8 |
| | | | | | | kinase alpha (EC 2.7.1.67) | | |
| | | | | | | (PI4-kinase) (PtdIns-4- | | |
| | | | | | | kinase) (PI4K-alpha). | | |
| 12 | 184 | 185 | 506 | 507 | A4GALT | Lactosylceramide 4-alpha- | ENSG00000128274 | Methylated in CD4/CD8 |
| | | | | | | galactosyltransferase (EC | | |
| | | | | | | 2.4.1.228) | | |
| 13 | 186 | 187 | 508 | 509 | Q7Z2M6_HUMAN | | ENSG00000188078 | Methylated in CD4/CD8 |
| 14 | 188 | 189 | 510 | 511 | SS3R | Somatostatin receptor type 3 | ENSG00000183473 | Methylated in CD4/CD8 |
| 15 | 190 | 191 | 512 | 513 | GAR22/GAS2L1 | GAS-2 related protein on | ENSG00000185340 | Unmethylated in Melanocytes |
| | | | | | | chromosome 22 (GAR22 | | |
| | | | | | | protein) | | |
| 16 | 192 | 193 | 514 | 515 | BAIAP2L2 | BAI1-associated protein 2- | ENSG00000128298 | Methylated in CD4/CD8 |
| | | | | | | like 2 | | |
| 17 | 194 | 195 | 516 | 517 | SOX10 | SRY (sex determining | OTTHUMG00000030073 | Unmethylated in Melanocytes |
| | | | | | | region Y)-box 10 | | |
| 18 | 196 | 197 | 518 | 519 | PARVG | Gamma-parvin. | ENSG00000138964 | Unmethylated in CD4/CD8 |
| 19 | 198 | 199 | 520 | 521 | CELSR1 | cadherin, EGF LAG seven- | OTTHUMG00000030722 | Unmethylated in CD4/CD8 |
| | | | | | | pass G-type receptor 1 | | |
| 20 | 200 | 201 | 522 | 523 | SMTN | Smoothelin | ENSG00000183963 | Unmethylated in fibroblasts |
| 21 | 202 | 203 | 524 | 525 | GRAP2 | GRB2-related adaptor | OTTHUMG00000030700 | Unmethylated in |
| | | | | | | protein 2 | | Keratinocyctes |
| 22 | 204 | 205 | 526 | 527 | NP_073622.2 ( | CAP-binding protein | ENSG00000186976 | Unmethylated in |
| | | | | | | complex interacting protein | | Keratinocyctes |
| | | | | | | 1 isoform a | | |
| 23 | 206 | 207 | 528 | 529 | SAM50_HUMAN | SAM50-like protein CGI- | ENSG00000100347 | Unmethylated in Cd4/CD8 |
| | | | | | | 51 | | |
| 24 | 208 | 209 | 530 | 531 | RP3-509I19.3 | | OTTHUMG00000015679 | Keratinocyctes |
| 26 | 212 | 213 | 534 | 535 | | | | Unmethylated in fibroblasts |
| 27 | 214 | 215 | 536 | 537 | MOG | Myelin-oligodendrocyte | ENSG00000137345 | Unmethylated in |
| | | | | | | glycoprotein precursor. | | Keratinocyctes |
| 28 | 216 | 217 | 538 | 539 | RP11-417E7.1 | | OTTHUMG00000016054 | Unmethylated in fibroblasts |
| 29 | 218 | 219 | 540 | 541 | CMAH/ | cytidine monophosphate- | OTTHUMG00000016099/ | Unmethylated in |
| | | | | | RP11-191A15.4 | N-acetylneuraminic acid | OTTHUMG00000014386 | Keratinocyctes |
| | | | | | | hydroxylase (CMP-N- | | |
| | | | | | | acetylneuraminate | | |
| | | | | | | monooxygenase) | | |
| 30 | 220 | 221 | 542 | 543 | PKHD1 | Polycystic kidney and | ENSG00000170927 | Unmethylated in |
| | | | | | | hepatic disease 1 precursor | | Keratinocyctes |
| | | | | | | (Fibrocystin) (Polyductin) | | |
| | | | | | | (Tigmin | | |
| 31 | 222 | 223 | 544 | 545 | RP11-411K7.1 | | OTTHUMG00000014887 | Unmethylated in |
| | | | | | | | | Keratinocyctes |
| 32 | 224 | 225 | 546 | 547 | SLC22A1 | solute carrier family 22 | OTTHUMG00000015947 | Unmethylated in liver |
| | | | | | | (organic cation | | |
| | | | | | | transporter), member 1 | | |
| 33 | 226 | 227 | 548 | 549 | PLG | Plasminogen precursor (EC | ENSG00000122194 | Unmethylated in liver |
| | | | | | | 3.4.21.7) [Contains: | | |
| | | | | | | Angiostatin] | | |
| 34 | 228 | 229 | 550 | 551 | RP1-32B1.4 | | OTTHUMG00000015628 | Unmethylated in |
| | | | | | | | | Keratinocyctes |
| 35 | 230 | 231 | 552 | 553 | RP11-203H2.1 | | OTTHUMG00000014222 | Unmethylated in |
| | | | | | | | | Keratinocyctes |
| 36 | 232 | 233 | 554 | 555 | TGM3 | Protein-glutamine | ENSG00000125780 | Unmethylated in |
| | | | | | | glutamyltransferase E | | Keratinocyctes |
| | | | | | | precurso | | |
| 37 | 234 | 235 | 556 | 557 | RASSF2 | Ras association | OTTHUMG00000031790 | Unmethylated in fibroblasts |
| | | | | | | (RalGDS/AF-6) domain | | |
| | | | | | | family 2 | | |
| 38 | 236 | 237 | 558 | 559 | | | | Unmethylated in fibroblasts |
| 39 | 238 | 239 | 560 | 561 | | | | Methylated in CD4/CD8 |
| 40 | 240 | 241 | 562 | 563 | | | | Unmethylated in |
| | | | | | | | | Keratinocyctes |
| 41 | 242 | 243 | 564 | 565 | | | | Unmethylated in CD4/CD8 |
| 42 | 244 | 245 | 566 | 567 | | | | Unmethylated in fibroblasts |
| 43 | 246 | 247 | 568 | 569 | | | | Unmethylated in |
| | | | | | | | | Keratinocyctes |
| 44 | 248 | 249 | 570 | 571 | | | | Unmethylated in fibroblasts |
| 45 | 250 | 251 | 572 | 573 | | | | Unmethylated in |
| | | | | | | | | Keratinocyctes |
| 46 | 252 | 253 | 574 | 575 | | | | Unmethylated in |
| | | | | | | | | Keratinocyctes |
| 47 | 254 | 255 | 576 | 577 | | | | Unmethylated in CD4/CD8 |
| 48 | 256 | 257 | 578 | 579 | | | | Unmethylated in |
| | | | | | | | | Keratinocyctes |
| 49 | 258 | 259 | 580 | 581 | | | | Unmethylated in fibroblasts |
| 50 | 260 | 261 | 582 | 583 | | | | Unmethylated in fibroblasts |
| 51 | 262 | 263 | 584 | 585 | | | | Unmethylated in heart muscle |
| 52 | 264 | 265 | 586 | 587 | | | | Unmethylated in Melanocytes |
| 53 | 266 | 267 | 588 | 589 | | | | Unmethylated in liver |
| 54 | 268 | 269 | 590 | 591 | | | | Methylated in CD4/CD8 |
| 55 | 270 | 271 | 592 | 593 | | | | Unmethylated in skeletal |
| | | | | | | | | muscle |
| 56 | 272 | 273 | 594 | 595 | | | | Unmethylated in |
| | | | | | | | | Keratinocyctes |
| 57 | 274 | 275 | 596 | 597 | C20orf102 | | ENSG00000132821 | Unmethylated in |
| | | | | | | | | Keratinocyctes |
| 58 | 276 | 277 | 598 | 599 | | | | Unmethylated in fibroblasts |
| 59 | 278 | 279 | 600 | 601 | | | | Methylated in Keratinocyctes |
| 60 | 280 | 281 | 602 | 603 | | | | Methylated in CD4/CD8 |
| 61 | 282 | 283 | 604 | 605 | | | | Unmethylated in |
| | | | | | | | | Keratinocyctes |
| 62 | 284 | 285 | 606 | 607 | | | | Unmethylated in skeletal |
| | | | | | | | | muscle |
| 63 | 286 | 287 | 608 | 609 | | | | Unmethylated in Melanocytes |
| 64 | 288 | 289 | 610 | 611 | | | | Unmethylated in fibroblasts |
| 65 | 290 | 291 | 612 | 613 | | | | Unmethylated in skeletal |
| | | | | | | | | muscle |
| 66 | 292 | 293 | 614 | 615 | | | | Unmethylated in fibroblasts |
| 67 | 294 | 295 | 616 | 617 | | | | Unmethylated in Melanocytes |
| 68 | 296 | 297 | 618 | 619 | | | | Unmethylated in fibroblasts |
| 69 | 298 | 299 | 620 | 621 | | | | Unmethylated in fibroblasts |
| 70 | 300 | 301 | 622 | 623 | | | | Unmethylated in Melanocytes |
| 71 | 302 | 303 | 624 | 625 | SULF2 | Extracellular sulfatase | ENSG00000196562 | Unmethylated in fibroblasts |
| | | | | | | Sulf-2 precursor | | |
| 72 | 304 | 305 | 626 | 627 | RP11-290F20.1 | | OTTHUMG00000032719 | Unmethylated in fibroblasts |
| 73 | 306 | 307 | 628 | 629 | C20orf94 | chromosome 20 open | OTTHUMG00000031873 | Unmethylated in CD4 |
| | | | | | | reading frame 94 | | |
| 74 | 308 | 309 | 630 | 631 | C20orf82 | chromosome 20 open | OTTHUMG00000031902 | Unmethylated in fibroblasts |
| | | | | | | reading frame 82 | | |
| 75 | 310 | 311 | 632 | 633 | PCSK2 | proprotein convertase | OTTHUMG00000031941 | Unmethylated in fibroblasts |
| | | | | | | subtilisin/kexin type 2 | | |
| 76 | 312 | 313 | 634 | 635 | PCSK2 | proprotein convertase | OTTHUMT00000078120 | Unmethylated in Melanocytes |
| | | | | | | subtilisin/kexin type 2 | | |
| 77 | 314 | 315 | 636 | 637 | SNX5 | sorting nexin 5 | OTTHUMG00000031953 | Methylated in fibroblasts |
| 78 | 316 | 317 | 638 | 639 | SLC24A3 | solute carrier family 24 | OTTHUMG00000031993 | Unmethylated in skeletal |
| | | | | | | (sodium/potassium/calcium | | muscle |
| | | | | | | exchanger), member 3 | | |
| 79 | 318 | 319 | 640 | 641 | SLC24A3 | solute carrier family 24 | OTTHUMG00000031993 | Unmethylated in skeletal |
| | | | | | | (sodium/potassium/calcium | | muscle |
| | | | | | | exchanger), member 3 | | |
| 80 | 320 | 321 | 642 | 643 | CT026_HUMAN | | ENSG00000089101 | Unmethylated in fibroblasts |
| 81 | 322 | 323 | 644 | 645 | CT026_HUMAN | | ENSG00000089101 | Unmethylated in fibroblasts |
| 82 | 324 | 325 | 646 | 647 | Q9ULE8_HUMAN | | ENSG00000188559 | Unmethylated in |
| | | | | | | | | Keratinocyctes |
| 83 | 326 | 327 | 648 | 649 | Q9ULE8_HUMAN | | ENSG00000188559 | Unmethylated in liver |
| 84 | 328 | 329 | 650 | 651 | Q9ULE8_HUMAN | | ENSG00000188559 | Unmethylated in liver |
| 85 | 330 | 331 | 652 | 653 | Q9ULE8_HUMAN | | ENSG00000188559 | Unmethylated in |
| | | | | | | | | Keratinocyctes |
| 86 | 332 | 333 | 654 | 655 | Q9ULE8_HUMAN | | ENSG00000188559 | Unmethylated in |
| | | | | | | | | Keratinocyctes |
| 87 | 334 | 335 | 656 | 657 | PLAGL2 | Zinc finger protein | ENSG00000126003 | Unmethylated in skeletal |
| | | | | | | PLAGL2 (Pleiomorphic | | muscle |
| | | | | | | adenoma-like protein 2 | | |
| 88 | 336 | 337 | 658 | 659 | CT112_HUMAN | | ENSG00000197183 | Unmethylated in Melanocytes |
| 89 | 338 | 339 | 660 | 661 | PTPRT | protein tyrosine | OTTHUMG00000033040 | Unmethylated in Melanocytes |
| | | | | | | phosphatase, receptor type, T | | |
| 90 | 340 | 341 | 662 | 663 | SDC4 | Syndecan 4 | ENSG00000124145 | Methylated in CD4/CD8 |
| 91 | 342 | 343 | 664 | 665 | CDH22 | cadherin like 22 | OTTHUMG00000033073 | Methylated in Keratinocyctes |
| 92 | 344 | 345 | 666 | 667 | EYA2 | Eyes absent homolog 2 | ENSG00000064655 | Unmethylated in skeletal |
| | | | | | | | | muscle |
| 93 | 346 | 347 | 668 | 669 | SULF2 | Sulfatase2 | ENSG00000196562 | Unmethylated in CD4/CD8 |
| 94 | 348 | 349 | 670 | 671 | KCNB1 | potassium voltage-gated | OTTHUMG00000033051 | Methylated in liver |
| | | | | | | channel, Shab-related | | |
| | | | | | | subfamily, member 1 | | |
| 95 | 350 | 351 | 672 | 673 | BCAS4 | Breast carcinoma amplified | ENSG00000124243 | Methylated in melanocytes |
| | | | | | | sequence 4 | | |
| 96 | 352 | 353 | 674 | 675 | NFATC2 | nuclear factor of activated | OTTHUMG00000032747 | Unmethylated in CD4/CD8 |
| | | | | | | T-cells, | | |
| 97 | 354 | 355 | 676 | 677 | NFATC2 | nuclear factor of activated | OTTHUMG00000032747 | Unmethylated in CD4/CD8 |
| | | | | | | T-cells, | | |
| 98 | 356 | 357 | 678 | 679 | NP_775915.1 | | ENSG00000176659 | Unmethylated in skeletal |
| | | | | | | | | Muscle |
| 99 | 358 | 359 | 680 | 681 | BMP7 | bone morphogenetic | OTTHUMG00000032812 | Methylated in liver |
| | | | | | | protein 7 | | |

| | | | | | | | |
|---|---|---|---|---|---|---|---|
| 844 | 1256 | 1257 | 2080 | 2081 | FLOT1, flotillin 1, ENSG00000137312 | ENSG00000137312 | See tables 3 & 4 |
| 845 | 1258 | 1259 | 2082 | 2083 | C6orf25, chromosome 6 open reading frame | ENSG00000096148 | See tables 3 & 4 |
| | | | | | 25, ENSG00000096148 | | |
| 846 | 1260 | 1261 | 2084 | 2085 | VARS, valyl-tRNA synthetase, | ENSG00000096171 | See tables 3 & 4 |
| | | | | | ENSG00000096171 | | |
| 847 | 1262 | 1263 | 2086 | 2087 | major histocompatibility complex, class II, | OTTHUMG00000031076 | See tables 3 & 4 |
| | | | | | DP beta 1, OTTHUMG00000031076, HLA- | | |
| | | | | | DPB1 | | |
| 848 | 1264 | 1265 | 2088 | 2089 | HLA-DRB5, major histocompatibility | OTTHUMG00000031027 | See tables 3 & 4 |
| | | | | | complex, class II, DR beta 5, | | |
| | | | | | OTTHUMG00000031027 | | |
| 849 | 1266 | 1267 | 2090 | 2091 | COL11A2, collagen, type XI, alpha 2, | OTTHUMG00000031036 | See tables 3 & 4 |
| | | | | | OTTHUMG00000031036 | | |
| 850 | 1268 | 1269 | 2092 | 2093 | PRAME, Melanoma antigen preferentially | ENSG00000185686 | See tables 3 & 4 |
| | | | | | expressed in tumors (Preferentially expressed | | |
| | | | | | antigen of melanoma) (OPA-interacting | | |
| | | | | | protein 4) (OIP4), ENSG00000185686 | | |
| 851 | 1270 | 1271 | 2094 | 2095 | ZNRF3 protein (Fragment), | ENSG00000183579 | See tables 3 & 4 |
| | | | | | ENSG00000183579, ZNRF3 zinc and ring | | |
| | | | | | finger 3 (ZNRF3) | | |
| 852 | 1272 | 1273 | 2096 | 2097 | AP000357.2 (Vega gene ID), Pseudogene | OTTHUMG00000030571 | See tables 3 & 4 |
| 853 | 1274 | 1275 | 2098 | 2099 | AP000357.3 (Vega gene ID), Pseudogene | OTTHUMG00000030574 | See tables 3 & 4 |
| 854 | 1276 | 1277 | 2100 | 2101 | solute carrier family 7 (cationic amino acid | OTTHUMG00000030129 | See tables 3 & 4 |
| | | | | | transporter, y+ system), member 4, | | |
| | | | | | OTTHUMG00000030129, | | |
| 855 | 1278 | 1279 | 2102 | 2103 | Myosin-18B (Myosin XVIIIb), | ENSG00000133454 | See tables 3 & 4 |
| | | | | | ENSG00000133454, MYO18B | | |
| 856 | 1280 | 1281 | 2104 | 2105 | Q6ICL0_HUMAN (Predicted | ENSG00000184004 | See tables 3 & 4 |
| | | | | | UniProt/TrEMBL ID), hypothetical protein | | |
| | | | | | FLJ3257; ENSG00000184004 | | |
| 857 | 1282 | 1283 | 2106 | 2107 | FBLN1; fibulin 1; ENSG00000077942 | ENSG00000077942 | See tables 3 & 4 |
| 858 | 1284 | 1285 | 2108 | 2109 | CYP2D6; cytochrome P450, family 2, | ENSG00000100197 | See tables 3 & 4 |
| | | | | | subfamily D, polypeptide 6; | | |
| | | | | | ENSG00000100197 | | |
| 859 | 1286 | 1287 | 2110 | 2111 | AC008132.9 (Vega gene ID); Pseudogene; | OTTHUMG00000030688 | See tables 3 & 4 |
| | | | | | OTTHUMG00000030688 | | |
| 860 | 1288 | 1289 | 2112 | 2113 | glycoprotein Ib (platelet), beta polypeptide, | OTTHUMT00000075045 | See tables 3 & 4 |
| 861 | 1290 | 1291 | 2114 | 2115 | no gene associated | | See tables 3 & 4 |
| 862 | 1292 | 1293 | 2116 | 2117 | AC006548.8 (Vega gene ID) | OTTHUMG00000030274 | See tables 3 & 4 |
| 863 | 1294 | 1295 | 2118 | 2119 | OTTHUMG00000030650, AC005399.2, | OTTHUMG00000030650 | See tables 3 & 4 |
| | | | | | putativer processed transcribed | | |
| 864 | 1296 | 1297 | 2120 | 2121 | topoisomerase (DNA) III beta, | OTTHUMG00000030764 | See tables 3 & 4 |
| | | | | | OTTHUMG00000030764, TOP3B ( | | |
| 865 | 1298 | 1299 | 2122 | 2123 | no gene associated | | See tables 3 & 4 |
| 866 | 1300 | 1301 | 2124 | 2125 | KB-1269D1.3 (Vega gene ID); Pseudogene; | OTTHUMG00000030694 | See tables 3 & 4 |
| 867 | 1302 | 1303 | 2126 | 2127 | GPR24; G protein-coupled receptor 24; | ENSG00000128285 | See tables 3 & 4 |
| | | | | | ENSG00000128285 | | |
| 868 | 1304 | 1305 | 2128 | 2129 | GAL3ST1; galactose-3-O-sulfotransferase 1; | ENSG00000128242 | See tables 3 & 4 |
| | | | | | ENSG00000128242 | | |
| 869 | 1306 | 1307 | 2130 | 2131 | Cat eye syndrome critical region protein 5 | ENSG00000069998 | See tables 3 & 4 |
| | | | | | precursor, | | |
| 870 | 1308 | 1309 | 2132 | 2133 | HORMAD2; HORMA domain containing 2; | ENSG00000176635 | See tables 3 & 4 |
| | | | | | ENSG00000176635 | | |
| 871 | 1310 | 1311 | 2134 | 2135 | OTTHUMG00000030922, RP3-438O4.2 | OTTHUMG00000030922 | See tables 3 & 4 |
| 872 | 1312 | 1313 | 2136 | 2137 | NP_997357.1 (RefSeq peptide ID); | ENSG00000169668 | See tables 3 & 4 |
| | | | | | ENSG00000169668 | | |
| 873 | 1314 | 1315 | 2138 | 2139 | OTTHUMG00000030574, AP000357.3, | OTTHUMG00000030574 | See tables 3 & 4 |
| | | | | | novel pseudogene | | |
| 874 | 1316 | 1317 | 2140 | 2141 | LA16c-4G1.2 (Vega gene ID); Pseudogene; | OTTHUMG00000030832 | See tables 3 & 4 |
| | | | | | OTTHUMG00000030832 | | |
| 875 | 1318 | 1319 | 2142 | 2143 | KB-226F1.11 (Vega gene ID), embryonic | OTTHUMG00000030123 | See tables 3 & 4 |
| | | | | | marker, OTTHUMG00000030123 | | |
| 876 | 1320 | 1321 | 2144 | 2145 | OTTHUMG00000030780, CTA-373H7.4, | OTTHUMG00000030780 | See tables 3 & 4 |
| | | | | | novel pseudogene | | |
| 877 | 1322 | 1323 | 2146 | 2147 | RP1-47A17.8 (Vega gene ID); | OTTHUMG00000030878 | See tables 3 & 4 |
| | | | | | OTTHUMG00000030878 | | |
| 878 | 1324 | 1325 | 2148 | 2149 | RP4-539M6.7 (Vega gene ID); Pseudogene; | OTTHUMG00000030918 | See tables 3 & 4 |
| | | | | | OTTHUMG00000030918 | | |
| 879 | 1326 | 1327 | 2150 | 2151 | CSDC2; cold shock domain containing C2, | ENSG00000172346 | See tables 3 & 4 |
| | | | | | RNA binding; ENSG00000172346 | | |
| 880 | 1328 | 1329 | 2152 | 2153 | Gamma-parvin, PARVG | ENSG00000138964 | See tables 3 & 4 |
| 881 | 1330 | 1331 | 2154 | 2155 | OTTHUMG00000030167, CTA-243E7.3 | OTTHUMG00000030167 | See tables 3 & 4 |
| 882 | 1332 | 1333 | 2156 | 2157 | Oncostatin M precursor (OSM), | ENSG00000099985 | See tables 3 & 4 |
| | | | | | ENSG00000099985, OSM | | |
| 883 | 1334 | 1335 | 2158 | 2159 | Oncostatin M precursor (OSM), | ENSG00000099985 | See tables 3 & 4 |
| | | | | | ENSG00000099985, OSM | | |
| 884 | 1336 | 1337 | 2160 | 2161 | Myosin-18B (Myosin XVIIIb), MYO18B | ENSG00000133454 | See tables 3 & 4 |
| 885 | 1338 | 1339 | 2162 | 2163 | Q6ICL0_HUMAN (Predicted | ENSG00000184004 | See tables 3 & 4 |
| | | | | | UniProt/TrEMBL ID), hypothetical protein | | |
| | | | | | FLJ3257; ENSG00000184004 | | |
| 886 | 1340 | 1341 | 2164 | 2165 | OTTHUMG00000030140, CTA-299D3.6 | OTTHUMG00000030140 | See tables 3 & 4 |
| 887 | 1342 | 1343 | 2166 | 2167 | GALR3; galanin receptor 3; | ENSG00000128310 | See tables 3 & 4 |
| | | | | | ENSG00000128310 | | |
| 888 | 1344 | 1345 | 2168 | 2169 | GALR3; galanin receptor 3; | ENSG00000128310 | See tables 3 & 4 |
| | | | | | ENSG00000128310 | | |
| 889 | 1346 | 1347 | 2170 | 2171 | IL2RB; interleukin 2 receptor, beta; | ENSG00000100385 | See tables 3 & 4 |
| | | | | | ENSG00000100385 | | |
| 890 | 1348 | 1349 | 2172 | 2173 | CTA-343C1.3 (Vega gene ID); Putative | OTTHUMG00000030151 | See tables 3 & 4 |
| | | | | | Processed transcript; | | |
| | | | | | OTTHUMG00000030151 | | |
| 891 | 1350 | 1351 | 2174 | 2175 | CTA-941F9.6 (Vega_gene ID) | OTTHUMG00000030231 | See tables 3 & 4 |
| 892 | 1352 | 1353 | 2176 | 2177 | CTA-941F9.6 (Vega_gene ID) | OTTHUMG00000030231 | See tables 3 & 4 |
| 893 | 1354 | 1355 | 2178 | 2179 | LL22NC03-121E8.1 (Vega gene ID); Novel | OTTHUMG00000030676 | See tables 3 & 4 |
| | | | | | Protein coding; OTTHUMG00000030676 | | |
| 894 | 1356 | 1357 | 2180 | 2181 | Cytohesin-4, ENSG00000100055, PSCD4 | ENSG00000100055 | See tables 3 & 4 |
| 895 | 1358 | 1359 | 2182 | 2183 | RP4-754E20_A.4 (Vega gene ID); Putative | OTTHUMG00000030716 | See tables 3 & 4 |
| | | | | | Processed transcript; | | |
| | | | | | OTTHUMG00000030716 | | |
| 896 | 1360 | 1361 | 2184 | 2185 | PIB5PA; phosphatidylinositol (4,5) | ENSG00000185133 | See tables 3 & 4 |
| | | | | | bisphosphate 5-phosphatase, A; | | |
| | | | | | ENSG00000185133; embryonic marker | | |
| 897 | 1362 | 1363 | 2186 | 2187 | no gene associated | | See tables 3 & 4 |
| 898 | 1364 | 1365 | 2188 | 2189 | PLA2G3; ENSG00000100078; | ENSG00000100078 | See tables 3 & 4 |
| | | | | | phospholipase A2, group III | | |
| 899 | 1366 | 1367 | 2190 | 2191 | PLA2G3; ENSG00000100078; | ENSG00000100078 | See tables 3 & 4 |
| | | | | | phospholipase A2, group III | | |
| 900 | 1368 | 1369 | 2192 | 2193 | DGCR2; DiGeorge syndrome critical region | ENSG00000070413 | See tables 3 & 4 |
| | | | | | gene 2; ENSG00000070413 | | |
| 901 | 1370 | 1371 | 2194 | 2195 | TCN2; transcobalamin II; macrocytic | ENSG00000185339 | See tables 3 & 4 |
| | | | | | anemia; ENSG00000185339 | | |
| 902 | 1372 | 1373 | 2196 | 2197 | IGLL1; immunoglobulin lambda-like | ENSG00000128322 | See tables 3 & 4 |
| | | | | | polypeptide 1; ENSG00000128322 | | |
| 903 | 1374 | 1375 | 2198 | 2199 | RP1-29C18.7 (Vega gene ID); Novel | OTTHUMG00000030424 | See tables 3 & 4 |
| | | | | | Processed transcript; | | |
| | | | | | OTTHUMG00000030424 | | |
| 904 | 1376 | 1377 | 2200 | 2201 | IGLC1; immunoglobulin lambda constant 1 | ENSG00000100208 | See tables 3 & 4 |
| | | | | | (Mcg marker); ENSG00000100208 | | |
| 905 | 1378 | 1379 | 2202 | 2203 | APOBEC3B; apolipoprotein B mRNA | ENSG00000179750 | See tables 3 & 4 |
| | | | | | editing enzyme, catalytic polypeptide-like | | |
| | | | | | 3B; ENSG00000179750 | | |
| 906 | 1380 | 1381 | 2204 | 2205 | CRYBB1; crystallin, beta B1; | ENSG00000100122 | See tables 3 & 4 |
| | | | | | ENSG00000100122 | | |
| 907 | 1382 | 1383 | 2206 | 2207 | CRYBA4; crystallin, beta A4; | ENSG00000196431 | See tables 3 & 4 |
| | | | | | ENSG00000196431 | | |
| 908 | 1384 | 1385 | 2208 | 2209 | sushi domain containing 2, SUSD2 | ENSG00000099994 | See tables 3 & 4 |
| 909 | 1386 | 1387 | 2210 | 2211 | sushi domain containing 2, SUSD2 | ENSG00000099994 | See tables 3 & 4 |
| 910 | 1388 | 1389 | 2212 | 2213 | OTTHUMG00000030870, Putative Processed | OTTHUMG00000030870 | See tables 3 & 4 |
| | | | | | transcript, CTA-503F6.1 | | |
| 911 | 1390 | 1391 | 2214 | 2215 | OTTHUMG00000030800, KB-1323B2.3 | OTTHUMG00000030800 | See tables 3 & 4 |
| 912 | 1392 | 1393 | 2216 | 2217 | no gene associated | | See tables 3 & 4 |
| 913 | 1394 | 1395 | 2218 | 2219 | IGLV1-44; immunoglobulin lambda variable | ENSG00000186751 | See tables 3 & 4 |
| | | | | | 1-44; ENSG00000186751 | | |
| 914 | 1396 | 1397 | 2220 | 2221 | IGLV1-44; immunoglobulin lambda variable | ENSG00000186751 | See tables 3 & 4 |
| | | | | | 1-44; ENSG00000186751 | | |
| 915 | 1398 | 1399 | 2222 | 2223 | OTTHUMG00000030922, RP3-438O4.2 | OTTHUMG00000030922 | See tables 3 & 4 |
| 916 | 1400 | 1401 | 2224 | 2225 | OTTHUMG00000030922, RP3-438O4.2 | OTTHUMG00000030922 | See tables 3 & 4 |
| 917 | 1402 | 1403 | 2226 | 2227 | APOL4; apolipoprotein L, 4; | ENSG00000100336 | See tables 3 & 4 |
| | | | | | ENSG00000100336 | | |
| 918 | 1404 | 1405 | 2228 | 2229 | OTTHUMG00000030852, RP4- | OTTHUMG00000030852 | See tables 3 & 4 |
| | | | | | 756G23.1, novel processed transcript | | |
| 919 | 1406 | 1407 | 2230 | 2231 | ENSG00000100399, | ENSG00000100399 | See tables 3 & 4 |
| 920 | 1408 | 1409 | 2232 | 2233 | Neutrophil cytosol factor 4 (NCF-4) | ENSG00000100365 | See tables 3 & 4 |
| | | | | | (Neutrophil NADPH oxidase factor 4) (p40- | | |
| | | | | | phox) (p40phox)., ENSG00000100365, | | |
| | | | | | NCF4 | | |
| 921 | 1410 | 1411 | 2234 | 2235 | Neutrophil cytosol factor 4 (NCF-4) | ENSG00000100365 | See tables 3 & 4 |
| | | | | | (Neutrophil NADPH oxidase factor 4) (p40- | | |
| | | | | | phox) (p40phox)., ENSG00000100365, | | |
| | | | | | NCF4 | | |
| 922 | 1412 | 1413 | 2236 | 2237 | Somatostatin receptor type 3 (SS3R) (SSR- | ENSG00000183473 | See tables 3 & 4 |
| | | | | | 28), D | | |
| 923 | 1414 | 1415 | 2238 | 2239 | Somatostatin receptor type 3 (SS3R) (SSR- | ENSG00000183473 | See tables 3 &4 |
| | | | | | 28), D; SSTR3 | | |
| 924 | 1416 | 1417 | 2240 | 2241 | Bcl-2 interacting killer (Apoptosis inducer | ENSG00000100290 | See tables 3 & 4 |
| | | | | | NBK) (BP4) (BIP1)., BIK | | |
| 925 | 1418 | 1419 | 2242 | 2243 | GAS2-like protein 1 (Growth arrest-specific | ENSG00000185340 | See tables 3 & 4 |
| | | | | | 2-like 1) (GAS2-related protein on | | |
| | | | | | chromosome 22) (GAR22 protein), GAS2L1 | | |
| 926 | 1420 | 1421 | 2244 | 2245 | RP3-355C18.2 (Vega gene ID) | OTTHUMG00000030072 | See tables 3 & 4 |
| 927 | 1422 | 1423 | 2246 | 2247 | SOX10; SRY (sex determining region Y)- | ENSG00000100146 | See tables 3 & 4 |
| | | | | | box 10; ENSG00000100146 | | |
| 928 | 1424 | 1425 | 2248 | 2249 | Gamma-parvin ENSG00000138964 | ENSG00000138964 | See tables 3 & 4 |
| 929 | 1426 | 1427 | 2250 | 2251 | Caspase recruitment domain protein 10 | ENSG00000100065 | See tables 3 & 4 |
| | | | | | (CARD-containing MAGUK protein 3) | | |
| | | | | | (Carma 3). ENSG00000100065, CARD10 | | |
| 930 | 1428 | 1429 | 2252 | 2253 | ENSG00000100101, NP_077289.1 | ENSG00000100101 | See tables 3 & 4 |
| 931 | 1430 | 1431 | 2254 | 2255 | HTF9C; HpaII tiny fragments locus 9C; | ENSG00000099899 | See tables 3 & 4 |
| | | | | | ENSG00000099899 | | |
| 932 | 1432 | 1433 | 2256 | 2257 | Oncostatin M precursor (OSM), | ENSG00000099985 | See tables 3 & 4 |
| | | | | | ENSG00000099985, OSM | | |
| 933 | 1434 | 1435 | 2258 | 2259 | CTA-407F11.4 (Vega gene ID); Novel | OTTHUMG00000030804 | See tables 3 & 4 |
| | | | | | Processed transcript; | | |
| | | | | | OTTHUMG00000030804 | | |
| 934 | 1436 | 1437 | 2260 | 2261 | Q6ICL0_HUMAN (Predicted | ENSG00000184004 | See tables 3 & 4 |
| | | | | | UniProt/TrEMBL ID), hypothetical protein | | |
| | | | | | FLJ3257; ENSG00000184004 | | |
| 935 | 1438 | 1439 | 2262 | 2263 | CTA-989H11.2 (Vega gene ID); Putative | OTTHUMG00000030141 | See tables 3 & 4 |
| | | | | | Processed transcript; | | |
| | | | | | OTTHUMG00000030141 | | |
| 936 | 1440 | 1441 | 2264 | 2265 | transmembrane protease, serine 6 | ENSG00000187045 | See tables 3 & 4 |
| 937 | 1442 | 1443 | 2266 | 2267 | HMG2L1; high-mobility group protein 2-like | ENSG00000100281 | See tables 3 & 4 |
| | | | | | 1; ENSG00000100281 | | |
| 938 | 1444 | 1445 | 2268 | 2269 | NP_001017964.1 (RefSeq peptide ID); | ENSG00000161179 | See tables 3 & 4 |
| | | | | | hypothetical protein LOC150223; | | |
| | | | | | ENSG00000161179 | | |
| 939 | 1446 | 1447 | 2270 | 2271 | Platelet-derived growth factor B chain | ENSG00000100311 | See tables 3 & 4 |
| | | | | | precursor (PDGF B-chain, | | |
| 940 | 1448 | 1449 | 2272 | 2273 | OTTHUMG00000030815, | OTTHUMG00000030815 | See tables 3 & 4 |
| 941 | 1450 | 1451 | 2274 | 2275 | MGAT3; mannosyl (beta-1,4-)-glycoprotein | ENSG00000128268 | See tables 3 & 4 |
| | | | | | beta-1,4-N-acetylglucosaminyltransferase; | | |
| | | | | | ENSG00000128268 | | |
| 942 | 1452 | 1453 | 2276 | 2277 | Ceramide kinase (EC 2.7.1.138) | ENSG00000100422 | See tables 3 & 4 |
| | | | | | (Acylsphingosine kinase) (hCERK) (Lipid | | |
| | | | | | kinase 4) (LK4), ENSG00000100422, | | |
| | | | | | CERK | | |
| 943 | 1454 | 1455 | 2278 | 2279 | Reticulon 4 receptor precursor (Nogo | ENSG00000040608 | See tables 3 & 4 |
| | | | | | receptor) (NgR) (Nogo-66 receptor), RTN4R | | |
| 944 | 1456 | 1457 | 2280 | 2281 | UNC84B; unc-84 homolog B (C. Elegans); | ENSG00000100242 | See tables 3 & 4 |
| | | | | | ENSG00000100242 | | |
| 945 | 1458 | 1459 | 2282 | 2283 | RABL4; RAB, member of RAS oncogene | ENSG00000100360 | See tables 3 & 4 |
| | | | | | family-like 4; ENSG00000100360 | | |
| 946 | 1460 | 1461 | 2284 | 2285 | Cadherin EGF LAG seven-pass G-type | ENSG00000075275 | See tables 3 & 4 |
| | | | | | receptor 1 precursor (Flamingo homolog 2) | | |
| | | | | | (hFmi2), CELSR1 | | |
| 947 | 1462 | 1463 | 2286 | 2287 | OTTHUMG00000030326, LL22NC03- | OTTHUMG00000030326 | See tables 3 & 4 |
| | | | | | 5H6.1 | | |
| 948 | 1464 | 1465 | 2288 | 2289 | OTTHUMG00000030656, RP3-515N1.6 | OTTHUMG00000030656 | See tables 3 & 4 |
| 949 | 1466 | 1467 | 2290 | 2291 | SMTN; smoothelin; ENSG00000183963 | ENSG00000183963 | See tables 3 & 4 |
| 950 | 1468 | 1469 | 2292 | 2293 | ZNRF3 protein (Fragment), | ENSG00000183579 | See tables 3 & 4 |
| | | | | | ENSG00000183579, ZNRF3 zinc and ring | | |
| | | | | | finger 3 (ZNRF3) | | |
| 951 | 1470 | 1471 | 2294 | 2295 | OTTHUMG00000030700, GRB2-related | OTTHUMG00000030700 | See tables 3 & 4 |
| | | | | | adaptor protein 2, GRAP2 | | |
| 952 | 1472 | 1473 | 2296 | 2297 | CAP-binding protein complex interacting | ENSG00000186976 | See tables 3 & 4 |
| | | | | | protein 1 isoform a Source: RefSeq_peptide | | |
| | | | | | NP_073622 | | |
| 953 | 1474 | 1475 | 2298 | 2299 | SAM50_HUMAN (UniProt/Swiss-Prot ID), | ENSG00000100347 | See tables 3 & 4 |
| | | | | | ENSG00000100347, SAM50-like protein | | |
| | | | | | CGI-51; sorting and assembly machinery | | |
| | | | | | component 50 homolog (S. Cerevisiae) | | |
| 954 | 1476 | 1477 | 2300 | 2301 | SULT4A1; sulfotransferase family 4A, | ENSG00000130540 | See tables 3 & 4 |
| | | | | | member 1; ENSG00000130540 | | |
| 955 | 1478 | 1479 | 2302 | 2303 | TIMP3; TIMP metallopeptidase inhibitor 3 | ENSG00000100234 | See tables 3 & 4 |
| | | | | | (Sorsby fundus dystrophy, | | |
| | | | | | pseudoinflammatory); ENSG00000100234 | | |
| 956 | 1480 | 1481 | 2304 | 2305 | T-box transcription factor TBX1 (T-box | ENSG00000184058 | See tables 3 & 4 |
| | | | | | protein 1) (Testis-specific T-box protein), | | |
| 957 | 1482 | 1483 | 2306 | 2307 | MPPED1, metallophosphoesterase domain | ENSG00000186732 | See tables 3 & 4 |
| | | | | | containing 1 | | |
| 958 | 1484 | 1485 | 2308 | 2309 | ENSG00000188511 NP_942148.1 novel | ENSG00000188511 | See tables 3 & 4 |
| | | | | | Gene hypothetical protein LOC348645 | | |
| 959 | 1486 | 1487 | 2310 | 2311 | Cdc42 effector protein 1, | ENSG00000128283 | See tables 3 & 4 |
| 960 | 1488 | 1489 | 2312 | 2313 | RPL3; ribosomal protein L3; | ENSG00000100316 | See tables 3 & 4 |
| | | | | | ENSG00000100316 | | |
| 961 | 1490 | 1491 | 2314 | 2315 | APOL2; apolipoprotein L, 2; | ENSG00000128335 | See tables 3 & 4 |
| | | | | | ENSG00000128335 | | |
| 962 | 1492 | 1493 | 2316 | 2317 | RAC2; ras-related C3 botulinum toxin | ENSG00000128340 | See tables 3 & 4 |
| | | | | | substrate 2 (rho family, small GTP binding | | |
| | | | | | protein Rac2); ENSG00000128340 | | |
| 963 | 1494 | 1495 | 2318 | 2319 | OTTHUMP00000028917, Q96E60 | ENSG00000100399 | See tables 3 & 4 |
| 964 | 1496 | 1497 | 2320 | 2321 | Neutrophil cytosol factor 4 (NCF-4) | ENSG00000100365 | See tables 3 & 4 |
| | | | | | (Neutrophil NADPH oxidase factor 4) (p40- | | |
| | | | | | phox) (p40phox)., ENSG00000100365, | | |
| | | | | | NCF4 | | |
| 965 | 1498 | 1499 | 2322 | 2323 | XP_371837.1 (RefSeq peptide predicted ID); | ENSG00000168768 | See tables 3 & 4 |
| | | | | | PREDICTED: similar to oxidoreductase | | |
| | | | | | UCPA Source: RefSeq_peptide_predicted | | |
| | | | | | XP_371837; ENSG00000168768 | | |
| 966 | 1500 | 1501 | 2324 | 2325 | triggering receptor expressed on myeloid | ENSG00000112195 | See tables 3 & 4 |
| | | | | | cells-like 2, ENSG00000112195, TREML2 | | |
| 967 | 1502 | 1503 | 2326 | 2327 | TREML1; triggering receptor expressed on | ENSG00000161911 | See tables 3 & 4 |
| | | | | | myeloid cells-like 1; ENSG00000161911 | | |
| 968 | 1504 | 1505 | 2328 | 2329 | ENSG00000178199, Q6ZRW2_HUMAN; | ENSG00000178199 | See tables 3 & 4 |
| | | | | | zinc finger CCCH-type containing 12D | | |
| 969 | 1506 | 1507 | 2330 | 2331 | AIM1; absent in melanoma1; | ENSG00000112297 | See tables 3 & 4 |
| | | | | | ENSG00000112297 | | |
| 970 | 1508 | 1509 | 2332 | 2333 | NKG2D ligand 4 precursor (NKG2D ligand | ENSG00000164520 | See tables 3 & 4 |
| | | | | | 4) (NKG2DL4) (N2DL-4) (Retinoic acid | | |
| | | | | | early transcript 1E) (Lymphocyte effector | | |
| | | | | | toxicity activation ligand) (RAE-1-like | | |
| | | | | | transcript 4) (RL-4), | | |
| 971 | 1510 | 1511 | 2334 | 2335 | Disheveled associated activator of | ENSG00000146122 | See tables 3 & 4 |
| | | | | | morphogenesis 2, ENSG00000146122, | | |
| | | | | | DAAM2 | | |
| 972 | 1512 | 1513 | 2336 | 2337 | RP11-535K1.1 (Vega gene ID); Putative | OTTHUMG00000014660 | See tables 3 & 4 |
| | | | | | Processed transcript; | | |
| | | | | | OTTHUMG00000014660 | | |
| 973 | 1514 | 1515 | 2338 | 2339 | OTTHUMG00000015679; Novel Protein | OTTHUMG00000015679 | See tables 3 & 4 |
| | | | | | coding; RP3-509I19.3 | | |
| 974 | 1516 | 1517 | 2340 | 2341 | RP11-503C24.1 (Vega gene ID); Putative | OTTHUMG00000016040 | See tables 3 & 4 |
| | | | | | Processed transcript; | | |
| | | | | | OTTHUMG00000016040 | | |
| 975 | 1518 | 1519 | 2342 | 2343 | GABRR2; gamma-aminobutyric acid | ENSG00000111886 | See tables 3 & 4 |
| | | | | | (GABA) receptor, rho 2; ENSG00000111886 | | |
| 976 | 1520 | 1521 | 2344 | 2345 | ANKRD6; ankyrin repeat domain 6; | ENSG00000135299 | See tables 3 & 4 |
| | | | | | ENSG00000135299 | | |
| 977 | 1522 | 1523 | 2346 | 2347 | TXLNB; taxilin beta; ENSG00000164440 | ENSG00000164440 | See tables 3 & 4 |
| 978 | 1524 | 1525 | 2348 | 2349 | TXLNB; taxilin beta; ENSG00000164440 | ENSG00000164440 | See tables 3 & 4 |
| 979 | 1526 | 1527 | 2350 | 2351 | RP5-899B16.2 (Vega gene ID); Putative | OTTHUMG00000015698 | See tables 3 & 4 |
| | | | | | Processed transcript; | | |
| | | | | | OTTHUMG00000015698 | | |
| 980 | 1528 | 1529 | 2352 | 2353 | Probable G-protein coupled receptor 116 | ENSG00000069122 | See tables 3 & 4 |
| | | | | | precursor, | | |
| 981 | 1530 | 1531 | 2354 | 2355 | RP11-146I2.1 (Vega gene ID); Novel | OTTHUMG00000014290 | See tables 3 & 4 |
| | | | | | Processed transcript; | | |
| | | | | | OTTHUMG00000014290 | | |
| 982 | 1532 | 1533 | 2356 | 2357 | GPR115; G protein-coupled receptor 115; | ENSG00000153294 | See tables 3 & 4 |
| | | | | | ENSG00000153294 | | |
| 983 | 1534 | 1535 | 2358 | 2359 | GPR126; G protein-coupled receptor 126; | ENSG00000112414 | See tables 3 & 4 |
| | | | | | ENSG00000112414 embryonic marker | | |
| 984 | 1536 | 1537 | 2360 | 2361 | RP1-60O19.1 (Vega gene ID); Known | OTTHUMG00000015305 | See tables 3 & 4 |
| | | | | | Processed transcript; | | |
| | | | | | OTTHUMG00000015305 | | |
| 985 | 1538 | 1539 | 2362 | 2363 | new gene!!!, OTTHUMG00000015313, | OTTHUMG00000015313 | See tables 3 & 4 |
| | | | | | RP1-47M23.1 SCML4 sex comb on midleg- | | |
| | | | | | like 4 (Drosophila) [Homo sapiens] | | |
| 986 | 1540 | 1541 | 2364 | 2365 | OTTHUMG00006004170 , TPX1testis | OTTHUMG00000014822 | See tables 3 & 4 |
| | | | | | specific protein 1 (probe H4-1 p3-1) | | |
| 987 | 1542 | 1543 | 2366 | 2367 | OTTHUMG00000014829, | OTTHUMG00000014829 | See tables 3 & 4 |
| 988 | 1544 | 1545 | 2368 | 2369 | OTTHUMG00000015337RP11-487F23.3 | OTTHUMG00000015337 | See tables 3 & 4 |
| | | | | | hypothetical LOC389422 | | |
| 989 | 1546 | 1547 | 2370 | 2371 | Nesprin-1 (Nuclear envelope spectrin repeat | ENSG00000131018 | See tables 3 & 4 |
| | | | | | protein 1) (Synaptic nuclear envelope protein | | |
| | | | | | 1) (Syne-1) (Myocyte nuclear envelope | | |
| | | | | | protein 1) (Myne-1) (Enaptin), | | |
| | | | | | ENSG00000131018, SYNE1 | | |
| 990 | 1548 | 1549 | 2372 | 2373 | Nesprin-1 (Nuclear envelope spectrin repeat | ENSG00000131018 | See tables 3 & 4 |
| | | | | | protein 1) (Synaptic nuclear envelope protein | | |
| | | | | | 1) (Syne-1) (Myocyte nuclear envelope | | |
| | | | | | protein 1) (Myne-1) (Enaptin), | | |
| | | | | | ENSG00000131018, SYNE1 | | |
| 991 | 1550 | 1551 | 2374 | 2375 | RP11-398K22.4 (Vega gene ID); Putative | OTTHUMG00000015024 | See tables 3 & 4 |
| | | | | | Processed transcript; | | |
| | | | | | OTTHUMG00000015024 | | |
| 992 | 1552 | 1553 | 2376 | 2377 | MyoD family inhibitor (Myogenic repressor | ENSG00000112559 | See tables 3 & 4 |
| | | | | | I-mf), MDFI | | |
| 993 | 1554 | 1555 | 2378 | 2379 | OTTHUMG00000014691, putative | OTTHUMG00000014691 | See tables 3 & 4 |
| | | | | | processed transcript, RP11-533O20.2 | | |
| 994 | 1556 | 1557 | 2380 | 2381 | RP3-398D13.4 (Vega gene ID); | OTTHUMG00000014188 | See tables 3 & 4 |
| | | | | | OTTHUMG00000014188 | | |
| 995 | 1558 | 1559 | 2382 | 2383 | RP3-429O6.1 (Vega gene ID); Putative | OTTHUMG00000014195 | See tables 3 & 4 |
| | | | | | Processed transcript; | | |
| | | | | | OTTHUMG00000014195 | | |
| 996 | 1560 | 1561 | 2384 | 2385 | MOG; myelin oligodendrocyte glycoprotein; | ENSG00000137345 | See tables 3 & 4 |
| | | | | | ENSG00000137345 | | |
| 997 | 1562 | 1563 | 2386 | 2387 | RP3-495K2.2 (Vega gene ID); Putative | OTTHUMG00000016052 | See tables 3 & 4 |
| | | | | | Processed transcript; | | |
| | | | | | OTTHUMG00000016052 | | |
| 998 | 1564 | 1565 | 2388 | 2389 | RP11-417E7.1 (Vega gene ID); Putative | OTTHUMG00000016054 | See tables 3 & 4 |
| | | | | | Processed transcript; | | |
| | | | | | OTTHUMG00000016054 | | |
| 999 | 1566 | 1567 | 2390 | 2391 | yrosine-protein kinase-like 7 precursor | ENSG00000112655 | See tables 3 & 4 |
| | | | | | (Colon carcinoma kinase 4) (CCK-4)., | | |
| | | | | | ENSG00000112655, PTK7 | | |
| 1000 | 1568 | 1569 | 2392 | 2393 | RP11-174C7.4 (Vega gene ID) | OTTHUMG00000015553 | See tables 3 & 4 |
| 1001 | 1570 | 1571 | 2394 | 2395 | cytidine monophosphate-N-acetylneuraminic | OTTHUMG00000016099 | See tables 3 & 4 |
| | | | | | acid hydroxylase (CMP-N-acetylneuraminate | | |
| | | | | | monooxygenase); CMAH | | |
| 1002 | 1572 | 1573 | 2396 | 2397 | PKHD1; polycystic kidney and hepatic | ENSG00000170927 | See tables 3 & 4 |
| | | | | | disease 1 (autosomal recessive); | | |
| | | | | | ENSG00000170927 | | |
| 1003 | 1574 | 1575 | 2398 | 2399 | RP3-471C18.2 (Vega gene ID); Novel | OTTHUMG00000014332 | See tables 3 & 4 |
| | | | | | Processed transcript; | | |
| | | | | | OTTHUMG00000014332 | | |
| 1004 | 1576 | 1577 | 2400 | 2401 | RP11-204E9.1 (Vega gene ID); Putative | OTTHUMG00000014342 | See tables 3 & 4 |
| | | | | | Processed transcript; | | |
| | | | | | OTTHUMG00000014342 | | |
| 1005 | 1578 | 1579 | 2402 | 2403 | glutathione peroxidase 5, | OTTHUMG00000016307 | See tables 3 & 4 |
| | | | | | OTTHUMG00000016307, GPX5 | | |
| 1006 | 1580 | 1581 | 2404 | 2405 | RP11-411K7.1 (Vega gene ID); Putative | OTTHUMG00000014887 | See tables 3 & 4 |
| | | | | | Processed transcript; | | |
| | | | | | OTTHUMG00000014887 | | |
| 1007 | 1582 | 1583 | 2406 | 2407 | skin marker, Glutamate receptor, ionotropic | ENSG00000164418 | See tables 3 & 4 |
| | | | | | kainate 2 precursor (Glutamate receptor 6) | | |
| | | | | | (GluR-6) (GluR6) (Excitatory amino acid | | |
| | | | | | receptor 4) (EAA4) | | |
| 1008 | 1584 | 1585 | 2408 | 2409 | C6orf142; chromosome 6 open reading frame | ENSG00000146147 | See tables 3 & 4 |
| | | | | | 142; ENSG00000146147 | | |
| 1009 | 1586 | 1587 | 2410 | 2411 | HDGFL1; hepatoma derived growth factor- | ENSG00000112273 | See tables 3 & 4 |
| | | | | | like 1; ENSG00000112273 | | |
| 1010 | 1588 | 1589 | 2412 | 2413 | forkhead box C1, OTTHUMG00000016182, | OTTHUMG00000016182 | See tables 3 & 4 |
| | | | | | FOXC1 | | |
| 1011 | 1590 | 1591 | 2414 | 2415 | C6orf188; chromosome 6 open reading frame | ENSG00000178033 | See tables 3 & 4 |
| | | | | | 188; ENSG00000178033 | | |
| 1012 | 1592 | 1593 | 2416 | 2417 | ME1; malic enzyme 1, NADP(+)-dependent, | ENSG00000065833 | See tables 3 & 4 |
| | | | | | cytosolic; ENSG00000065833 | | |
| 1013 | 1594 | 1595 | 2418 | 2419 | SLC22A1; solute carrier family 22 (organic | ENSG00000175003 | See tables 3 & 4 |
| | | | | | cation transporter), member 1 | | |
| 1014 | 1596 | 1597 | 2420 | 2421 | RP11-235G24.1 (Vega gene ID) | OTTHUMG00000015959 | See tables 3 & 4 |
| 1015 | 1598 | 1599 | 2422 | 2423 | T-box 18; TBX18 | ENSG00000112837 | See tables 3 & 4 |
| 1016 | 1600 | 1601 | 2424 | 2425 | CTA-31J9.2, putative processed transcript, | OTTHUMG00000015619 | See tables 3 & 4 |
| | | | | | OTTHUMG00000015619 | | |
| 1017 | 1602 | 1603 | 2426 | 2427 | RP1-32B1.4 (Vega gene ID); Putative | OTTHUMG00000015628 | See tables 3 & 4 |
| | | | | | Processed transcript | | |
| | | | | | OTTHUMG00000015628 | | |
| 1018 | 1604 | 1605 | 2428 | 2429 | OTTHUMG00000014223, RP11-203H2.2, | OTTHUMG00000014223 | See tables 3 & 4 |
| | | | | | novel processed treanscript | | |
| 1019 | 1606 | 1607 | 2430 | 2431 | OTTHUMG00000014737, C6orf154 and | OTTHUMG00000014737 | See tables 3 & 4 |
| | | | | | Name: chromosome 6 open reading frame | | |
| | | | | | 154; RP3-337H4.2 | | |
| 1020 | 1608 | 1609 | 2432 | 2433 | transcription factor AP-2 alpha, | OTTHUMG00000014235 | See tables 3 & 4 |
| | | | | | OTTHUMG00000014235, TFAP2A | | |
| 1021 | 1610 | 1611 | 2434 | 2435 | IL20RA; interleukin 20 receptor, alpha; | ENSG00000016402 | See tables 3 & 4 |
| | | | | | ENSG00000016402 | | |
| 1022 | 1612 | 1613 | 2436 | 2437 | KAAG1; kidney associated antigen 1; | ENSG00000146049 | See tables 3 & 4 |
| | | | | | ENSG00000146049 | | |
| 1023 | 1614 | 1615 | 2438 | 2439 | TGM3; transglutaminase 3 (E polypeptide, | ENSG00000125780 | See tables 3 & 4 |
| | | | | | protein-glutamine-gamma- | | |
| | | | | | glutamyltransferase); ENSG00000125780 | | |
| 1024 | 1616 | 1617 | 2440 | 2441 | RASSF2; Ras association (RalGDS/AF-6) | ENSG00000101265 | See tables 3 & 4 |
| | | | | | domain family 2; ENSG00000101265 | | |
| 1025 | 1618 | 1619 | 2442 | 2443 | no gene associated | | See tables 3 & 4 |
| 1026 | 1620 | 1621 | 2444 | 2445 | no gene associated | | See tables 3 & 4 |
| 1027 | 1622 | 1623 | 2446 | 2447 | no gene associated | | See tables 3 & 4 |
| 1028 | 1624 | 1625 | 2448 | 2449 | no gene associated | | See tables 3 & 4 |
| 1029 | 1626 | 1627 | 2450 | 2451 | no gene associated | | See tables 3 & 4 |
| 1030 | 1628 | 1629 | 2452 | 2453 | no gene associated | | See tables 3 & 4 |
| 1031 | 1630 | 1631 | 2454 | 2455 | no gene associated | | See tables 3 & 4 |
| 1032 | 1632 | 1633 | 2456 | 2457 | no gene associated | | See tables 3 & 4 |
| 1033 | 1634 | 1635 | 2458 | 2459 | no gene associated | | See tables 3 & 4 |
| 1034 | 1636 | 1637 | 2460 | 2461 | no gene associated | | See tables 3 & 4 |
| 1035 | 1638 | 1639 | 2462 | 2463 | no gene associated | | See tables 3 & 4 |
| 1036 | 1640 | 1641 | 2464 | 2465 | RP4-697P8.2 (Vega gene ID); Putative | OTTHUMG00000031879 | See tables 3 & 4 |
| | | | | | Processed transcript; | | |
| | | | | | OTTHUMG00000031879 | | |
| 1037 | 1642 | 1643 | 2466 | 2467 | no gene associated | | See tables 3 & 4 |
| 1038 | 1644 | 1645 | 2468 | 2469 | OTTHUMG00000031883, | OTTHUMG00000031883 | See tables 3 & 4 |
| 1039 | 1646 | 1647 | 2470 | 2471 | no gene associated | | See tables 3 & 4 |
| 1040 | 1648 | 1649 | 2472 | 2473 | no gene associated | | See tables 3 & 4 |
| 1041 | 1650 | 1651 | 2474 | 2475 | no gene associated | | See tables 3 & 4 |
| 1042 | 1652 | 1653 | 2476 | 2477 | no gene associated | | See tables 3 & 4 |
| 1043 | 1654 | 1655 | 2478 | 2479 | no gene associated | | See tables 3 & 4 |
| 1044 | 1656 | 1657 | 2480 | 2481 | Ras and Rab interactor 2, | OTTHUMG00000031996 | See tables 3 & 4 |
| 1045 | 1658 | 1659 | 2482 | 2483 | no gene associated | | See tables 3 & 4 |
| 1046 | 1660 | 1661 | 2484 | 2485 | no gene associated | | See tables 3 & 4 |
| 1047 | 1662 | 1663 | 2486 | 2487 | no gene associated | | See tables 3 & 4 |
| 1048 | 1664 | 1665 | 2488 | 2489 | no gene associated | | See tables 3 & 4 |
| 1049 | 1666 | 1667 | 2490 | 2491 | no gene associated | | See tables 3 & 4 |
| 1050 | 1668 | 1669 | 2492 | 2493 | no gene associated | | See tables 3 & 4 |
| 1051 | 1670 | 1671 | 2494 | 2495 | no gene associated | | See tables 3 & 4 |
| 1052 | 1672 | 1673 | 2496 | 2497 | no gene associated | | See tables 3 & 4 |
| 1053 | 1674 | 1675 | 2498 | 2499 | no gene associated | | See tables 3 & 4 |
| 1054 | 1676 | 1677 | 2500 | 2501 | no gene associated | | See tables 3 & 4 |
| 1055 | 1678 | 1679 | 2502 | 2503 | C20orf112; chromosome 20 open reading | OTTHUMG00000032219 | See tables 3 & 4 |
| | | | | | frame 112; OTTHUMG00000032219 | | |
| 1056 | 1680 | 1681 | 2504 | 2505 | FER1L4; fer-1-like 4 (C. Elegans); | OTTHUMG00000032354 | See tables 3 & 4 |
| | | | | | OTTHUMG00000032354 | | |
| 1057 | 1682 | 1683 | 2506 | 2507 | no gene associated | | See tables 3 & 4 |
| 1058 | 1684 | 1685 | 2508 | 2509 | no gene associated | | See tables 3 & 4 |
| 1059 | 1686 | 1687 | 2510 | 2511 | Protein C20orf102 precursor, | | See tables 3 & 4 |
| | | | | | ENSG00000132821, CT102_HUMAN | | |
| 1060 | 1688 | 1689 | 2512 | 2513 | no gene associated | | See tables 3 & 4 |
| 1061 | 1690 | 1691 | 2514 | 2515 | no gene associated | | See tables 3 & 4 |
| 1062 | 1692 | 1693 | 2516 | 2517 | no gene associated | | See tables 3 & 4 |
| 1063 | 1694 | 1695 | 2518 | 2519 | no gene associated | | See tables 3 & 4 |
| 1064 | 1696 | 1697 | 2520 | 2521 | no gene associated - Nearest transcript | | See tables 3 & 4 |
| | | | | | CDH22 (~18 kb upstream) | | |
| 1065 | 1698 | 1699 | 2522 | 2523 | no gene associated | | See tables 3 & 4 |
| 1066 | 1700 | 1701 | 2524 | 2525 | no gene associated | | See tables 3 & 4 |
| 1067 | 1702 | 1703 | 2526 | 2527 | no gene associated | | See tables 3 & 4 |
| 1068 | 1704 | 1705 | 2528 | 2529 | no gene associated | | See tables 3 & 4 |
| 1069 | 1706 | 1707 | 2530 | 2531 | no gene associated | | See tables 3 & 4 |
| 1070 | 1708 | 1709 | 2532 | 2533 | no gene associated | | See tables 3 & 4 |
| 1071 | 1710 | 1711 | 2534 | 2535 | no gene associated | | See tables 3 & 4 |
| 1072 | 1712 | 1713 | 2536 | 2537 | ZHX3; zinc fingers and homeoboxes 3; | OTTHUMG00000032481 | See tables 3 & 4 |
| | | | | | OTTHUMG00000032481 | | |
| 1073 | 1714 | 1715 | 2538 | 2539 | no gene associated | | See tables 3 & 4 |
| 1074 | 1716 | 1717 | 2540 | 2541 | CHD6; chromodomain helicase DNA | ENSG00000124177 | See tables 3 & 4 |
| | | | | | binding protein 6; ENSG00000124177 | | |
| 1075 | 1718 | 1719 | 2542 | 2543 | no gene associated | | See tables 3 & 4 |
| 1076 | 1720 | 1721 | 2544 | 2545 | PTPRG; protein tyrosine phosphatase, | ENSG00000144724 | See tables 3 & 4 |
| | | | | | receptor type, G; ENSG00000144724 | | |
| 1077 | 1722 | 1723 | 2546 | 2547 | no gene associated | | See tables 3 & 4 |
| 1078 | 1724 | 1725 | 2548 | 2549 | no gene associated | | See tables 3 & 4 |
| 1079 | 1726 | 1727 | 2550 | 2551 | no gene associated | | See tables 3 & 4 |
| 1080 | 1728 | 1729 | 2552 | 2553 | PTPNS1; protein tyrosine phosphatase, non- | ENSG00000198053 | See tables 3 & 4 |
| | | | | | receptor type substrate 1; | | |
| | | | | | ENSG00000198053 | | |
| 1081 | 1730 | 1731 | 2554 | 2555 | Q7Z5T1_HUMAN (Predicted | ENSG00000088881 | See tables 3 & 4 |
| | | | | | UniProt/TrEMBL ID); KIAA1442 protein; | | |
| | | | | | ENSG00000088881 | | |
| 1082 | 1732 | 1733 | 2556 | 2557 | NP_689717.2 (RefSeq peptide ID); | ENSG00000171984 | See tables 3 & 4 |
| | | | | | ENSG00000171984 | | |
| 1083 | 1734 | 1735 | 2558 | 2559 | ENSG00000149346, NP_001009608.1, | ENSG00000149346 | See tables 3 & 4 |
| | | | | | hypothetical protein LOC128710, | | |
| | | | | | chromosome 20 open reading frame 94 | | |
| 1084 | 1736 | 1737 | 2560 | 2561 | C20orf82; chromosome 20 open reading | ENSG00000101230 | See tables 3 & 4 |
| | | | | | frame 82; ENSG00000101230 | | |
| 1085 | 1738 | 1739 | 2562 | 2563 | C20orf23; chromosome 20 open reading | ENSG00000089177 | See tables 3 & 4 |
| | | | | | frame 23; ENSG00000089177; embryonic | | |
| | | | | | marker | | |
| 1086 | 1740 | 1741 | 2564 | 2565 | PCSK2; proprotein convertase | ENSG00000125851 | See tables 3 & 4 |
| | | | | | subtilisin/kexin type 2; ENSG00000125851 | | |
| 1087 | 1742 | 1743 | 2566 | 2567 | PCSK2; proprotein convertase | ENSG00000125851 | See tables 3 & 4 |
| | | | | | subtilisin/kexin type 2; ENSG00000125851 | | |
| 1088 | 1744 | 1745 | 2568 | 2569 | solute carrier family 24 | OTTHUMG00000031993 | See tables 3 & 4 |
| | | | | | (sodiumVpotassiumVcalcium exchanger), | | |
| | | | | | member 3, OTTHUMG00000031993, | | |
| | | | | | SLC24A3 | | |
| 1089 | 1746 | 1747 | 2570 | 2571 | solute carrier family 24 | OTTHUMG00000031993 | See tables 3 & 4 |
| | | | | | (sodiumVpotassiumVcalcium exchanger), | | |
| | | | | | member 3, OTTHUMG00000031993, | | |
| | | | | | SLC24A3 | | |
| 1090 | 1748 | 1749 | 2572 | 2573 | ENSG00000089101, CT026_HUMAN | ENSG00000089101 | See tables 3 & 4 |
| 1091 | 1750 | 1751 | 2574 | 2575 | ENSG00000089101, CT026_HUMAN | ENSG00000089101 | See tables 3 & 4 |
| 1092 | 1752 | 1753 | 2576 | 2577 | C20orf74 protein, ENSG00000188559, | ENSG00000188559 | See tables 3 & 4 |
| | | | | | Q9ULE8_HUMAN | | |
| 1093 | 1754 | 1755 | 2578 | 2579 | C20orf74 protein, ENSG00000188559, | ENSG00000188559 | See tables 3 & 4 |
| | | | | | Q9ULE8_HUMAN | | |
| 1094 | 1756 | 1757 | 2580 | 2581 | C20orf14 protein, ENSG00000188559, | ENSG00000188559 | See tables 3 & 4 |
| | | | | | Q9ULE8_HUMAN | | |
| 1095 | 1758 | 1759 | 2582 | 2583 | PLAGL2; pleiomorphic adenoma gene-like | ENSG00000126003 | See tables 3 & 4 |
| | | | | | 2; ENSG00000126003 | | |
| 1096 | 1760 | 1761 | 2584 | 2585 | GGTL3; gamma-glutamyltransferase-like 3; | ENSG00000131067 | See tables 3 & 4 |
| | | | | | ENSG00000131067 | | |
| 1097 | 1762 | 1763 | 2586 | 2587 | MYH7B; myosin, heavy polypeptide 7B, | ENSG00000078814 | See tables 3 & 4 |
| | | | | | cardiac muscle, beta; ENSG00000078814 | | |
| 1098 | 1764 | 1765 | 2588 | 2589 | TRPC4AP; transient receptor potential cation | ENSG00000100991 | See tables 3 & 4 |
| | | | | | channel, subfamily C, member 4 associated | | |
| | | | | | protein; ENSG00000100991 | | |
| 1099 | 1766 | 1767 | 2590 | 2591 | EPB41L1; erythrocyte membrane protein | ENSG00000088367 | See tables 3 & 4 |
| | | | | | band 4.1-like 1; ENSG00000088367 | | |
| 1100 | 1768 | 1769 | 2592 | 2593 | C20orf117; chromosome 20 open reading | OTTHUMG00000032395 | See tables 3 & 4 |
| | | | | | frame 117; OTTHUMG00000032395 | | |
| 1101 | 1770 | 1771 | 2594 | 2595 | PTPRT; protein tyrosine phosphatase, | ENSG00000196090 | See tables 3 & 4 |
| | | | | | receptor type, T; ENSG00000196090 | | |
| 1102 | 1772 | 1773 | 2596 | 2597 | PTPRT; protein tyrosine phosphatase, | ENSG00000196090 | See tables 3 & 4 |
| | | | | | receptor type, T; ENSG00000196090 | | |
| 1103 | 1774 | 1775 | 2598 | 2599 | PTPRT; protein tyrosine phosphatase, | ENSG00000196090 | See tables 3 & 4 |
| | | | | | receptor type, T; ENSG00000196090 | | |
| 1104 | 1776 | 1777 | 2600 | 2601 | PTPRT; protein tyrosine phosphatase, | ENSG00000196090 | See tables 3 & 4 |
| | | | | | receptor type, T; ENSG00000196090 | | |
| 1105 | 1778 | 1779 | 2602 | 2603 | PTPRT; protein tyrosine phosphatase, | ENSG00000196090 | See tables 3 & 4 |
| | | | | | receptor type, T; ENSG00000196090 | | |
| 1106 | 1780 | 1781 | 2604 | 2605 | SDC4; syndecan 4 (amphiglycan, ryudocan); | ENSG00000124145 | See tables 3 & 4 |
| | | | | | ENSG00000124145 | | |
| 1107 | 1782 | 1783 | 2606 | 2607 | SDC4; syndecan 4 (amphiglycan, ryudocan); | ENSG00000124145 | See tables 3 & 4 |
| | | | | | ENSG00000124145 | | |
| 1108 | 1784 | 1785 | 2608 | 2609 | cadherin-like 22, CDH22 | OTTHUMG00000033073 | See tables 3 & 4 |
| 1109 | 1786 | 1787 | 2610 | 2611 | EYA2; eyes absent homolog 2 (Drosophila); | ENSG00000064655 | See tables 3 & 4 |
| | | | | | ENSG00000064655 | | |
| 1110 | 1788 | 1789 | 2612 | 2613 | SULF2; sulfatase 2; ENSG00000196562 | ENSG00000196562 | See tables 3 & 4 |
| 1111 | 1790 | 1791 | 2614 | 2615 | KCNB1; potassium voltage-gated channel, | ENSG00000158445 | See tables 3 & 4 |
| | | | | | Shab-related subfamily, member 1; | | |
| | | | | | ENSG00000158445 | | |
| 1112 | 1792 | 1793 | 2616 | 2617 | Breast carcinoma amplified sequence 4, | ENSG00000124243 | See tables 3 & 4 |
| | | | | | BCAS4 | | |
| 1113 | 1794 | 1795 | 2618 | 2619 | nuclear factor of activated T-cells, | OTTHUMG00000032747 | See tables 3 & 4 |
| | | | | | cytoplasmic, calcineurin-dependent 2, | | |
| | | | | | OTTHUMG00000032747, NFATC2 | | |
| 1114 | 1796 | 1797 | 2620 | 2621 | Nuclear factor of activated T-cells, | ENSG00000101096 | See tables 3 & 4 |
| | | | | | cytoplasmic 2 (T cell transcription factor | | |
| | | | | | NFAT1) (NFAT pre-existing subunit) (NF- | | |
| | | | | | ATp), NFATC2 | | |
| 1115 | 1798 | 1799 | 2622 | 2623 | Bone morphogenetic protein 7 precursor | ENSG00000101144 | See tables 3 & 4 |
| | | | | | (BMP-7) (Osteogenic protein 1) (OP-1) | | |
| | | | | | (Eptotermin alfa), | | |
| 1116 | 1800 | 1801 | 2624 | 2625 | transmembrane, prostate androgen induced | OTTHUMG00000032831 | See tables 3 & 4 |
| | | | | | RNA, | | |
| 1117 | 1802 | 1803 | 2626 | 2627 | NO annotated gene; NP_775915.1 (RefSeq | ENSG00000176659 | See tables 3 & 4 |
| | | | | | peptide ID) | | |
| 1118 | 1804 | 1805 | 2628 | 2629 | CDH4; cadherin 4, type 1, R-cadherin | ENSG00000179242 | See tables 3 & 4 |
| | | | | | (retinal); ENSG00000179242 | | |
| 1119 | 1806 | 1807 | 2630 | 2631 | NP_001002034.1 (RefSeq peptide ID); | ENSG00000177096 | See tables 3 & 4 |
| | | | | | ENSG00000177096 | | |
| 1120 | 1808 | 1809 | 2632 | 2633 | NP_612444.1 (RefSeq peptide ID); | ENSG00000133477 | See tables 3 & 4 |
| | | | | | ENSG00000133477 | | |
| 1121 | 1810 | 1811 | 2634 | 2635 | no gene associated | | See tables 3 & 4 |
| 1122 | 1812 | 1813 | 2636 | 2637 | OTTHUMG00000030780, CTA-373H7.4, | OTTHUMG00000030780 | See tables 3 & 4 |
| | | | | | novel pseudogene | | |
| 1123 | 1814 | 1815 | 2638 | 2639 | no gene associated | | See tables 3 & 4 |
| 1124 | 1816 | 1817 | 2640 | 2641 | Cat eye syndrome critical region protein 1 | ENSG00000093072 | See tables 3 & 4 |
| | | | | | precursor, CECR1 | | |
| 1125 | 1818 | 1819 | 2642 | 2643 | IGLC1; immunoglobulin lambda constant 1 | ENSG00000100208 | See tables 3 & 4 |
| | | | | | (Mcg marker); ENSG00000100208 | | |
| 1126 | 1820 | 1821 | 2644 | 2645 | OTTHUMG00000030521, AC000095.4 | OTTHUMG00000030521 | See tables 3 & 4 |
| | | | | | putative processed transcript; | | |
| 1127 | 1822 | 1823 | 2646 | 2647 | Uroplakin-3A precursor (Uroplakin III) | ENSG00000100373 | See tables 3 & 4 |
| | | | | | (UPIII)., UPK3A | | |
| 1128 | 1824 | 1825 | 2648 | 2649 | Sp1 site_no gene associated | | See tables 3 & 4 |
| 1129 | 1826 | 1827 | 2650 | 2651 | USP18; ubiquitin specific peptidase 18; | OTTHUMG00000030949 | See tables 3 & 4 |
| | | | | | OTTHUMG00000030949 | | |
| 1130 | 1828 | 1829 | 2652 | 2653 | BCR; breakpoint cluster region; | ENSG00000186716 | See tables 3 & 4 |
| | | | | | ENSG00000186716 | | |
| 1131 | 1830 | 1831 | 2654 | 2655 | TBC1D10A; TBC1 domain family, member | ENSG00000099992 | See tables 3 & 4 |
| | | | | | 10A; ENSG00000099992 | | |
| 1132 | 1832 | 1833 | 2656 | 2657 | signal peptide-CUB domian-EGF-related 1, | ENSG00000159307 | See tables 3 & 4 |
| | | | | | ENSG00000159307, SCUBE1 | | |
| 1133 | 1834 | 1835 | 2658 | 2659 | MAPK8IP2; mitogen-activated protein | ENSG00000008735 | See tables 3 & 4 |
| | | | | | kinase 8 interacting protein 2; | | |
| | | | | | ENSG00000008735 | | |
| 1134 | 1836 | 1837 | 2660 | 2661 | ENSG00000192797, miRNA | ENSG00000192797 | See tables 3 & 4 |
| 1135 | 1838 | 1839 | 2662 | 2663 | RPL3; ribosomal protein L3; | ENSG00000100316 | See tables 3 & 4 |
| | | | | | ENSG00000100316 | | |
| 1136 | 1840 | 1841 | 2664 | 2665 | RPL3; ribosomal protein L3; | ENSG00000100316 | See tables 3 & 4 |
| | | | | | ENSG00000100316 | | |
| 1137 | 1842 | 1843 | 2666 | 2667 | RP4-695O20_B.9 (Vega gene ID); Putative | OTTHUMG00000030111 | See tables 3 & 4 |
| | | | | | Processed transcript; | | |
| | | | | | OTTHUMG00000030111 | | |
| 1138 | 1844 | 1845 | 2668 | 2669 | NOVEL transcript?? No associated gene | | See tables 3 & 4 |
| 1139 | 1846 | 1847 | 2670 | 2671 | MN1; meningioma (disrupted in balanced | ENSG00000169184 | See tables 3 & 4 |
| | | | | | translocation) 1; ENSG00000169184 | | |
| 1140 | 1848 | 1849 | 2672 | 2673 | no gene associated | | See tables 3 & 4 |
| 1141 | 1850 | 1851 | 2674 | 2675 | RTDR1; rhabdoid tumor deletion region gene | ENSG00000100218 | See tables 3 & 4 |
| | | | | | 1; ENSG00000100218 | | |
| 1142 | 1852 | 1853 | 2676 | 2677 | RPL3; ribosomal protein L3; | ENSG00000100316 | See tables 3 & 4 |
| | | | | | ENSG00000100316 | | |
| 1143 | 1854 | 1855 | 2678 | 2679 | embryonic marker, GRB2-related adaptor | OTTHUMG00000030700 | See tables 3 & 4 |
| | | | | | protein 2, OTTHUMG00000030700, GRAP2 | | |
| 1144 | 1856 | 1857 | 2680 | 2681 | Serine/threonine-protein kinase 19 (EC | ENSG00000166301 | See tables 3 & 4 |
| | | | | | 2.7.1.37) (RP1 protein) (G11 protein). | | |
| 1145 | 1858 | 1859 | 2682 | 2683 | Transcription factor 19 (Transcription factor | ENSG00000137310 | See tables 3 & 4 |
| | | | | | SC1). | | |
| 1146 | 1860 | 1861 | 2684 | 2685 | Pannexin-2 | ENSG00000073150 | See tables 3 & 4 |
| 1147 | 1862 | 1863 | 2686 | 2687 | OTTHUMG00000030167 | OTTHUMG00000030167 | See tables 3 & 4 |
| 1148 | 1864 | 1865 | 2688 | 2689 | signal peptide-CUB domian-EGF-related 1 | ENSG00000159307 | See tables 3 & 4 |
| 1149 | 1866 | 1867 | 2690 | 2691 | Reticulon 4 receptor precursor (Nogo | ENSG00000040608 | See tables 3 & 4 |
| | | | | | receptor) (NgR) (Nogo-66 receptor) | | |
| 1150 | 1868 | 1869 | 2692 | 2693 | Arylsulfatase A precursor (EC 3.1.6.8) | ENSG00000100299 | See tables 3 & 4 |
| | | | | | (ASA) (Cerebroside-sulfatase) [Contains: | | |
| | | | | | Arylsulfatase A component B; Arylsulfatase | | |
| | | | | | A component C] | | |
| 1151 | 1870 | 1871 | 2694 | 2695 | glycoprotein Ib (platelet), beta polypeptide | OTTHUMG00000030191 | See tables 3 & 4 |
| 1152 | 1872 | 1873 | 2696 | 2697 | No gene associated | | See tables 3 & 4 |
| 1153 | 1874 | 1875 | 2698 | 2699 | No gene associated | | See tables 3 & 4 |
| 1154 | 1876 | 1877 | 2700 | 2701 | Mitochondrial glutamate carrier 2 | ENSG00000182902 | See tables 3 & 4 |
| | | | | | (Glutamate/H(+) symporter 2) (Solute carrier | | |
| | | | | | family 25 member 18, ENSG00000182902, | | |
| | | | | | SLC25A18 | | |
| 1155 | 1878 | 1879 | 2702 | 2703 | Thioredoxin reductase 2, mitochondrial | ENSG00000184470 | See tables 3 & 4 |
| | | | | | precursor (EC 1.8.1.9) (TR3) (TR-beta) | | |
| | | | | | (Selenoprotein Z) (SelZ) | | |
| 1156 | 1880 | 1881 | 2704 | 2705 | Somatostatin receptor type 3 (SS3R) (SSR- | ENSG00000183473 | See tables 3 & 4 |
| | | | | | 28) | | |
| 1157 | 1882 | 1883 | 2706 | 2707 | OTTHUMG00000030964 | OTTHUMG00000030964 | See tables 3 & 4 |
| 1158 | 1884 | 1885 | 2708 | 2709 | No description-pseudogene | OTTHUMG00000030574 | See tables 3 & 4 |
| 1159 | 1886 | 1887 | 2710 | 2711 | Cat eye syndrome critical region protein 1 | ENST00000262607 | See tables 3 & 4 |
| | | | | | precursor | | |
| 1160 | 1888 | 1889 | 2712 | 2713 | No gene associated | | See tables 3 & 4 |
| 1161 | 1890 | 1891 | 2714 | 2715 | Membrane protein MLC1 | ENSG00000100427 | See tables 3 & 4 |
| 1162 | 1892 | 1893 | 2716 | 2717 | BAI1-associated protein 2-like 2 | ENSG00000128298 | See tables 3 & 4 |
| 1163 | 1894 | 1895 | 2718 | 2719 | ENSG00000100249 | ENSG00000100249 | See tables 3 & 4 |
| 1164 | 1896 | 1897 | 2720 | 2721 | OTTHUMG00000030111 | OTTHUMG00000030111 | See tables 3 & 4 |
| 1165 | 1898 | 1899 | 2722 | 2723 | OTTHUMG00000030167, CTA-243E7.3 | OTTHUMG00000030167 | See tables 3 & 4 |
| 1166 | 1900 | 1901 | 2724 | 2725 | OTTHUMG00000030620 | OTTHUMG00000030620 | See tables 3 & 4 |
| 1167 | 1902 | 1903 | 2726 | 2727 | OTTHUMG00000030676 | OTTHUMG00000030676 | See tables 3 & 4 |
| 1168 | 1904 | 1905 | 2728 | 2729 | ENSG00000197549 | ENSG00000197549 | See tables 3 & 4 |
| 1169 | 1906 | 1907 | 2730 | 2731 | NFAT activation molecule 1 precursor | ENSG00000167087 | See tables 3 & 4 |
| | | | | | (Calcineurin/NFAT-activating ITAM- | | |
| | | | | | containing protein) (NFAT activating protein | | |
| | | | | | with ITAM motif 1). | | |
| 1170 | 1908 | 1909 | 2732 | 2733 | immunoglobulin lambda constant 2 | OTTHUMG00000030352 | See tables 3 & 4 |
| 1171 | 1910 | 1911 | 2734 | 2735 | immunoglobulin lambda constant 2 | OTTHUMG00000030352 | See tables 3 & 4 |
| 1172 | 1912 | 1913 | 2736 | 2737 | OTTHUMG00000030870, CTA-503F6.1 | OTTHUMG00000030870 | See tables 3 & 4 |
| 1173 | 1914 | 1915 | 2738 | 2739 | Lactosylceramide 4-alpha- | ENSG00000128274 | See tables 3 & 4 |
| | | | | | galactosyltransferase (EC 2.4.1.228) | | |
| 1174 | 1916 | 1917 | 2740 | 2741 | OTTHUMG00000030966 | OTTHUMG00000030966 | See tables 3 & 4 |
| 1175 | 1918 | 1919 | 2742 | 2743 | Cold shock domain protein C2 (RNA- | ENSG00000172346 | See tables 3 & 4 |
| | | | | | binding protein PIPPin) | | |
| 1176 | 1920 | 1921 | 2744 | 2745 | GAS2-like protein 1 (Growth arrest-specific | ENSG00000185340 | See tables 3 & 4 |
| | | | | | 2-like 1) (GAS2-related protein on | | |
| | | | | | chromosome 22) (GAR22 protein), GAS2L1 | | |
| 1177 | 1922 | 1923 | 2746 | 2747 | BAI1-associated protein 2-like 2 | ENSG00000128298 | See tables 3 & 4 |
| 1178 | 1924 | 1925 | 2748 | 2749 | ENSG00000197182 | ENSG00000197182 | See tables 3 & 4 |
| 1179 | 1926 | 1927 | 2750 | 2751 | OTTHUMG00000030991, LL22NC03- | OTTHUMG00000030991 | See tables 3 & 4 |
| | | | | | 75B3.6 | | |
| 1180 | 1928 | 1929 | 2752 | 2753 | Reticulon 4 receptor precursor (Nogo | ENSG00000040608 | See tables 3 & 4 |
| | | | | | receptor) (NgR) (Nogo-66 receptor) | | |
| 1181 | 1930 | 1931 | 2754 | 2755 | Smoothelin; SMTN | ENSG00000183963 | See tables 3 & 4 |
| 1182 | 1932 | 1933 | 2756 | 2757 | solute carrier family 35, member E4 | ENSG00000100036 | See tables 3 & 4 |
| 1183 | 1934 | 1935 | 2758 | 2759 | Protein C22orf13 (Protein LLN4) | ENSG00000138867 | See tables 3 & 4 |
| 1184 | 1936 | 1937 | 2760 | 2761 | No gene associated | | See tables 3 & 4 |
| 1185 | 1938 | 1939 | 2762 | 2763 | Histone | ENSG00000196966 | See tables 3 & 4 |
| 1186 | 1940 | 1941 | 2764 | 2765 | Gamma-aminobutyric-acid receptor rho-1 | ENSG00000146276 | See tables 3 & 4 |
| | | | | | subunit precursor (GABA(A) receptor). | | |
| 1187 | 1942 | 1943 | 2766 | 2767 | OTTHUMG00000015693, RP11-12A2.3 | OTTHUMG00000015693 | See tables 3 & 4 |
| 1188 | 1944 | 1945 | 2768 | 2769 | OTTHUMG00000015697 | OTTHUMG00000015697 | See tables 3 & 4 |
| 1189 | 1946 | 1947 | 2770 | 2771 | OTTHUMG00000014289 | OTTHUMG00000014289 | See tables 3 & 4 |
| 1190 | 1948 | 1949 | 2772 | 2773 | ENSG00000178289 | ENSG00000178289 | See tables 3 & 4 |
| 1191 | 1950 | 1951 | 2774 | 2775 | Forkhead box protein O3A, | ENSG00000118689 | See tables 3 & 4 |
| 1192 | 1952 | 1953 | 2776 | 2777 | nuclear receptor coactivator 7 | ENSG00000111912 | See tables 3 & 4 |
| 1193 | 1954 | 1955 | 2778 | 2779 | OTTHUMG00000015043 | OTTHUMG00000015043 | See tables 3 & 4 |
| 1194 | 1956 | 1957 | 2780 | 2781 | chromosome 6 open reading frame 190 | OTTHUMG00000015534 | See tables 3 & 4 |
| 1195 | 1958 | 1959 | 2782 | 2783 | phosphatase and actin regulator 2 | OTTHUMG00000015732 | See tables 3 & 4 |
| 1196 | 1960 | 1961 | 2784 | 2785 | High mobility group protein HMG-I/HMG-Y | ENSG00000137309 | See tables 3 & 4 |
| | | | | | (HMG-I(Y)) (High mobility group AT-hook | | |
| | | | | | 1) (High mobility group protein A1), | | |
| 1197 | 1962 | 1963 | 2786 | 2787 | Pantetheinase precursor (EC 3.5.1.—), | ENSG00000112299 | See tables 3 & 4 |
| | | | | | ENSG00000112299, VNN1 | | |
| 1198 | 1964 | 1965 | 2788 | 2789 | histone H2A | ENSG00000164508 | See tables 3 & 4 |
| 1199 | 1966 | 1967 | 2790 | 2791 | transcription factor AP-2 alpha (activating | OTTHUMG00000014235 | See tables 3 & 4 |
| | | | | | enhancer binding protein 2 alpha) | | |
| 1200 | 1968 | 1969 | 2792 | 2793 | N-acetyllactosaminide beta-1,6-N- | ENSG00000111846 | See tables 3 & 4 |
| | | | | | acetylglucosaminyl-transferase (EC | | |
| | | | | | 2.4.1.150), ENSG00000111846, GCNT2 | | |
| 1201 | 1970 | 1971 | 2794 | 2795 | No gene associated | | See tables 3 & 4 |
| 1202 | 1972 | 1973 | 2796 | 2797 | No gene associated | | See tables 3 & 4 |
| 1203 | 1974 | 1975 | 2798 | 2799 | No gene associated | | See tables 3 & 4 |
| 1204 | 1976 | 1977 | 2800 | 2801 | No gene associated | | See tables 3 & 4 |
| 1205 | 1978 | 1979 | 2802 | 2803 | No gene associated | | See tables 3 & 4 |
| 1206 | 1980 | 1981 | 2804 | 2805 | No gene associated | | See tables 3 & 4 |
| 1207 | 1982 | 1983 | 2806 | 2807 | No gene associated | | See tables 3 & 4 |
| 1208 | 1984 | 1985 | 2808 | 2809 | No gene associated | | See tables 3 & 4 |
| 1209 | 1986 | 1987 | 2810 | 2811 | No gene associated | | See tables 3 & 4 |
| 1210 | 1988 | 1989 | 2812 | 2813 | No gene associated | | See tables 3 & 4 |
| 1211 | 1990 | 1991 | 2814 | 2815 | No description | OTTHUMG00000031920 | See tables 3 & 4 |
| 1212 | 1992 | 1993 | 2816 | 2817 | No gene associated | | See tables 3 & 4 |
| 1213 | 1994 | 1995 | 2818 | 2819 | No gene associated | | See tables 3 & 4 |
| 1214 | 1996 | 1997 | 2820 | 2821 | No gene associated | | See tables 3 & 4 |
| 1215 | 1998 | 1999 | 2822 | 2823 | No gene associated | | See tables 3 & 4 |
| 1216 | 2000 | 2001 | 2824 | 2825 | No gene associated | | See tables 3 & 4 |
| 1217 | 2002 | 2003 | 2826 | 2827 | No gene associated | | See tables 3 & 4 |
| 1218 | 2004 | 2005 | 2828 | 2829 | OTTHUMG00000032045 | OTTHUMG00000032045 | See tables 3 & 4 |
| 1219 | 2006 | 2007 | 2830 | 2831 | No gene associated | | See tables 3 & 4 |
| 1220 | 2008 | 2009 | 2832 | 2833 | No gene associated | | See tables 3 & 4 |
| 1221 | 2010 | 2011 | 2834 | 2835 | No gene associated | | See tables 3 & 4 |
| 1222 | 2012 | 2013 | 2836 | 2837 | OTTHUMG00000032221 | OTTHUMG00000032221 | See tables 3 & 4 |
| 1223 | 2014 | 2015 | 2838 | 2839 | TIMP3 | ENSG00000100234 | See tables 3 & 4 |
| 1224 | 2016 | 2017 | 2840 | 2841 | No gene associated | | See tables 3 & 4 |
| 1225 | 2018 | 2019 | 2842 | 2843 | No gene associated | | See tables 3 & 4 |
| 1226 | 2020 | 2021 | 2844 | 2845 | No gene associated | | See tables 3 & 4 |
| 1227 | 2022 | 2023 | 2846 | 2847 | No gene associated | | See tables 3 & 4 |
| 1228 | 2024 | 2025 | 2848 | 2849 | no gene associated | | See tables 3 & 4 |
| 1229 | 2026 | 2027 | 2850 | 2851 | No gene associated | | See tables 3 & 4 |
| 1230 | 2028 | 2029 | 2852 | 2853 | No gene associated | | See tables 3 & 4 |
| 1231 | 2030 | 2031 | 2854 | 2855 | No gene associated | | See tables 3 & 4 |
| 1232 | 2032 | 2033 | 2856 | 2857 | No gene associated | | See tables 3 & 4 |
| 1233 | 2034 | 2035 | 2858 | 2859 | No gene associated | | See tables 3 & 4 |
| 1234 | 2036 | 2037 | 2860 | 2861 | No gene associated | | See tables 3 & 4 |
| 1235 | 2038 | 2039 | 2862 | 2863 | sorting nexin 5 | OTTHUMG00000031953 | See tables 3 & 4 |
| 1236 | 2040 | 2041 | 2864 | 2865 | Probable D-tyrosyl-tRNA(Tyr) deacylase | ENSG00000125821 | See tables 3 & 4 |
| | | | | | (EC 3.1.—.—) | | |
| 1237 | 2042 | 2043 | 2866 | 2867 | solute carrier family 24 | OTTHUMG00000031993 | See tables 3 & 4 |
| | | | | | (sodiumVpotassiumVcalcium exchanger), | | |
| | | | | | member 3, OTTHUMG00000031993, | | |
| | | | | | SLC24A3 | | |
| 1238 | 2044 | 2045 | 2868 | 2869 | ENSG00000089101 | ENSG00000089101 | See tables 3 & 4 |
| 1239 | 2046 | 2047 | 2870 | 2871 | RNA-binding protein Raly (hnRNP | ENSG00000125970 | See tables 3 & 4 |
| | | | | | associated with lethal yellow homolog), D; | | |
| | | | | | RALY | | |
| 1240 | 2048 | 2049 | 2872 | 2873 | Protein phosphatase 1 regulatory inhibitor | ENSG00000101445 | See tables 3 & 4 |
| | | | | | subunit 16B (TGF-beta-inhibited membrane- | | |
| | | | | | associated protein) (hTIMAP) (CAAX box | | |
| | | | | | protein TIMAP) (Ankyrin repeat domain | | |
| | | | | | protein 4) | | |
| 1241 | 2050 | 2051 | 2874 | 2875 | protein tyrosine phosphatase, receptor type, T | OTTHUMG00000033040 | See tables 3 & 4 |
| 1242 | 2052 | 2053 | 2876 | 2877 | protein tyrosine phosphatase, receptor type, T | OTTHUMG00000033040 | See tables 3 & 4 |
| 1243 | 2054 | 2055 | 2878 | 2879 | protein tyrosine phosphatase, receptor type, T | OTTHUMG00000033040 | See tables 3 & 4 |
| 1244 | 2056 | 2057 | 2880 | 2881 | Receptor-type tyrosine-protein phosphatase T | ENSG00000196090 | See tables 3 & 4 |
| | | | | | precursor (EC 3.1.3.48) (R-PTP-T) (RPTP- | | |
| | | | | | rho) | | |
| 1245 | 2058 | 2059 | 2882 | 2883 | cadherin-like 22 | OTTHUMG00000033073 | See tables 3 & 4 |
| 1246 | 2060 | 2061 | 2884 | 2885 | potassium voltage-gated channel, Shab- | OTTHUMG00000033051 | See tables 3 & 4 |
| | | | | | related subfamily, member 1 | | |
| 1247 | 2062 | 2063 | 2886 | 2887 | potassium voltage-gated channel, Shab- | OTTHUMG00000033051 | See tables 3 & 4 |
| | | | | | related subfamily, member 1 | | |
| 1248 | 2064 | 2065 | 2888 | 2889 | Zinc finger protein SNAI1 (Snail protein | ENSG00000124216 | See tables 3 & 4 |
| | | | | | homolog) (Sna protein) | | |
| 1249 | 2066 | 2067 | 2890 | 2891 | Cadherin-4 precursor (Retinal-cadherin) (R- | ENSG00000179242 | See tables 3 & 4 |
| | | | | | cadherin) (R-CAD) | | |
| 1250 | 2068 | 2069 | 2892 | 2893 | cadherin 4, type 1, R-cadherin (retinal) | OTTHUMG00000032890 | See tables 3 & 4 |
| 1251 | 2070 | 2071 | 2894 | 2895 | Cadherin-4 precursor (Retinal-cadherin) (R- | ENSG00000179242 | See tables 3 & 4 |
| | | | | | cadherin) (R-CAD) | | |
| 1252 | 2072 | 2073 | 2896 | 2897 | Metalloproteinase inhibitor 3 precursor | | See tables 3 & 4 |
| | | | | | (TIMP-3) (Tissue inhibitor of | | |
| | | | | | metalloproteinases-3) (MIG-5 protein). | | |
| 1253 | 2074 | 2075 | 2898 | 2899 | Tubulin alpha-8 chain (Alpha-tubulin 8) | ENSG00000070490 | See tables 3 & 4 |
| 1254 | 2076 | 2077 | 2900 | 2901 | No gene associated | | See tables 3 & 4 |
| 1255 | 2078 | 2079 | 2902 | 2903 | No gene associated | | See tables 3 & 4 |

TABLE 3
Characteristic methylation value ranges of tissue markers according to the present invention

| | | | | Embryonic | | |
|---|---|---|---|---|---|---|
| SEQ ID | CD4 T- | CD8 T- | Embryonic | Skeletal | | Heart |
| NO: Genomic | lymphocyte | lymphocyte | Liver | Muscle | Fibroblast | Muscle |
| 844 | 75-100% | 75-100% | 75-100% | 75-100% | 75-100% | 75-100% |
| 845 | 75-100% | 75-100% | 75-100% | 75-100% | 75-100% | 75-100% |
| 846 | 75-100% | 75-100% | 75-100% | 75-100% | 75-100% | 75-100% |
| 847 | 75-100% | 75-100% | 0-25% | 0-25% | 0-25% | 0-25% |
| 848 | 0-25% | 0-25% | 75-100% | 75-100% | 75-100% | 75-100% |
| 849 | 75-100% | 75-100% | 75-100% | 75-100% | 75-100% | 75-100% |
| 850 | 75-100% | 75-100% | 75-100% | 75-100% | 75-100% | 75-100% |
| 851 | 75-100% | 75-100% | 75-100% | 0-25% | 75-100% | 75-100% |
| 852 | 75-100% | 75-100% | 75-100% | 75-100% | 75-100% | 75-100% |
| 853 | 25-75% | 25-75% | 25-75% | 25-75% | 0-25% | 25-75% |
| 854 | 0-25% | 0-25% | 0-25% | 0-25% | 0-25% | 0-25% |
| 855 | 75-100% | 75-100% | 75-100% | 25-75% | 75-100% | 25-75% |
| 856 | 75-100% | 75-100% | 25-75% | 25-75% | 25-75% | 25-75% |
| 857 | 25-75% | 25-75% | 0-25% | 0-25% | 0-25% | 0-25% |
| 858 | 75-100% | 75-100% | 75-100% | 75-100% | 75-100% | 75-100% |
| 859 | 75-100% | 75-100% | 75-100% | 75-100% | 75-100% | 75-100% |
| 860 | 0-25% | 0-25% | 0-25% | 75-100% | 75-100% | 25-75% |
| 861 | 75-100% | 75-100% | 75-100% | 75-100% | 75-100% | 25-75% |
| 862 | 75-100% | 75-100% | 75-100% | 75-100% | 75-100% | 75-100% |
| 863 | 75-100% | 75-100% | 75-100% | 75-100% | 75-100% | 75-100% |
| 864 | 75-100% | 75-100% | 75-100% | 75-100% | 75-100% | 75-100% |
| 865 | 75-100% | 75-100% | 75-100% | 75-100% | 75-100% | 75-100% |
| 866 | 75-100% | 75-100% | 75-100% | 75-100% | 75-100% | 75-100% |
| 867 | 75-100% | 75-100% | 75-100% | 75-100% | 75-100% | 75-100% |
| 868 | 75-100% | 75-100% | 75-100% | 75-100% | 75-100% | 75-100% |
| 869 | 75-100% | 75-100% | 25-75% | 25-75% | 25-75% | 25-75% |
| 870 | 25-75% | 25-75% | 25-75% | 25-75% | 25-75% | 25-75% |
| 871 | 75-100% | 75-100% | 75-100% | 75-100% | 0-25% | 75-100% |
| 872 | 75-100% | 75-100% | 75-100% | 25-75% | 75-100% | 75-100% |
| 873 | 75-100% | 75-100% | 25-75% | 25-75% | 25-75% | 25-75% |
| 874 | 75-100% | 75-100% | 75-100% | 75-100% | 75-100% | 75-100% |
| 875 | 75-100% | 75-100% | 75-100% | 25-75% | 75-100% | 75-100% |
| 876 | 75-100% | 75-100% | 0-25% | 0-25% | 0-25% | 0-25% |
| 877 | 75-100% | 75-100% | 75-100% | 75-100% | 0-25% | 75-100% |
| 878 | 75-100% | 75-100% | 75-100% | 75-100% | 75-100% | 75-100% |
| 879 | 25-75% | 25-75% | 25-75% | 25-75% | 25-75% | 25-75% |
| 880 | 0-25% | 0-25% | 75-100% | 75-100% | 75-100% | 75-100% |
| 881 | 25-75% | 25-75% | 25-75% | 25-75% | 75-100% | 75-100% |
| 882 | 0-25% | 0-25% | 75-100% | 75-100% | 75-100% | 75-100% |
| 883 | 0-25% | 0-25% | 75-100% | 75-100% | 75-100% | 75-100% |
| 884 | 75-100% | 75-100% | 75-100% | 75-100% | 75-100% | 75-100% |
| 885 | 75-100% | 75-100% | 25-75% | 25-75% | 25-75% | 25-75% |
| 886 | 75-100% | 75-100% | 75-100% | 75-100% | 75-100% | 75-100% |
| 887 | 75-100% | 75-100% | 75-100% | 75-100% | 75-100% | 75-100% |
| 888 | 75-100% | 75-100% | 75-100% | 25-75% | 75-100% | 75-100% |
| 889 | 0-25% | 0-25% | 75-100% | 75-100% | 75-100% | 75-100% |
| 890 | 75-100% | 75-100% | 0-25% | 75-100% | 75-100% | 75-100% |
| 891 | 75-100% | 75-100% | 75-100% | 75-100% | 75-100% | 75-100% |
| 892 | 75-100% | 75-100% | 75-100% | 75-100% | 75-100% | 75-100% |
| 893 | 75-100% | 75-100% | 75-100% | 75-100% | 25-75% | 75-100% |
| 894 | 0-25% | 0-25% | 25-75% | 25-75% | 25-75% | 25-75% |
| 895 | 75-100% | 75-100% | 75-100% | 75-100% | 75-100% | 75-100% |
| 896 | 75-100% | 75-100% | 75-100% | 0-25% | 0-25% | 0-25% |
| 897 | 75-100% | 75-100% | 75-100% | 75-100% | 75-100% | 75-100% |
| 898 | 75-100% | 75-100% | 25-75% | 25-75% | 25-75% | 25-75% |
| 899 | 75-100% | 75-100% | 25-75% | 25-75% | 0-25% | 25-75% |
| 900 | 75-100% | 75-100% | 75-100% | 75-100% | 75-100% | 75-100% |
| 901 | 0-25% | 0-25% | 0-25% | 0-25% | 0-25% | 0-25% |
| 902 | 75-100% | 75-100% | 75-100% | 75-100% | 25-75% | 25-75% |
| 903 | 75-100% | 75-100% | 75-100% | 75-100% | 75-100% | 75-100% |
| 904 | 75-100% | 75-100% | 75-100% | 75-100% | 25-75% | 75-100% |
| 905 | 0-25% | 0-25% | 75-100% | 75-100% | 75-100% | 75-100% |
| 906 | 75-100% | 75-100% | 25-75% | 75-100% | 75-100% | 75-100% |
| 907 | 75-100% | 75-100% | 25-75% | 25-75% | 75-100% | 75-100% |
| 908 | 75-100% | 75-100% | 25-75% | 0-25% | 0-25% | 25-75% |
| 909 | 75-100% | 75-100% | 25-75% | 25-75% | 25-75% | 25-75% |
| 910 | 75-100% | 75-100% | 25-75% | 0-25% | 0-25% | 25-75% |
| 911 | 25-75% | 25-75% | 25-75% | 75-100% | 75-100% | 75-100% |
| 912 | 75-100% | 75-100% | 25-75% | 25-75% | 25-75% | 25-75% |
| 913 | 75-100% | 75-100% | 25-75% | 25-75% | 25-75% | 25-75% |
| 914 | 75-100% | 75-100% | 25-75% | 25-75% | 25-75% | 25-75% |
| 915 | 75-100% | 75-100% | 25-75% | 0-25% | 0-25% | 25-75% |
| 916 | 75-100% | 75-100% | 75-100% | 0-25% | 0-25% | 0-25% |
| 917 | 0-25% | 0-25% | 0-25% | 0-25% | 0-25% | 0-25% |
| 918 | 75-100% | 75-100% | 75-100% | 25-75% | 25-75% | 75-100% |
| 919 | 75-100% | 75-100% | 75-100% | 25-75% | 75-100% | 75-100% |
| 920 | 0-25% | 0-25% | 25-75% | 25-75% | 25-75% | 25-75% |
| 921 | 0-25% | 0-25% | 75-100% | 75-100% | 75-100% | 75-100% |
| 922 | 75-100% | 75-100% | 0-25% | 0-25% | 0-25% | 0-25% |
| 923 | 75-100% | 75-100% | 0-25% | 0-25% | 0-25% | 0-25% |
| 924 | 0-25% | 0-25% | 25-75% | 25-75% | 75-100% | 75-100% |
| 925 | 75-100% | 75-100% | 75-100% | 75-100% | 75-100% | 75-100% |
| 926 | 75-100% | 75-100% | 75-100% | 75-100% | 75-100% | 75-100% |
| 927 | 75-100% | 75-100% | 25-75% | 25-75% | 25-75% | 25-75% |
| 928 | 0-25% | 0-25% | 75-100% | 75-100% | 75-100% | 75-100% |
| 929 | 75-100% | 75-100% | 75-100% | 75-100% | 75-100% | 75-100% |
| 930 | 75-100% | 75-100% | 75-100% | 75-100% | 75-100% | 75-100% |
| 931 | 75-100% | 75-100% | 75-100% | 75-100% | 75-100% | 75-100% |
| 932 | 25-75% | 25-75% | 25-75% | 25-75% | 25-75% | 25-75% |
| 933 | 25-75% | 25-75% | 25-75% | 25-75% | 25-75% | 25-75% |
| 934 | 75-100% | 75-100% | 75-100% | 75-100% | 75-100% | 75-100% |
| 935 | 75-100% | 75-100% | 75-100% | 75-100% | 0-25% | 75-100% |
| 936 | 0-25% | 0-25% | 75-100% | 0-25% | 0-25% | 0-25% |
| 937 | 75-100% | 75-100% | 75-100% | 75-100% | 75-100% | 75-100% |
| 938 | 75-100% | 75-100% | 75-100% | 75-100% | 75-100% | 75-100% |
| 939 | 0-25% | 0-25% | 0-25% | 0-25% | 0-25% | 0-25% |
| 940 | 75-100% | 75-100% | 25-75% | 75-100% | 75-100% | 75-100% |
| 941 | 75-100% | 75-100% | 75-100% | 75-100% | 75-100% | 75-100% |
| 942 | 25-75% | 75-100% | 75-100% | 75-100% | 75-100% | 25-75% |
| 943 | 75-100% | 75-100% | 75-100% | 75-100% | 75-100% | 75-100% |
| 944 | 25-75% | 25-75% | 75-100% | 75-100% | 75-100% | 75-100% |
| 945 | 75-100% | 75-100% | 75-100% | 75-100% | 75-100% | 75-100% |
| 946 | 0-25% | 0-25% | 75-100% | 75-100% | 75-100% | 75-100% |
| 947 | 75-100% | 75-100% | 75-100% | 75-100% | 75-100% | 75-100% |
| 948 | 75-100% | 75-100% | 75-100% | 75-100% | 75-100% | 75-100% |
| 949 | 75-100% | 75-100% | 25-75% | 25-75% | 25-75% | 25-75% |
| 950 | 0-25% | 0-25% | 25-75% | 25-75% | 25-75% | 25-75% |
| 951 | 75-100% | 75-100% | 75-100% | 75-100% | 75-100% | 75-100% |
| 952 | 75-100% | 75-100% | 75-100% | 75-100% | 75-100% | 75-100% |
| 953 | 0-25% | 0-25% | 75-100% | 75-100% | 75-100% | 75-100% |
| 954 | 75-100% | 75-100% | 75-100% | 75-100% | 75-100% | 75-100% |
| 955 | 75-100% | 75-100% | 75-100% | 75-100% | 0-25% | 75-100% |
| 956 | 0-25% | 0-25% | ND | 0-25% | 75-100% | 0-25% |
| 957 | 75-100% | 75-100% | 75-100% | 75-100% | 75-100% | 75-100% |
| 958 | 75-100% | 75-100% | 0-25% | 0-25% | 0-25% | 0-25% |
| 959 | 0-25% | 0-25% | 75-100% | 75-100% | 75-100% | 75-100% |
| 960 | 75-100% | 75-100% | 75-100% | 75-100% | 75-100% | 75-100% |
| 961 | 75-100% | 75-100% | 75-100% | 75-100% | 75-100% | 75-100% |
| 962 | 0-25% | 0-25% | 25-75% | 25-75% | 25-75% | 25-75% |
| 963 | 75-100% | 75-100% | 75-100% | 75-100% | 75-100% | 75-100% |
| 964 | 75-100% | 75-100% | 75-100% | 75-100% | 75-100% | 75-100% |
| 965 | 75-100% | 75-100% | 75-100% | 75-100% | 25-75% | 75-100% |
| 966 | 0-25% | 0-25% | 75-100% | 75-100% | 75-100% | 75-100% |
| 967 | 75-100% | 75-100% | 75-100% | 75-100% | 75-100% | 75-100% |
| 968 | 25-75% | 25-75% | 0-25% | 0-25% | 0-25% | 25-75% |
| 969 | 0-25% | 0-25% | 0-25% | 0-25% | 0-25% | 0-25% |
| 970 | 75-100% | 75-100% | 75-100% | 75-100% | 75-100% | 75-100% |
| 971 | 75-100% | 75-100% | 75-100% | 75-100% | 75-100% | 75-100% |
| 972 | 25-75% | 25-75% | 25-75% | 25-75% | 25-75% | 25-75% |
| 973 | 75-100% | 75-100% | 25-75% | 25-75% | 25-75% | 25-75% |
| 974 | 75-100% | 75-100% | 75-100% | 75-100% | 75-100% | 75-100% |
| 975 | 75-100% | 75-100% | 75-100% | 75-100% | 75-100% | 75-100% |
| 976 | 0-25% | 0-25% | 0-25% | 0-25% | 0-25% | 0-25% |
| 977 | 0-25% | 0-25% | 0-25% | 0-25% | 0-25% | 0-25% |
| 978 | 75-100% | 75-100% | 75-100% | 75-100% | 75-100% | 75-100% |
| 979 | 75-100% | 75-100% | 75-100% | 75-100% | 75-100% | 75-100% |
| 980 | 75-100% | 75-100% | 75-100% | 75-100% | 75-100% | 75-100% |
| 981 | 75-100% | 75-100% | 75-100% | 75-100% | 75-100% | 75-100% |
| 982 | 75-100% | 75-100% | 75-100% | 75-100% | 75-100% | 75-100% |
| 983 | 75-100% | 75-100% | 25-75% | 75-100% | 75-100% | 75-100% |
| 984 | 75-100% | 75-100% | 75-100% | 75-100% | 75-100% | 75-100% |
| 985 | 0-25% | 0-25% | 75-100% | 75-100% | 75-100% | 75-100% |
| 986 | 75-100% | 75-100% | 0-25% | 0-25% | 0-25% | 0-25% |
| 987 | 75-100% | 75-100% | 75-100% | 75-100% | 75-100% | 75-100% |
| 988 | 75-100% | 75-100% | 25-75% | 25-75% | 25-75% | 25-75% |
| 989 | 75-100% | 75-100% | 0-25% | 0-25% | 0-25% | 0-25% |
| 990 | 75-100% | 75-100% | 75-100% | 75-100% | 75-100% | 75-100% |
| 991 | 75-100% | 75-100% | 75-100% | 75-100% | 75-100% | 75-100% |
| 992 | 75-100% | 75-100% | 75-100% | 75-100% | 75-100% | 25-75% |
| 993 | 0-25% | 0-25% | 75-100% | 75-100% | 75-100% | 75-100% |
| 994 | 75-100% | 75-100% | 75-100% | 75-100% | 25-75% | 75-100% |
| 995 | 25-75% | 25-75% | 25-75% | 25-75% | 75-100% | 25-75% |
| 996 | 75-100% | 75-100% | 75-100% | 75-100% | 75-100% | 75-100% |
| 997 | 75-100% | 75-100% | ND | 25-75% | 0-25% | 25-75% |
| 998 | 75-100% | 75-100% | 75-100% | 75-100% | 25-75% | 75-100% |
| 999 | 0-25% | 0-25% | 0-25% | 0-25% | 0-25% | 0-25% |
| 1000 | 75-100% | 75-100% | 75-100% | 75-100% | 0-25% | 25-75% |
| 1001 | 75-100% | 75-100% | 75-100% | 75-100% | 75-100% | 75-100% |
| 1002 | 75-100% | 75-100% | 25-75% | 25-75% | 25-75% | 25-75% |
| 1003 | 25-75% | 25-75% | 25-75% | 25-75% | 75-100% | 25-75% |
| 1004 | 75-100% | 75-100% | 75-100% | 75-100% | 75-100% | 75-100% |
| 1005 | 0-25% | 0-25% | 75-100% | 75-100% | 75-100% | 75-100% |
| 1006 | 75-100% | 75-100% | 75-100% | 75-100% | 75-100% | 75-100% |
| 1007 | 0-25% | 0-25% | 0-25% | 0-25% | 75-100% | 0-25% |
| 1008 | 75-100% | 75-100% | ND | 75-100% | 75-100% | 75-100% |
| 1009 | 75-100% | 75-100% | 75-100% | 75-100% | 75-100% | 75-100% |
| 1010 | 0-25% | 0-25% | 0-25% | 0-25% | 0-25% | 25-75% |
| 1011 | 75-100% | 75-100% | 75-100% | 75-100% | 0-25% | 75-100% |
| 1012 | 75-100% | 75-100% | 75-100% | 75-100% | 75-100% | 75-100% |
| 1013 | 75-100% | 75-100% | 75-100% | 75-100% | 75-100% | 75-100% |
| 1014 | 75-100% | 75-100% | 75-100% | 75-100% | 75-100% | 75-100% |
| 1015 | 0-25% | 0-25% | 0-25% | 0-25% | 75-100% | 0-25% |
| 1016 | 75-100% | 75-100% | 75-100% | 75-100% | 75-100% | 75-100% |
| 1017 | 75-100% | 75-100% | 75-100% | 75-100% | 0-25% | 75-100% |
| 1018 | 75-100% | 75-100% | 75-100% | 75-100% | 75-100% | 75-100% |
| 1019 | 75-100% | 75-100% | 25-75% | 0-25% | 0-25% | 25-75% |
| 1020 | 0-25% | 0-25% | 0-25% | 0-25% | 0-25% | 0-25% |
| 1021 | 25-75% | 25-75% | 0-25% | 0-25% | 0-25% | 25-75% |
| 1022 | 0-25% | 0-25% | 0-25% | 0-25% | 0-25% | 0-25% |
| 1023 | 75-100% | 75-100% | 75-100% | 75-100% | 75-100% | 75-100% |
| 1024 | 75-100% | 75-100% | 25-75% | 0-25% | 0-25% | 25-75% |
| 1025 | 75-100% | 75-100% | 75-100% | 75-100% | 0-25% | 75-100% |
| 1026 | 75-100% | 75-100% | 75-100% | 75-100% | 75-100% | 25-75% |
| 1027 | 75-100% | 75-100% | 75-100% | 25-75% | 0-25% | 75-100% |
| 1028 | 75-100% | 75-100% | 0-25% | 0-25% | 0-25% | 0-25% |
| 1029 | 0-25% | 0-25% | 75-100% | 75-100% | 75-100% | 75-100% |
| 1030 | 75-100% | 75-100% | 75-100% | 75-100% | 25-75% | 75-100% |
| 1031 | 75-100% | 75-100% | 75-100% | 75-100% | 75-100% | 75-100% |
| 1032 | 75-100% | 75-100% | 75-100% | 75-100% | 25-75% | 75-100% |
| 1033 | 75-100% | 75-100% | 75-100% | 75-100% | 75-100% | 75-100% |
| 1034 | 25-75% | 75-100% | 75-100% | 75-100% | 75-100% | 75-100% |
| 1035 | 75-100% | 75-100% | 75-100% | 75-100% | 75-100% | 75-100% |
| 1036 | 75-100% | 75-100% | 75-100% | 75-100% | 75-100% | 75-100% |
| 1037 | 75-100% | 75-100% | 75-100% | 75-100% | 75-100% | 75-100% |
| 1038 | 0-25% | 0-25% | 75-100% | 75-100% | 75-100% | 75-100% |
| 1039 | 75-100% | 75-100% | 75-100% | 75-100% | 75-100% | 75-100% |
| 1040 | 75-100% | ND | ND | ND | 0-25% | 75-100% |
| 1041 | 75-100% | 75-100% | 75-100% | 75-100% | 0-25% | 75-100% |
| 1042 | 75-100% | 75-100% | 75-100% | 75-100% | 0-25% | 75-100% |
| 1043 | 75-100% | 75-100% | 75-100% | 25-75% | 75-100% | 75-100% |
| 1044 | 0-25% | 0-25% | 0-25% | 0-25% | 0-25% | 0-25% |
| 1045 | 25-75% | 25-75% | 25-75% | 25-75% | 25-75% | 25-75% |
| 1046 | 75-100% | 75-100% | 75-100% | 75-100% | 75-100% | 25-75% |
| 1047 | 25-75% | 25-75% | 25-75% | 25-75% | 25-75% | 25-75% |
| 1048 | 75-100% | 75-100% | 75-100% | 75-100% | 0-25% | 75-100% |
| 1049 | 75-100% | 75-100% | 75-100% | 75-100% | 75-100% | 75-100% |
| 1050 | 0-25% | 0-25% | 0-25% | 0-25% | 0-25% | 75-100% |
| 1051 | 75-100% | 75-100% | 75-100% | 75-100% | 75-100% | 75-100% |
| 1052 | 75-100% | 75-100% | 75-100% | 25-75% | 75-100% | 75-100% |
| 1053 | 75-100% | 75-100% | 75-100% | 25-75% | 25-75% | 25-75% |
| 1054 | 0-25% | 0-25% | 0-25% | 0-25% | 0-25% | 0-25% |
| 1055 | 75-100% | 75-100% | 75-100% | 25-75% | 75-100% | 75-100% |
| 1056 | 75-100% | 75-100% | 75-100% | 75-100% | 75-100% | 75-100% |
| 1057 | 75-100% | 75-100% | 25-75% | 25-75% | 0-25% | 75-100% |
| 1058 | 75-100% | 75-100% | 75-100% | 75-100% | 75-100% | 75-100% |
| 1059 | 75-100% | 75-100% | 75-100% | 75-100% | 75-100% | 75-100% |
| 1060 | 25-75% | 25-75% | 25-75% | 25-75% | 25-75% | 25-75% |
| 1061 | 75-100% | 75-100% | 75-100% | 75-100% | 25-75% | 75-100% |
| 1062 | 75-100% | 75-100% | 75-100% | 75-100% | 0-25% | 75-100% |
| 1063 | 75-100% | 75-100% | 25-75% | 25-75% | 25-75% | 25-75% |
| 1064 | 0-25% | 0-25% | 0-25% | 0-25% | 0-25% | 0-25% |
| 1065 | 75-100% | 25-75% | 25-75% | 25-75% | 25-75% | 25-75% |
| 1066 | 75-100% | 75-100% | 75-100% | 75-100% | 75-100% | 75-100% |
| 1067 | 75-100% | 75-100% | 75-100% | 75-100% | 25-75% | 75-100% |
| 1068 | 25-75% | 0-25% | 0-25% | 0-25% | 0-25% | 0-25% |
| 1069 | 75-100% | 75-100% | 75-100% | 75-100% | 25-75% | 75-100% |
| 1070 | 25-75% | 25-75% | 75-100% | 75-100% | 0-25% | 75-100% |
| 1071 | 75-100% | 75-100% | 75-100% | 75-100% | 25-75% | 75-100% |
| 1072 | 75-100% | 75-100% | 75-100% | 75-100% | 0-25% | 75-100% |
| 1073 | 75-100% | 75-100% | 75-100% | 75-100% | 0-25% | 75-100% |
| 1074 | 75-100% | 75-100% | 75-100% | 75-100% | 0-25% | 75-100% |
| 1075 | 75-100% | 75-100% | 75-100% | 75-100% | 75-100% | 75-100% |
| 1076 | 75-100% | 75-100% | 75-100% | 75-100% | 0-25% | 75-100% |
| 1077 | 75-100% | 75-100% | 75-100% | 75-100% | 25-75% | 75-100% |
| 1078 | 75-100% | 75-100% | 75-100% | 25-75% | 0-25% | 75-100% |
| 1079 | 25-75% | 25-75% | 75-100% | 75-100% | 0-25% | 75-100% |
| 1080 | 75-100% | 75-100% | 75-100% | 75-100% | 75-100% | 75-100% |
| 1081 | 75-100% | 75-100% | 75-100% | 25-75% | 25-75% | 25-75% |
| 1082 | 75-100% | 75-100% | 25-75% | 25-75% | 25-75% | 25-75% |
| 1083 | 25-75% | 75-100% | 75-100% | 75-100% | 75-100% | 75-100% |
| 1084 | 75-100% | 75-100% | 75-100% | 75-100% | 0-25% | 75-100% |
| 1085 | 75-100% | 75-100% | 75-100% | 25-75% | 75-100% | 75-100% |
| 1086 | 75-100% | 75-100% | 75-100% | 75-100% | 0-25% | 75-100% |
| 1087 | 75-100% | 75-100% | 75-100% | 75-100% | 75-100% | 75-100% |
| 1088 | 75-100% | 75-100% | 75-100% | 75-100% | 75-100% | 75-100% |
| 1089 | 75-100% | 75-100% | 75-100% | 25-75% | 75-100% | 75-100% |
| 1090 | 75-100% | 75-100% | 25-75% | 25-75% | 25-75% | 25-75% |
| 1091 | 75-100% | 75-100% | 75-100% | 75-100% | 25-75% | 75-100% |
| 1092 | 75-100% | 75-100% | 75-100% | 75-100% | 75-100% | 75-100% |
| 1093 | 75-100% | 75-100% | 75-100% | 75-100% | 75-100% | 75-100% |
| 1094 | 75-100% | 75-100% | 75-100% | 75-100% | 75-100% | 75-100% |
| 1095 | 75-100% | 75-100% | 75-100% | 75-100% | 75-100% | 75-100% |
| 1096 | 75-100% | 75-100% | 75-100% | ND | 25-75% | 25-75% |
| 1097 | 75-100% | 75-100% | 75-100% | 75-100% | 75-100% | 75-100% |
| 1098 | 75-100% | 75-100% | 75-100% | 75-100% | 75-100% | 75-100% |
| 1099 | 75-100% | 75-100% | 75-100% | 75-100% | 75-100% | 75-100% |
| 1100 | 75-100% | 75-100% | 25-75% | 25-75% | 0-25% | 0-25% |
| 1101 | 75-100% | 75-100% | 75-100% | 75-100% | 75-100% | 75-100% |
| 1102 | 75-100% | 75-100% | 75-100% | 75-100% | 75-100% | 75-100% |
| 1103 | 75-100% | 75-100% | 75-100% | 75-100% | 75-100% | 75-100% |
| 1104 | 75-100% | 75-100% | 25-75% | 25-75% | 25-75% | 25-75% |
| 1105 | 75-100% | 75-100% | 75-100% | 75-100% | 75-100% | 75-100% |
| 1106 | 75-100% | 75-100% | 25-75% | 25-75% | 25-75% | 25-75% |
| 1107 | 75-100% | 75-100% | 0-25% | 0-25% | 0-25% | 0-25% |
| 1108 | 0-25% | 0-25% | 0-25% | 0-25% | 0-25% | 0-25% |
| 1109 | 75-100% | 75-100% | 75-100% | 25-75% | 75-100% | 75-100% |
| 1110 | 0-25% | 0-25% | 0-25% | 75-100% | 75-100% | 75-100% |
| 1111 | 25-75% | 25-75% | 25-75% | 25-75% | 25-75% | 25-75% |
| 1112 | 0-25% | 0-25% | 0-25% | 0-25% | 0-25% | 0-25% |
| 1113 | 75-100% | 75-100% | 0-25% | 0-25% | 0-25% | 25-75% |
| 1114 | 0-25% | 0-25% | 75-100% | 75-100% | 75-100% | 75-100% |
| 1115 | 0-25% | 0-25% | 0-25% | 0-25% | 0-25% | 0-25% |
| 1116 | 75-100% | 75-100% | 25-75% | 25-75% | 25-75% | 25-75% |
| 1117 | 75-100% | 75-100% | 75-100% | 75-100% | 75-100% | 75-100% |
| 1118 | 25-75% | 25-75% | 25-75% | 25-75% | 25-75% | 25-75% |
| 1119 | 25-75% | 25-75% | 25-75% | 25-75% | 0-25% | 25-75% |
| 1120 | 75-100% | 75-100% | 75-100% | 75-100% | 75-100% | 75-100% |
| 1121 | 75-100% | 75-100% | 75-100% | 25-75% | 0-25% | 75-100% |
| 1122 | 75-100% | 75-100% | 0-25% | 0-25% | 0-25% | 0-25% |
| 1123 | 75-100% | 75-100% | 75-100% | 75-100% | 75-100% | 75-100% |
| 1124 | 75-100% | 75-100% | 75-100% | 75-100% | 75-100% | 75-100% |
| 1125 | 75-100% | 75-100% | 75-100% | 75-100% | 75-100% | 75-100% |
| 1126 | 0-25% | 0-25% | 25-75% | 75-100% | 75-100% | 75-100% |
| 1127 | 25-75% | 25-75% | 25-75% | 25-75% | 25-75% | 0-25% |
| 1128 | 0-25% | 0-25% | 75-100% | 75-100% | 75-100% | 75-100% |
| 1129 | 75-100% | 75-100% | 75-100% | 75-100% | 75-100% | 25-75% |
| 1130 | 75-100% | 75-100% | ND | ND | 25-75% | 75-100% |
| 1131 | 0-25% | 0-25% | 0-25% | 0-25% | 0-25% | 75-100% |
| 1132 | 0-25% | 0-25% | 75-100% | 75-100% | 75-100% | 75-100% |
| 1133 | 75-100% | 75-100% | 75-100% | 75-100% | 75-100% | 75-100% |
| 1134 | 25-75% | 25-75% | 75-100% | 75-100% | 75-100% | 75-100% |
| 1135 | 75-100% | 75-100% | 25-75% | 25-75% | 25-75% | 25-75% |
| 1136 | 75-100% | 75-100% | 75-100% | 75-100% | 75-100% | 75-100% |
| 1137 | 25-75% | 75-100% | 25-75% | 25-75% | 25-75% | 25-75% |
| 1138 | 75-100% | 75-100% | 75-100% | 75-100% | 75-100% | 75-100% |
| 1139 | 75-100% | 75-100% | 75-100% | 75-100% | 0-25% | 75-100% |
| 1140 | 75-100% | 75-100% | 75-100% | 75-100% | 0-25% | 75-100% |
| 1141 | 0-25% | 0-25% | 25-75% | 25-75% | 75-100% | 75-100% |
| 1142 | 75-100% | 75-100% | 75-100% | 75-100% | 75-100% | 75-100% |
| 1143 | 75-100% | 25-75% | 25-75% | 75-100% | 75-100% | 75-100% |

| SEQ ID | | | | | Skeletal | |
|---|---|---|---|---|---|---|
| NO: Genomic | Keratinocyte | Liver | Melanocyte | Placenta | Muscle | Sperm |
| 844 | 75-100% | 75-100% | 75-100% | 75-100% | 75-100% | 0-25% |
| 845 | 75-100% | 75-100% | 75-100% | 75-100% | 75-100% | 0-25% |
| 846 | 75-100% | 25-75% | 75-100% | 75-100% | 75-100% | 75-100% |
| 847 | 0-25% | 0-25% | 0-25% | 0-25% | 0-25% | 0-25% |
| 848 | 75-100% | 75-100% | 75-100% | 75-100% | 75-100% | 75-100% |
| 849 | 75-100% | 75-100% | 0-25% | 75-100% | 75-100% | 75-100% |
| 850 | 75-100% | 75-100% | 75-100% | 0-25% | 75-100% | 0-25% |
| 851 | 75-100% | 75-100% | 75-100% | 75-100% | 0-25% | 75-100% |
| 852 | 75-100% | 75-100% | 75-100% | 75-100% | 75-100% | 0-25% |
| 853 | 75-100% | 25-75% | 75-100% | 25-75% | 25-75% | 0-25% |
| 854 | 0-25% | 0-25% | 75-100% | 0-25% | 0-25% | 75-100% |
| 855 | 75-100% | 75-100% | 75-100% | 25-75% | 25-75% | 75-100% |
| 856 | 25-75% | 75-100% | 75-100% | 25-75% | 25-75% | 75-100% |
| 857 | 0-25% | 0-25% | 0-25% | 0-25% | 0-25% | 0-25% |
| 858 | 75-100% | 25-75% | 75-100% | 75-100% | 75-100% | 0-25% |
| 859 | 75-100% | 75-100% | 75-100% | 75-100% | 75-100% | 0-25% |
| 860 | 25-75% | 0-25% | 0-25% | 75-100% | 75-100% | 0-25% |
| 861 | 75-100% | 75-100% | 75-100% | 75-100% | 75-100% | 75-100% |
| 862 | 75-100% | 75-100% | 75-100% | 75-100% | 75-100% | 0-25% |
| 863 | 75-100% | 75-100% | 75-100% | 75-100% | 25-75% | 75-100% |
| 864 | 25-75% | 75-100% | 75-100% | 75-100% | 75-100% | 75-100% |
| 865 | 75-100% | 25-75% | 75-100% | 75-100% | 75-100% | 0-25% |
| 866 | 25-75% | 75-100% | 75-100% | 75-100% | 75-100% | 0-25% |
| 867 | 75-100% | 75-100% | 75-100% | 25-75% | 75-100% | 75-100% |
| 868 | 75-100% | 75-100% | 75-100% | 75-100% | 75-100% | 0-25% |
| 869 | 25-75% | 25-75% | 25-75% | 25-75% | 25-75% | 0-25% |
| 870 | 75-100% | 25-75% | 25-75% | 25-75% | 25-75% | 25-75% |
| 871 | 75-100% | 75-100% | 75-100% | 75-100% | 25-75% | 75-100% |
| 872 | 75-100% | 75-100% | ND | 75-100% | 25-75% | 75-100% |
| 873 | 25-75% | 25-75% | 25-75% | 25-75% | 25-75% | 75-100% |
| 874 | 75-100% | 75-100% | 0-25% | 75-100% | 75-100% | 75-100% |
| 875 | 75-100% | 75-100% | 75-100% | 75-100% | 75-100% | 75-100% |
| 876 | 0-25% | 0-25% | 0-25% | 0-25% | 0-25% | 0-25% |
| 877 | 75-100% | 75-100% | 75-100% | 75-100% | 75-100% | 75-100% |
| 878 | 0-25% | 75-100% | 75-100% | 75-100% | 75-100% | 75-100% |
| 879 | 25-75% | 75-100% | 25-75% | 25-75% | 25-75% | 75-100% |
| 880 | 75-100% | 75-100% | 75-100% | 75-100% | 75-100% | 75-100% |
| 881 | 0-25% | 25-75% | 75-100% | 25-75% | 25-75% | ND |
| 882 | 75-100% | 75-100% | 75-100% | 75-100% | 75-100% | 75-100% |
| 883 | 75-100% | 75-100% | 75-100% | 75-100% | 75-100% | 75-100% |
| 884 | 75-100% | 75-100% | 75-100% | 75-100% | 0-25% | 0-25% |
| 885 | 0-25% | 75-100% | 75-100% | 75-100% | 25-75% | 75-100% |
| 886 | 75-100% | 75-100% | 0-25% | 75-100% | 75-100% | 75-100% |
| 887 | 75-100% | 25-75% | 75-100% | 75-100% | 75-100% | 75-100% |
| 888 | 75-100% | 25-75% | 75-100% | 75-100% | 75-100% | 75-100% |
| 889 | 75-100% | 75-100% | 75-100% | 75-100% | 75-100% | 75-100% |
| 890 | 75-100% | 75-100% | 75-100% | 75-100% | 75-100% | 75-100% |
| 891 | 0-25% | 75-100% | 75-100% | 75-100% | 75-100% | 75-100% |
| 892 | 0-25% | 75-100% | 75-100% | 75-100% | 75-100% | 75-100% |
| 893 | 0-25% | 75-100% | 75-100% | 75-100% | 25-75% | 75-100% |
| 894 | 0-25% | 25-75% | 25-75% | 25-75% | 25-75% | 75-100% |
| 895 | 75-100% | 75-100% | 75-100% | 75-100% | 75-100% | 0-25% |
| 896 | 0-25% | 0-25% | 0-25% | 0-25% | 0-25% | 75-100% |
| 897 | 75-100% | 75-100% | 75-100% | 75-100% | 25-75% | 75-100% |
| 898 | 25-75% | 25-75% | 25-75% | 25-75% | 25-75% | 75-100% |
| 899 | 0-25% | 25-75% | 25-75% | 25-75% | 25-75% | 75-100% |
| 900 | 75-100% | 75-100% | 75-100% | 75-100% | 25-75% | 75-100% |
| 901 | 0-25% | 0-25% | 0-25% | 0-25% | 0-25% | 75-100% |
| 902 | 75-100% | 75-100% | 25-75% | 75-100% | 75-100% | 75-100% |
| 903 | 75-100% | 75-100% | 75-100% | 75-100% | 25-75% | 75-100% |
| 904 | 75-100% | 75-100% | 75-100% | 75-100% | 75-100% | 75-100% |
| 905 | 75-100% | 75-100% | 75-100% | 75-100% | 75-100% | 75-100% |
| 906 | 75-100% | 75-100% | 75-100% | 75-100% | 75-100% | 75-100% |
| 907 | 75-100% | 75-100% | 75-100% | 75-100% | 75-100% | 75-100% |
| 908 | 0-25% | 25-75% | 0-25% | 25-75% | 25-75% | 75-100% |
| 909 | 25-75% | 25-75% | 0-25% | 25-75% | 25-75% | 75-100% |
| 910 | 0-25% | 25-75% | 0-25% | 0-25% | 75-100% | 75-100% |
| 911 | 75-100% | 75-100% | 75-100% | 75-100% | 75-100% | 75-100% |
| 912 | 25-75% | 25-75% | 25-75% | 25-75% | 25-75% | 75-100% |
| 913 | 25-75% | 25-75% | 25-75% | 25-75% | 25-75% | 75-100% |
| 914 | 25-75% | 25-75% | 25-75% | 25-75% | 25-75% | 75-100% |
| 915 | 0-25% | 25-75% | 0-25% | 0-25% | 0-25% | 75-100% |
| 916 | 0-25% | 0-25% | 0-25% | 0-25% | 0-25% | 75-100% |
| 917 | 0-25% | 0-25% | 0-25% | 0-25% | 0-25% | 75-100% |
| 918 | 75-100% | 75-100% | 0-25% | 25-75% | 75-100% | 75-100% |
| 919 | 75-100% | 75-100% | 0-25% | 25-75% | 25-75% | 75-100% |
| 920 | 25-75% | 25-75% | 25-75% | 25-75% | 25-75% | 75-100% |
| 921 | 75-100% | 75-100% | 75-100% | 75-100% | 75-100% | 75-100% |
| 922 | 0-25% | 0-25% | 0-25% | 0-25% | 0-25% | 75-100% |
| 923 | 0-25% | 0-25% | 0-25% | 0-25% | 0-25% | 75-100% |
| 924 | 75-100% | 75-100% | 75-100% | 75-100% | 25-75% | 75-100% |
| 925 | 0-25% | 75-100% | 75-100% | 75-100% | 75-100% | 0-25% |
| 926 | 0-25% | 75-100% | 75-100% | 75-100% | 75-100% | 75-100% |
| 927 | 25-75% | 25-75% | 0-25% | 25-75% | 25-75% | 0-25% |
| 928 | 75-100% | 75-100% | 75-100% | 75-100% | 75-100% | 75-100% |
| 929 | 75-100% | 75-100% | 75-100% | 75-100% | 0-25% | 75-100% |
| 930 | 0-25% | 75-100% | 75-100% | 75-100% | 75-100% | 75-100% |
| 931 | 75-100% | 75-100% | 75-100% | 75-100% | 25-75% | 75-100% |
| 932 | 75-100% | 25-75% | 25-75% | 25-75% | 25-75% | 0-25% |
| 933 | 25-75% | 25-75% | 25-75% | 25-75% | 75-100% | 0-25% |
| 934 | 75-100% | 25-75% | 75-100% | 75-100% | 75-100% | 75-100% |
| 935 | 75-100% | 75-100% | 75-100% | 75-100% | 75-100% | 75-100% |
| 936 | 0-25% | 75-100% | 0-25% | 0-25% | 0-25% | ND |
| 937 | 0-25% | 75-100% | 75-100% | 75-100% | 75-100% | 75-100% |
| 938 | 75-100% | 75-100% | 75-100% | 75-100% | 75-100% | 0-25% |
| 939 | 0-25% | 75-100% | 0-25% | 0-25% | 0-25% | ND |
| 940 | 75-100% | 75-100% | 75-100% | 75-100% | 75-100% | 0-25% |
| 941 | 75-100% | 75-100% | 75-100% | 75-100% | 75-100% | 0-25% |
| 942 | 75-100% | 75-100% | 75-100% | 75-100% | 75-100% | 75-100% |
| 943 | 0-25% | 75-100% | 75-100% | 25-75% | 25-75% | 75-100% |
| 944 | 75-100% | 75-100% | 75-100% | 75-100% | 75-100% | 75-100% |
| 945 | 75-100% | 75-100% | 75-100% | 75-100% | 25-75% | 75-100% |
| 946 | 75-100% | 75-100% | 75-100% | 75-100% | 75-100% | 75-100% |
| 947 | 0-25% | 75-100% | 75-100% | 75-100% | 75-100% | 75-100% |
| 948 | 75-100% | 75-100% | 75-100% | 75-100% | 25-75% | 75-100% |
| 949 | 25-75% | 25-75% | 25-75% | 25-75% | 25-75% | 25-75% |
| 950 | 25-75% | 0-25% | 25-75% | 25-75% | 75-100% | 0-25% |
| 951 | 0-25% | 75-100% | 75-100% | 75-100% | 75-100% | 75-100% |
| 952 | 0-25% | 75-100% | 75-100% | 75-100% | 75-100% | 0-25% |
| 953 | 75-100% | 75-100% | 75-100% | 75-100% | 75-100% | 0-25% |
| 954 | 75-100% | 75-100% | 25-75% | 75-100% | 75-100% | 75-100% |
| 955 | 75-100% | 75-100% | 75-100% | 75-100% | 25-75% | 75-100% |
| 956 | 0-25% | 0-25% | 75-100% | 75-100% | 75-100% | 75-100% |
| 957 | 0-25% | 75-100% | 75-100% | 75-100% | 75-100% | 75-100% |
| 958 | 0-25% | 0-25% | 0-25% | 0-25% | 0-25% | 75-100% |
| 959 | 75-100% | 75-100% | 75-100% | 75-100% | 75-100% | 75-100% |
| 960 | 75-100% | 25-75% | 75-100% | 75-100% | 75-100% | 75-100% |
| 961 | 75-100% | 75-100% | 75-100% | 75-100% | 25-75% | 75-100% |
| 962 | 25-75% | 25-75% | 25-75% | 25-75% | 25-75% | 75-100% |
| 963 | 0-25% | 75-100% | 75-100% | 75-100% | 75-100% | 0-25% |
| 964 | 0-25% | 75-100% | 75-100% | 75-100% | 75-100% | 75-100% |
| 965 | 75-100% | 75-100% | 75-100% | 75-100% | 75-100% | 75-100% |
| 966 | 75-100% | 75-100% | ND | 75-100% | 75-100% | 75-100% |
| 967 | 0-25% | 75-100% | 75-100% | 75-100% | 75-100% | 75-100% |
| 968 | 0-25% | 75-100% | 0-25% | 0-25% | 0-25% | 75-100% |
| 969 | 0-25% | 0-25% | 0-25% | 25-75% | 0-25% | 0-25% |
| 970 | 0-25% | 75-100% | 75-100% | 75-100% | 75-100% | 75-100% |
| 971 | 75-100% | 0-25% | 75-100% | 75-100% | 75-100% | 75-100% |
| 972 | 0-25% | 25-75% | 25-75% | 25-75% | 25-75% | 0-25% |
| 973 | 0-25% | 25-75% | 25-75% | 25-75% | 25-75% | 0-25% |
| 974 | 75-100% | 75-100% | 75-100% | 75-100% | 75-100% | 0-25% |
| 975 | 75-100% | 75-100% | 75-100% | 75-100% | 25-75% | 75-100% |
| 976 | 0-25% | 0-25% | 25-75% | ND | 0-25% | 0-25% |
| 977 | 0-25% | 75-100% | 25-75% | 0-25% | 0-25% | 75-100% |
| 978 | 75-100% | 75-100% | 75-100% | 75-100% | 25-75% | 75-100% |
| 979 | 75-100% | 25-75% | 75-100% | 75-100% | 75-100% | 75-100% |
| 980 | 25-75% | 75-100% | 75-100% | 75-100% | 75-100% | 75-100% |
| 981 | 0-25% | 75-100% | 75-100% | 75-100% | 75-100% | 75-100% |
| 982 | 0-25% | 75-100% | 75-100% | 75-100% | 75-100% | 75-100% |
| 983 | 75-100% | 75-100% | 75-100% | 75-100% | 75-100% | 75-100% |
| 984 | 75-100% | 25-75% | 75-100% | 75-100% | 75-100% | |
| 985 | 75-100% | 25-75% | 75-100% | 75-100% | 75-100% | 75-100% |
| 986 | 0-25% | 0-25% | 0-25% | 0-25% | 0-25% | 0-25% |
| 987 | 0-25% | 25-75% | 75-100% | 75-100% | 75-100% | 75-100% |
| 988 | 25-75% | 25-75% | 25-75% | 25-75% | 25-75% | 75-100% |
| 989 | 0-25% | 0-25% | 0-25% | 0-25% | 0-25% | 75-100% |
| 990 | 0-25% | 75-100% | 75-100% | 75-100% | 75-100% | 75-100% |
| 991 | 75-100% | 75-100% | 75-100% | 75-100% | 75-100% | 0-25% |
| 992 | 0-25% | 25-75% | 25-75% | 25-75% | 25-75% | 75-100% |
| 993 | 75-100% | 75-100% | 75-100% | 75-100% | 75-100% | 75-100% |
| 994 | 75-100% | 75-100% | 75-100% | 75-100% | 75-100% | 75-100% |
| 995 | 0-25% | 75-100% | 75-100% | 75-100% | 25-75% | 75-100% |
| 996 | 0-25% | 75-100% | 75-100% | 75-100% | 75-100% | 0-25% |
| 997 | 0-25% | 25-75% | 25-75% | 25-75% | 25-75% | ND |
| 998 | 75-100% | 75-100% | 75-100% | 75-100% | 75-100% | 75-100% |
| 999 | 0-25% | 25-75% | 0-25% | 0-25% | 0-25% | 0-25% |
| 1000 | 75-100% | 75-100% | 75-100% | 75-100% | 25-75% | 75-100% |
| 1001 | 0-25% | 75-100% | 75-100% | 75-100% | 75-100% | 0-25% |
| 1002 | 0-25% | 25-75% | 25-75% | 25-75% | 25-75% | 75-100% |
| 1003 | 75-100% | 25-75% | 75-100% | 25-75% | 25-75% | 75-100% |
| 1004 | 75-100% | 75-100% | 75-100% | 75-100% | 25-75% | 0-25% |
| 1005 | 75-100% | 75-100% | 75-100% | 75-100% | 75-100% | 75-100% |
| 1006 | 0-25% | 75-100% | 75-100% | 75-100% | 75-100% | 75-100% |
| 1007 | 75-100% | 0-25% | 75-100% | 0-25% | 0-25% | 0-25% |
| 1008 | 75-100% | 75-100% | 75-100% | 75-100% | 25-75% | 75-100% |
| 1009 | 75-100% | 75-100% | 75-100% | 75-100% | 75-100% | 0-25% |
| 1010 | 0-25% | 0-25% | 0-25% | 0-25% | 0-25% | 0-25% |
| 1011 | 75-100% | 75-100% | 75-100% | 75-100% | 25-75% | 0-25% |
| 1012 | 75-100% | 75-100% | 75-100% | 75-100% | 75-100% | 0-25% |
| 1013 | 75-100% | 25-75% | 75-100% | 75-100% | 75-100% | 75-100% |
| 1014 | 75-100% | 25-75% | 75-100% | 75-100% | 75-100% | 75-100% |
| 1015 | 0-25% | 0-25% | 0-25% | 0-25% | 25-75% | 75-100% |
| 1016 | 75-100% | 75-100% | 75-100% | 75-100% | 25-75% | 75-100% |
| 1017 | 75-100% | 75-100% | 75-100% | 75-100% | 75-100% | 75-100% |
| 1018 | 0-25% | 75-100% | 75-100% | 75-100% | 75-100% | 75-100% |
| 1019 | 0-25% | 25-75% | 0-25% | 0-25% | 0-25% | 0-25% |
| 1020 | 75-100% | 0-25% | 0-25% | 0-25% | 0-25% | 0-25% |
| 1021 | 0-25% | 75-100% | 75-100% | 0-25% | 0-25% | 0-25% |
| 1022 | 0-25% | 75-100% | 0-25% | 0-25% | 0-25% | 0-25% |
| 1023 | 0-25% | 75-100% | 75-100% | 75-100% | 25-75% | 75-100% |
| 1024 | 25-75% | 25-75% | 25-75% | 0-25% | 0-25% | 0-25% |
| 1025 | 75-100% | 75-100% | 75-100% | 75-100% | 25-75% | 75-100% |
| 1026 | 75-100% | 75-100% | 75-100% | 75-100% | 75-100% | 75-100% |
| 1027 | 75-100% | 75-100% | 0-25% | 25-75% | 75-100% | 75-100% |
| 1028 | 0-25% | 0-25% | 0-25% | 0-25% | 0-25% | 75-100% |
| 1029 | 75-100% | 75-100% | 75-100% | 75-100% | 75-100% | 75-100% |
| 1030 | 75-100% | 25-75% | 75-100% | 75-100% | 25-75% | 75-100% |
| 1031 | 0-25% | 75-100% | 75-100% | 75-100% | 25-75% | 75-100% |
| 1032 | 75-100% | 75-100% | 75-100% | 75-100% | 75-100% | 75-100% |
| 1033 | 0-25% | 75-100% | 75-100% | 75-100% | 75-100% | 75-100% |
| 1034 | 0-25% | 75-100% | 75-100% | 75-100% | 75-100% | 75-100% |
| 1035 | 25-75% | 75-100% | 75-100% | 75-100% | 75-100% | 75-100% |
| 1036 | 25-75% | 75-100% | 75-100% | 75-100% | 75-100% | 75-100% |
| 1037 | 75-100% | 25-75% | 75-100% | 75-100% | 75-100% | 75-100% |
| 1038 | 75-100% | 75-100% | 75-100% | 75-100% | 75-100% | 75-100% |
| 1039 | 0-25% | 75-100% | 75-100% | 75-100% | 75-100% | 75-100% |
| 1040 | 75-100% | 75-100% | 0-25% | 0-25% | 75-100% | 75-100% |
| 1041 | 75-100% | 75-100% | 75-100% | 75-100% | 75-100% | 75-100% |
| 1042 | 75-100% | 75-100% | 75-100% | 75-100% | 75-100% | 75-100% |
| 1043 | 75-100% | 75-100% | 75-100% | 75-100% | 25-75% | 75-100% |
| 1044 | 75-100% | 75-100% | 0-25% | 0-25% | 0-25% | ND |
| 1045 | 75-100% | 25-75% | 25-75% | 25-75% | 25-75% | 75-100% |
| 1046 | 75-100% | 75-100% | 75-100% | 75-100% | 75-100% | 75-100% |
| 1047 | 25-75% | 75-100% | 25-75% | 25-75% | 25-75% | 75-100% |
| 1048 | 75-100% | 75-100% | 75-100% | 75-100% | 25-75% | 75-100% |
| 1049 | 75-100% | 75-100% | 0-25% | 75-100% | 75-100% | 75-100% |
| 1050 | 0-25% | 0-25% | 0-25% | 0-25% | 0-25% | 75-100% |
| 1051 | 75-100% | 25-75% | 75-100% | 75-100% | 75-100% | 75-100% |
| 1052 | 75-100% | 75-100% | 75-100% | 75-100% | 25-75% | 75-100% |
| 1053 | 25-75% | 75-100% | 75-100% | 75-100% | 0-25% | 75-100% |
| 1054 | 0-25% | 0-25% | 0-25% | 0-25% | 0-25% | 75-100% |
| 1055 | 75-100% | 75-100% | 75-100% | 75-100% | 25-75% | 75-100% |
| 1056 | 0-25% | 75-100% | 75-100% | 75-100% | 75-100% | 75-100% |
| 1057 | 75-100% | 75-100% | 75-100% | 25-75% | 25-75% | 0-25% |
| 1058 | 0-25% | 75-100% | 75-100% | 75-100% | 25-75% | 75-100% |
| 1059 | 0-25% | 75-100% | 75-100% | 25-75% | 25-75% | 75-100% |
| 1060 | 25-75% | 25-75% | 25-75% | ND | 75-100% | 75-100% |
| 1061 | 75-100% | 75-100% | 75-100% | 75-100% | 75-100% | 75-100% |
| 1062 | 75-100% | 75-100% | 75-100% | 75-100% | 25-75% | 75-100% |
| 1063 | 25-75% | 25-75% | 25-75% | 25-75% | 25-75% | 75-100% |
| 1064 | 75-100% | 0-25% | 0-25% | 0-25% | 0-25% | 75-100% |
| 1065 | 25-75% | 25-75% | 25-75% | 25-75% | 25-75% | 75-100% |
| 1066 | 0-25% | 75-100% | 75-100% | 75-100% | 75-100% | 75-100% |
| 1067 | 75-100% | 75-100% | 75-100% | 75-100% | 75-100% | 75-100% |
| 1068 | 0-25% | 0-25% | 0-25% | 0-25% | 0-25% | 75-100% |
| 1069 | 25-75% | 75-100% | 75-100% | 75-100% | 75-100% | 75-100% |
| 1070 | 0-25% | 75-100% | 75-100% | 75-100% | 75-100% | 75-100% |
| 1071 | 25-75% | 75-100% | 25-75% | 75-100% | 75-100% | 75-100% |
| 1072 | 75-100% | 75-100% | 75-100% | 75-100% | 75-100% | 0-25% |
| 1073 | 75-100% | 75-100% | 75-100% | 75-100% | 75-100% | 75-100% |
| 1074 | 75-100% | 75-100% | 75-100% | 75-100% | 75-100% | 75-100% |
| 1075 | 75-100% | 75-100% | 0-25% | 25-75% | 25-75% | 75-100% |
| 1076 | 75-100% | 75-100% | 75-100% | 75-100% | 75-100% | 75-100% |
| 1077 | 75-100% | 75-100% | 75-100% | 75-100% | 75-100% | 75-100% |
| 1078 | 75-100% | 75-100% | 75-100% | 75-100% | 25-75% | 75-100% |
| 1079 | 75-100% | 75-100% | 75-100% | 75-100% | 75-100% | 75-100% |
| 1080 | 0-25% | 75-100% | 75-100% | 75-100% | 75-100% | |
| 1081 | 0-25% | 75-100% | 75-100% | 75-100% | 25-75% | 75-100% |
| 1082 | 25-75% | 25-75% | 25-75% | 25-75% | 25-75% | 75-100% |
| 1083 | 75-100% | 75-100% | 75-100% | 75-100% | 75-100% | 75-100% |
| 1084 | 75-100% | 75-100% | 75-100% | 75-100% | 75-100% | 75-100% |
| 1085 | 75-100% | 75-100% | 75-100% | 75-100% | 75-100% | 75-100% |
| 1086 | 75-100% | 75-100% | 75-100% | 75-100% | 25-75% | 75-100% |
| 1087 | 75-100% | 75-100% | 0-25% | 75-100% | 75-100% | 75-100% |
| 1088 | 75-100% | 75-100% | 75-100% | 75-100% | 25-75% | 75-100% |
| 1089 | 75-100% | 75-100% | 75-100% | ND | 25-75% | ND |
| 1090 | 25-75% | 25-75% | 25-75% | 25-75% | 25-75% | 75-100% |
| 1091 | 75-100% | 75-100% | 75-100% | 75-100% | 75-100% | 75-100% |
| 1092 | 75-100% | 25-75% | 75-100% | 75-100% | 75-100% | 75-100% |
| 1093 | 0-25% | 75-100% | 75-100% | 75-100% | 75-100% | 75-100% |
| 1094 | 0-25% | 75-100% | 75-100% | 75-100% | 75-100% | 75-100% |
| 1095 | 75-100% | 75-100% | 75-100% | 75-100% | 0-25% | ND |
| 1096 | 25-75% | 25-75% | 25-75% | 25-75% | 25-75% | 0-25% |
| 1097 | 0-25% | 75-100% | 75-100% | 75-100% | 75-100% | 75-100% |
| 1098 | 0-25% | 75-100% | 75-100% | 75-100% | 75-100% | 75-100% |
| 1099 | 75-100% | 75-100% | 75-100% | 75-100% | 25-75% | 75-100% |
| 1100 | 0-25% | 25-75% | 0-25% | 0-25% | 25-75% | 0-25% |
| 1101 | 0-25% | 75-100% | 75-100% | 75-100% | 75-100% | 75-100% |
| 1102 | 75-100% | 75-100% | 0-25% | 75-100% | 75-100% | 75-100% |
| 1103 | 75-100% | 75-100% | 0-25% | 75-100% | 75-100% | 75-100% |
| 1104 | 25-75% | 75-100% | 25-75% | 25-75% | 0-25% | ND |
| 1105 | 75-100% | 75-100% | 25-75% | 75-100% | 75-100% | 75-100% |
| 1106 | 25-75% | 25-75% | 25-75% | 25-75% | 25-75% | 75-100% |
| 1107 | 0-25% | 0-25% | 0-25% | 0-25% | 0-25% | 75-100% |
| 1108 | 75-100% | 0-25% | 0-25% | 0-25% | 0-25% | ND |
| 1109 | 75-100% | 75-100% | 75-100% | 75-100% | 25-75% | 75-100% |
| 1110 | 75-100% | 75-100% | 75-100% | 75-100% | 75-100% | 75-100% |
| 1111 | 25-75% | 75-100% | 25-75% | 25-75% | 25-75% | 25-75% |
| 1112 | 0-25% | 0-25% | 75-100% | 0-25% | 0-25% | 75-100% |
| 1113 | 0-25% | 25-75% | 0-25% | 0-25% | 25-75% | 75-100% |
| 1114 | 75-100% | 75-100% | 75-100% | 75-100% | 75-100% | 75-100% |
| 1115 | 0-25% | 25-75% | 0-25% | 0-25% | 0-25% | 0-25% |
| 1116 | 25-75% | 25-75% | 25-75% | 25-75% | 25-75% | 75-100% |
| 1117 | 75-100% | 75-100% | 75-100% | 75-100% | 25-75% | 75-100% |
| 1118 | 75-100% | 25-75% | 25-75% | 25-75% | 25-75% | 75-100% |
| 1119 | 25-75% | 25-75% | 25-75% | 25-75% | 25-75% | ND |
| 1120 | 0-25% | 75-100% | 75-100% | 75-100% | 75-100% | 0-25% |
| 1121 | 75-100% | 75-100% | 75-100% | 75-100% | 25-75% | 75-100% |
| 1122 | 0-25% | 0-25% | 0-25% | 0-25% | 0-25% | 0-25% |
| 1123 | 0-25% | 75-100% | 75-100% | 75-100% | 75-100% | 75-100% |
| 1124 | 75-100% | 25-75% | 75-100% | 75-100% | 75-100% | 75-100% |
| 1125 | 75-100% | 75-100% | 75-100% | 75-100% | 75-100% | 0-25% |
| 1126 | 75-100% | 25-75% | 75-100% | 75-100% | 75-100% | 75-100% |
| 1127 | 75-100% | 25-75% | 25-75% | 25-75% | 0-25% | 0-25% |
| 1128 | 75-100% | 75-100% | 75-100% | 75-100% | 75-100% | 75-100% |
| 1129 | 75-100% | 75-100% | 75-100% | 75-100% | 75-100% | 75-100% |
| 1130 | 75-100% | 75-100% | 75-100% | ND | 75-100% | 75-100% |
| 1131 | 0-25% | 25-75% | 0-25% | 0-25% | 25-75% | 75-100% |
| 1132 | 75-100% | 0-25% | 75-100% | 75-100% | 75-100% | 75-100% |
| 1133 | 0-25% | 75-100% | 75-100% | 75-100% | 75-100% | 75-100% |
| 1134 | 0-25% | 75-100% | 75-100% | 75-100% | 75-100% | 75-100% |
| 1135 | 25-75% | 25-75% | 25-75% | 25-75% | 75-100% | 75-100% |
| 1136 | 75-100% | 25-75% | 75-100% | 75-100% | 75-100% | 75-100% |
| 1137 | 25-75% | 75-100% | 25-75% | 25-75% | 25-75% | 75-100% |
| 1138 | 0-25% | 75-100% | 75-100% | 75-100% | 75-100% | 75-100% |
| 1139 | 75-100% | 75-100% | 75-100% | 75-100% | 75-100% | 75-100% |
| 1140 | 0-25% | 75-100% | 75-100% | 75-100% | 75-100% | 75-100% |
| 1141 | 75-100% | 75-100% | 75-100% | 75-100% | 75-100% | 75-100% |
| 1142 | 75-100% | 25-75% | 75-100% | 75-100% | 75-100% | 75-100% |
| 1143 | 25-75% | 75-100% | 75-100% | 75-100% | 75-100% | 75-100% |

TABLE 4
Preferred tissue markers according to the present invention

| SEQ ID | |
|---|---|
| NO: | |
| (Genomic) | Tissue |
| 942 | CD4 T-lymphocyte |
| 1065 | CD4 T-lymphocyte |
| 1068 | CD4 T-lymphocyte |
| 1083 | CD4 T-lymphocyte |
| 847 | CD4 T-lymphocyte, CD8 T-lymphocyte |
| 848 | CD4 T-lymphocyte, CD8 T-lymphocyte |
| 857 | CD4 T-lymphocyte, CD8 T-lymphocyte |
| 869 | CD4 T-lymphocyte, CD8 T-lymphocyte |
| 873 | CD4 T-lymphocyte, CD8 T-lymphocyte |
| 876 | CD4 T-lymphocyte, CD8 T-lymphocyte |
| 880 | CD4 T-lymphocyte, CD8 T-lymphocyte |
| 882 | CD4 T-lymphocyte, CD8 T-lymphocyte |
| 883 | CD4 T-lymphocyte, CD8 T-lymphocyte |
| 889 | CD4 T-lymphocyte, CD8 T-lymphocyte |
| 898 | CD4 T-lymphocyte, CD8 T-lymphocyte |
| 899 | CD4 T-lymphocyte, CD8 T-lymphocyte |
| 905 | CD4 T-lymphocyte, CD8 T-lymphocyte |
| 912 | CD4 T-lymphocyte, CD8 T-lymphocyte |
| 913 | CD4 T-lymphocyte, CD8 T-lymphocyte |
| 914 | CD4 T-lymphocyte, CD8 T-lymphocyte |
| 920 | CD4 T-lymphocyte, CD8 T-lymphocyte |
| 921 | CD4 T-lymphocyte, CD8 T-lymphocyte |
| 922 | CD4 T-lymphocyte, CD8 T-lymphocyte |
| 923 | CD4 T-lymphocyte, CD8 T-lymphocyte |
| 924 | CD4 T-lymphocyte, CD8 T-lymphocyte |
| 928 | CD4 T-lymphocyte, CD8 T-lymphocyte |
| 944 | CD4 T-lymphocyte, CD8 T-lymphocyte |
| 946 | CD4 T-lymphocyte, CD8 T-lymphocyte |
| 949 | CD4 T-lymphocyte, CD8 T-lymphocyte |
| 953 | CD4 T-lymphocyte, CD8 T-lymphocyte |
| 958 | CD4 T-lymphocyte, CD8 T-lymphocyte |
| 959 | CD4 T-lymphocyte, CD8 T-lymphocyte |
| 962 | CD4 T-lymphocyte, CD8 T-lymphocyte |
| 966 | CD4 T-lymphocyte, CD8 T-lymphocyte |
| 973 | CD4 T-lymphocyte, CD8 T-lymphocyte |
| 985 | CD4 T-lymphocyte, CD8 T-lymphocyte |
| 986 | CD4 T-lymphocyte, CD8 T-lymphocyte |
| 988 | CD4 T-lymphocyte, CD8 T-lymphocyte |
| 989 | CD4 T-lymphocyte, CD8 T-lymphocyte |
| 993 | CD4 T-lymphocyte, CD8 T-lymphocyte |
| 997 | CD4 T-lymphocyte, CD8 T-lymphocyte |
| 1005 | CD4 T-lymphocyte, CD8 T-lymphocyte |
| 1019 | CD4 T-lymphocyte, CD8 T-lymphocyte |
| 1028 | CD4 T-lymphocyte, CD8 T-lymphocyte |
| 1029 | CD4 T-lymphocyte, CD8 T-lymphocyte |
| 1038 | CD4 T-lymphocyte, CD8 T-lymphocyte |
| 1063 | CD4 T-lymphocyte, CD8 T-lymphocyte |
| 1070 | CD4 T-lymphocyte, CD8 T-lymphocyte |
| 1082 | CD4 T-lymphocyte, CD8 T-lymphocyte |
| 1090 | CD4 T-lymphocyte, CD8 T-lymphocyte |
| 1100 | CD4 T-lymphocyte, CD8 T-lymphocyte |
| 1106 | CD4 T-lymphocyte, CD8 T-lymphocyte |
| 1107 | CD4 T-lymphocyte, CD8 T-lymphocyte |
| 1113 | CD4 T-lymphocyte, CD8 T-lymphocyte |
| 1114 | CD4 T-lymphocyte, CD8 T-lymphocyte |
| 1116 | CD4 T-lymphocyte, CD8 T-lymphocyte |
| 1122 | CD4 T-lymphocyte, CD8 T-lymphocyte |
| 1126 | CD4 T-lymphocyte, CD8 T-lymphocyte |
| 1128 | CD4 T-lymphocyte, CD8 T-lymphocyte |
| 1141 | CD4 T-lymphocyte, CD8 T-lymphocyte |
| 894 | CD4 T-lymphocyte, CD8 T-lymphocyte |
| 896 | CD4 T-lymphocyte, CD8 T-lymphocyte |
| 1110 | CD4 T-lymphocyte, CD8 T-lymphocyte |
| 911 | CD4 T-lymphocyte, CD8 T-lymphocyte |
| 1132 | CD4 T-lymphocyte, CD8 T-lymphocyte |
| 1137 | CD8 T-lymphocyte |
| 853 | fibroblast |
| 871 | fibroblast |
| 877 | fibroblast |
| 904 | fibroblast |
| 935 | fibroblast |
| 955 | fibroblast |
| 965 | fibroblast |
| 994 | fibroblast |
| 998 | fibroblast |
| 1000 | fibroblast |
| 1011 | fibroblast |
| 1015 | fibroblast |
| 1017 | fibroblast |
| 1025 | fibroblast |
| 1032 | fibroblast |
| 1041 | fibroblast |
| 1042 | fibroblast |
| 1048 | fibroblast |
| 1057 | fibroblast |
| 1061 | fibroblast |
| 1062 | fibroblast |
| 1067 | fibroblast |
| 1069 | fibroblast |
| 1072 | fibroblast |
| 1073 | fibroblast |
| 1074 | fibroblast |
| 1076 | fibroblast |
| 1077 | fibroblast |
| 1078 | fibroblast |
| 1079 | fibroblast |
| 1084 | fibroblast |
| 1086 | fibroblast |
| 1091 | fibroblast |
| 1119 | fibroblast |
| 1121 | fibroblast |
| 1130 | fibroblast |
| 1139 | fibroblast |
| 1140 | fibroblast |
| 902 | fibroblast |
| 1003 | fibroblast |
| 1071 | fibroblast |
| 1007 | fibroblast |
| 861 | heart muscle |
| 1010 | heart muscle |
| 1026 | heart muscle |
| 1046 | heart muscle |
| 1050 | heart muscle |
| 1129 | heart muscle |
| 1131 | heart muscle |
| 855 | heart muscle |
| 956 | differentiation between heart muscle and skeletal muscle |
| 1021 | differentiation between heart muscle and skeletal muscle |
| 1030 | differentiation between heart muscle and skeletal muscle |
| 1135 | differentiation between heart muscle and skeletal muscle |
| 894 | keratinocyte |
| 864 | keratinocyte |
| 866 | keratinocyte |
| 870 | keratinocyte |
| 878 | keratinocyte |
| 881 | keratinocyte |
| 885 | keratinocyte |
| 891 | keratinocyte |
| 892 | keratinocyte |
| 893 | keratinocyte |
| 925 | keratinocyte |
| 926 | keratinocyte |
| 930 | keratinocyte |
| 932 | keratinocyte |
| 937 | keratinocyte |
| 943 | keratinocyte |
| 947 | keratinocyte |
| 951 | keratinocyte |
| 952 | keratinocyte |
| 957 | keratinocyte |
| 963 | keratinocyte |
| 964 | keratinocyte |
| 967 | keratinocyte |
| 970 | keratinocyte |
| 972 | keratinocyte |
| 980 | keratinocyte |
| 981 | keratinocyte |
| 982 | keratinocyte |
| 987 | keratinocyte |
| 990 | keratinocyte |
| 992 | keratinocyte |
| 995 | keratinocyte |
| 996 | keratinocyte |
| 1001 | keratinocyte |
| 1002 | keratinocyte |
| 1006 | keratinocyte |
| 1018 | keratinocyte |
| 1020 | keratinocyte |
| 1023 | keratinocyte |
| 1031 | keratinocyte |
| 1033 | keratinocyte |
| 1034 | keratinocyte |
| 1035 | keratinocyte |
| 1036 | keratinocyte |
| 1039 | keratinocyte |
| 1040 | keratinocyte |
| 1045 | keratinocyte |
| 1056 | keratinocyte |
| 1058 | keratinocyte |
| 1059 | keratinocyte |
| 1064 | keratinocyte |
| 1066 | keratinocyte |
| 1080 | keratinocyte |
| 1081 | keratinocyte |
| 1093 | keratinocyte |
| 1094 | keratinocyte |
| 1097 | keratinocyte |
| 1098 | keratinocyte |
| 1101 | keratinocyte |
| 1108 | keratinocyte |
| 1118 | keratinocyte |
| 1120 | keratinocyte |
| 1123 | keratinocyte |
| 1127 | keratinocyte |
| 1133 | keratinocyte |
| 1134 | keratinocyte |
| 1138 | keratinocyte |
| 1140 | keratinocyte |
| 902 | keratinocyte |
| 1003 | keratinocyte |
| 1071 | keratinocyte |
| 1007 | keratinocyte |
| 1044 | keratinocyte |
| 846 | liver |
| 858 | liver |
| 865 | liver |
| 879 | liver |
| 887 | liver |
| 888 | liver |
| 934 | liver |
| 939 | liver |
| 960 | liver |
| 968 | liver |
| 971 | liver |
| 977 | liver |
| 979 | liver |
| 984 | liver |
| 999 | liver |
| 1013 | liver |
| 1014 | liver |
| 1022 | liver |
| 1037 | liver |
| 1047 | liver |
| 1051 | liver |
| 1092 | liver |
| 1111 | liver |
| 1115 | liver |
| 1124 | liver |
| 1136 | liver |
| 1142 | liver |
| 1132 | liver |
| 1044 | liver |
| 936 | liver |
| 849 | melanocyte |
| 854 | melanocyte |
| 874 | melanocyte |
| 886 | melanocyte |
| 909 | melanocyte |
| 918 | melanocyte |
| 919 | melanocyte |
| 927 | melanocyte |
| 954 | melanocyte |
| 976 | melanocyte |
| 1049 | melanocyte |
| 1075 | melanocyte |
| 1087 | melanocyte |
| 1102 | melanocyte |
| 1103 | melanocyte |
| 1105 | melanocyte |
| 1112 | melanocyte |
| 902 | melanocyte |
| 1003 | melanocyte |
| 1071 | melanocyte |
| 1007 | melanocyte |
| 863 | skeletal muscle |
| 884 | skeletal muscle |
| 897 | skeletal muscle |
| 900 | skeletal muscle |
| 903 | skeletal muscle |
| 929 | skeletal muscle |
| 931 | skeletal muscle |
| 945 | skeletal muscle |
| 948 | skeletal muscle |
| 961 | skeletal muscle |
| 975 | skeletal muscle |
| 978 | skeletal muscle |
| 1004 | skeletal muscle |
| 1008 | skeletal muscle |
| 1016 | skeletal muscle |
| 1053 | skeletal muscle |
| 1088 | skeletal muscle |
| 1095 | skeletal muscle |
| 1099 | skeletal muscle |
| 1104 | skeletal muscle |
| 1117 | skeletal muscle |
| 872 | skeletal muscle |
| 855 | skeletal muscle |
| 933 | skeletal muscle |
| 950 | skeletal muscle |
| 1060 | skeletal muscle |
| 851 | skeletal muscle |
| 1043 | skeletal muscle |
| 1052 | skeletal muscle |
| 1055 | skeletal muscle |
| 1109 | skeletal muscle |
| 1089 | skeletal muscle |

### EXAMPLES

### Example 1

### Expression Analysis of Cell- and Tissue Markers According to the Invention

According to the present invention, the methylation status of particular regions of certain genes (as disclosed in Table 2) were found to have differential expression levels and methylation patterns that were consistent within each cell type.

The analysis procedure was as follows. Genes were chosen for analysis based on suspected relevance to particular cell types or cell states according to scientific literature. In general, the candidates were selected from conventional markers for specific cell types, those showing strong or consistently differential expression patterns, housekeeping genes or genes associated with diseases in particular tissues (see literature as cited above regarding cell- and tissue markers). Alternatively, candidate genes can be identified by discovery methods, such as MCA.

Generally, two PCR amplicons (200-500 base pairs long) were designed for each gene, but mainly due to the low complexity of bisulfite-treated DNA and the requirement to avoid CpG sites within the primer (which may or may not be methylated), primers for only approximately 250 amplicons were designed and created.

In most cases, DNA from at least three independent samples (representing standard examples of the cell types as might be obtained routinely by purchase, biopsy, etc.) for each known cell type were isolated using the Qiagen DNeasy Tissue Kit (catalog number 69504), according to the protocol “Purification of total DNA from cultivated animal cells”. This DNA was treated with bisulfite and amplified using primers as designed above.

The amplicons from each gene from each cell type were bisulfite sequenced (Frommer et al., Proc Natl Acad Sci USA 89:1827-1831, 1992). The raw sequencing data was analysed with a program that normalises sequencing traces to account for the abnormal lack of C signal (due to bisulfite conversion of all unmethylated C's) and for the efficiency of the bisulfite treatment (Lewin et al., Bioinformatics 20:3005-12, 2004).

A gene was regarded as relevant, if at least 1 CpG site showed significant distinctions between some pair of cell types, as for the present purposes, a single distinctive CpG within each gene is sufficient to serve as a marker. The statistical significance was generally determined by the Fisher criteria, which compares the variation between classes (i.e., different cell types) versus the variation within a class (i.e., one cell type).

While all of these markers carry useful information in various contexts, there are several subclasses with potentially variable utility. For example, certain genes will show large blocks of consecutive CpGs which are either strongly methylated or strongly unmethylated in many cell types. Because of their ‘all-or-none’ character, these markers are likely to be very consistent and easy to interpret for many cell types. In other cases, the discriminatory methylation may be restricted to one or a few CpGs within the gene, but these individual CpGs can still be reliably assayed, as with single base extension. In addition to markers that show absolute patterns (i.e., nearly 0% or 100% methylation), markers/CpGs that are consistently, e.g., 30% methylated in one cell type and 70% methylated in another cell type are also very useful. Table 3 provides an overview of the characteristic methylation ranges of a selection of the identified, and preferred markers.

The markers as described and preferred, for example, in Table 2 therefore represent epigenetically sensitive markers that are then capable of distinguishing at least one cell and/or tissue type from any other cell and or tissue type.

### Example 2

### Pan-Cancer Method for Diagnosis and or Screening of Cancers

The following example provides a method for the diagnosis of cancer by analysis of the methylation patterns of a panel of genes consisting of the (general) cell proliferation markers SEQ ID NO: 109 and SEQ ID NO: 103 and the tissue- and/or cell-specific markers SEQ ID NO: 80, SEQ ID NO: 76, SEQ ID NO: 57, SEQ ID NO: 84 and SEQ ID NO: 58, as listed in Tables 1 and 2. DNA isolation and bisulfite conversion.

A blood sample is taken from the subject. DNA is isolated from the sample by means of the Magna Pure method (Roche) according to the manufacturer's instructions. The eluate resulting from the purification is then converted according to the following bisulfite reaction. The eluate is mixed with 354 μl of bisulfite solution (5.89 mol/l) and 146 μl of dioxane comprising a radical scavenger (6-hydroxy-2,5,7,8-tetramethylchromane 2-carboxylic acid, 98.6 mg in 2.5 ml of dioxane). The reaction mixture is denatured for 3 min at 99° C. and subsequently incubated at the following temperature program for a total of 7 h min 50° C.; one thermospike (99.9° C.) for 3 min; 1.5 h 50° C.; one thermospike (99° C.) for 3 min; 3 h 50° C. The reaction mixture is subsequently purified by ultrafiltration using a Millipore Microcon™ column. The purification is conducted essentially according to the manufacturer's instructions. For this purpose, the reaction mixture is mixed with 300 μl of water, loaded onto the ultrafiltration membrane, centrifuged for 15 min and subsequently washed with 1×TE buffer. The DNA remains on the membrane during this treatment. Then desulfonation is performed. For this purpose, 0.2 mol/l NaOH is added and incubated for 10 min. A centrifugation (10 min) is then conducted, followed by a washing step with 1×TE buffer. After this, the DNA is eluted. For this purpose, the membrane is mixed for 10 minutes with 75 μl of warm 1×TE buffer (50° C.). The membrane is turned over according to the manufacturer's instructions. Subsequently a repeated centrifugation is conducted, whereby the DNA is removed from the membrane. 10 μl of the eluate is utilized for further analysis.

### Quantitative Methylation Assay

A suitable assay for measurement of the methylation of the target genes is the quantitative methylation (QM) assay. The bisulfite treated DNA is amplified in a PCR reaction using primers specific to bisulfite treated DNA (i.e. each hybridising to at least one thymine position that is a bisulfite converted unmethylated cytosine). The amplification is carried out in the presence of two species of probes, each hybridising to the same target sequence said target sequence comprising at least one cytosine position (pre-bisulfite treatment) wherein one species is specific for the bisulfite converted unmethylated variant of the target sequence (i.e. comprises one or more TG dinucleotides) and the other species is specific for the bisulfite converted methylated variant (i.e. comprises one or more CG dinucleotides). Each species is alternatively detectably labelled, preferably by means of fluorescent labels such as HEX, FAM and VIC and a quencher (e.g. black hole quencher). Hybridisation of the probes to the amplificate is detected by monitoring of the fluorescent labels. Primers and probes for the amplification and analysis of the regions of interest are shown below.

SEQ ID NO: 84
(SEQ ID NO: 806)
Forward primer:
ctacaacaaaatactccaattattaaaac
(SEQ ID NO: 807)
Reverse primer:
gggttaattttgtagaattgtaggt
(SEQ ID NO: 808)
CG probe:
cgtaaaccgtactccaaaatcccga
(SEQ ID NO: 809)
TG probe:
cataaaccatactccaaaatcccaacctc
Amplificate:
(SEQ ID NO: 810)
ctacaacaaaatactccaattattaaaactcatcacgtaaaccgtactccaaaatcccgacctcttcgtaaacatacctacaattctacaaa
attaaccc
Genomic equivalent:
(SEQ ID NO: 811)
ctgcagcaaggtgctccaattgttgaaactcatcacgtgggccgtgctccagagtcceggcctcttcgtggacatgcctgcaattctgca
ggattgaccc
SEQ ID NO: 84
(SEQ ID NO: 812)
Forward primer:
aaaccaacctaaccaatataataaaac
(SEQ ID NO: 813)
Reverse primer:
ggatttaagtgatttttttgttttagt
(SEQ ID NO: 814)
CG probe:
caaccgaatataataacgaacgcctataat
(SEQ ID NO: 815)
TG probe:
caaccaaatataataacaaacacctataatcca
Amplificate:
(SEQ ID NO: 816)
Aaaccaacctaaccaatataataaaaccccgtctctactaaaaatacaaaaatcaaccgaatataataacgaacgcctataatcccaatt
actcgaaaaactaaaacaaaaaaatcacttaaatcc
Genomic equivalent:
(SEQ ID NO: 817)
Agaccagcctggccaatgtagtgaaaccccgtctctactaaaaatacaaaaatcagccgggtatggtggcgggcgcctgtaatccca
gttactcgggaggctgaggcaggagaatcacttgaatcc
SEQ ID NO: 57
(SEQ ID NO: 818)
Forward primer:
cacaatatttcactttaataatattaaaaac
(SEQ ID NO: 819)
CG probe:
aataataaaacgaaaacctcgataacgattaa
(SEQ ID NO: 820)
TG probe:
aataataaaacaaaaacctcaataacaattaaaaaaactata
(SEQ ID NO: 821)
Reverse primer:
tttaaattattgtttaagatttggataaag
Amplificate:
(SEQ ID NO: 822)
cacaatatttcactttaataatattaaaaaccgatacaatcaaaaccaccacaataataaaacgaaaacctcgataacgattaaaaaaacta
taaatctttcgctttatccaaatcttaaacaataatttaaa
Genomic equivalent:
(SEQ ID NO: 823)
cacagtatttcactttaataatattggaaaccggtacagtcagggccaccacagtggtggggcgggagcctcgatggcgattagggga
gctgtaagtctttcgctttatccaaatcttgggcagtaatttaga
SEQ ID NO: 76
(SEQ ID NO: 824)
CG probe:
cgtaaccatattaaacgcaaataaacgc
(SEQ ID NO: 825)
Forward primer:
aaatcaaaataaacacaattaaaaaca
(SEQ ID NO: 826)
TG probe:
cataaccatattaaacacaaataaacacaataacaaaa
(SEQ ID NO: 827)
Reverse primer:
aattgagaagtaaaatagtttagtttattagag
Amplificate:
(SEQ ID NO: 828)
aaatcaaaataaacacaattaaaaacattaaaccgtaaccatattaaacgcaaataaacgcaataacaaaattctttaaactctaataaact
aaactattttacttctcaatt
Genomic equivalent:
(SEQ ID NO: 829)
aaatcaaaataggcacagttgggaacattaagccgtggccatattagacgcaagtaggcgcaatagcaaaattctttaggctctaatgg
actgggctattttgcttctcagtt
SEQ ID NO: 80
(SEQ ID NO: 830)
Forward primer:
ctataaaaccaacaaaaaatatttcaa
(SEQ ID NO: 831)
CG probe:
aattttattacgccaacgcgactataaattaa
(SEQ ID NO: 832)
TG probe:
aattttattacaccaacacaactataaattaaaaaaacatct
(SEQ ID NO: 833)
Reverse primer:
aaaattggtatttattttggtttatatg
Amplificate:
(SEQ ID NO: 834)
ctataaaaccaacaaaaaatatttcaaaccatcgaaattttattacgccaacgcgactataaattaaaaaaacatctccatataaaccaaaa
taaataccaatttt
Genomic equivalent:
(SEQ ID NO: 835)
gctgtgaagccagcaaaaggtatttcaggccatcgaagttttgttgcgccagcgcggctgtagattagaaggacatctccatgtgaacc
aagatggatgccaatttt
SEQ ID NO: 103
(SEQ ID NO: 836)
Forward primer:
tagggtaggttggtttgtgttg
(SEQ ID NO: 837)
Reverse primer:
ctttccctacctccttaaataactacc
(SEQ ID NO: 838)
CG probe:
cgcgtgtttttttgcggagtta
(SEQ ID NO: 839)
TG probe:
atgtgtgtttvtttgtggagttaaag
SEQ ID NO: 109
(SEQ ID NO: 840)
Forward primer:
aacaaccaaaactaaaaaccaaaact
(SEQ ID NO: 841)
Reverse primer:
tagtgaagaatggtgttggatttt
(SEQ ID NO: 842)
TG probe:
cacaccacctacacacacaacctcac
(SEQ ID NO: 843)
CG probe:
cgcgccacctacgc

For each assay, the amount of amplificate detected by each probe species is quantified by reference to a standard curve. The standard curve is plotted by measuring the Ct of a series of bisulfite converted DNA solutions of known degrees of methylation assayed using the respective assay. Preferably the Ct of a series of bisulfite converted genomic DNAs of 0, 5, 10, 25, 50, 75 and 100% methylation is determined. The DNA solutions may be prepared by mixing known quantities of completely methylated and completely unmethylated genomic DNA. Completely unmethylated genomic DNA is available from commercial suppliers such as but not limited to Molecular Staging, and may be prepared by a multiple displacement amplification of human genomic DNA (e.g. from whole blood). Completely methylated DNA may be prepared by SssI treatment of a genomic DNA sample, preferably according to manufacturer's instructions. Bisulfite conversion may be carried out as described above.

The real-time PCR is carried out using commercially available real time PCR instruments e.g. ABI7700 Sequence Detection System (Applied Biosystems), in a 20 μl reaction volume. Using said instrument a suitable reaction solution is:

1× TaqMan Buffer A (Applied Biosystems) containing ROX as a passive reference dye
2.5 mmol/l MgCl₂ (Applied Biosystems)
1 U of AmpliTaq Gold DNA polymerase (Applied Biosystems)
625 nmol/l primers
200 nmol/l probes
200 μmol/l dNTPs

### Temperature Cycling Profile:

Initial 10 min activation at 94° C. followed by 45 cycles of 15 s at 94° C. (for denaturation) and 60 s at 60° C. (for annealing, elongation and detection).

Data analysis is preferably conducted according to the instrument manufacturer's recommendations. The degree of methylation is determined according to the following formula:

methylation rate=delta Rn CG probe/(delta Rn CG probe+delta Rn TG probe)

Alternatively, the methylation rate may be determined according to the threshold cycles (Ct), wherein

methylation rate=100/(1+2^{delta Ct})

A detected methylation rate of over 4% is determined to be methylated.

The presence, absence and type of cell proliferative disorder is then determined by reference to Tables 1 and 2, wherein methylation of either of the genes according to SEQ ID NO: 103 and SEQ ID NO: 109 is indicative of the presence of cell proliferative disorders. Wherein the presence of methylation of said genes is determined, methylation of the further genes is determined in order to localize the cell proliferative disorder.

The presence of unmethylated SEQ ID NO: 80 DNA is indicative of soft tissue sarcoma. The presence of unmethylated SEQ ID NO: 76 DNA is indicative of the presence of a melanoma. The presence of unmethylated SEQ ID NO: 57 DNA is indicative of abnormal keratinocyte proliferation e.g. psoriasis. The presence of unmethylated SEQ ID NO: 84 DNA is indicative of liver cancer. The presence of unmethylated SEQ ID NO: 58 DNA is indicative of soft tissue sarcoma.

1. Method for diagnosing a proliferative disease in a subject comprising:
a) providing a biological sample from a subject,
b) detecting the presence, absence, abundance and/or expression of one or more markers and determining therefrom upon the presence or absence of a proliferative disease; and
c) detecting the presence, absence, abundance and/or expression of one or more cell- and/or tissue-markers and determining therefrom if said one or more cell- and/or tissue-markers are atypically present, absent or present at above normal levels within said sample; and
d) determining the presence or absence of a cell proliferative disorder and location thereof based on the presence, absence, abundance and/or expression as detected in step b) and c).

2. The method according to claim 1, further comprising detecting the presence, absence, abundance and/or expression of one or more markers and determining therefrom characteristics of said cell proliferative disorder.

3. The method according to claim 1 or 2, wherein said marker in step b) is indicative of more than one proliferative disease.

4. The method according to any of claims 1 to 3, wherein said proliferative disease is cancer.

5. The method according to any of claims 1 to 4, wherein said detecting the presence, absence, abundance and/or expression of one or more markers comprises detecting physiological, genetic, and/or cellular presence, absence, abundance and/or expression, and cell count.

6. The method according to claim 5, wherein said detecting the expression comprises detecting the expression of protein, mRNA expression and/or the presence or absence of DNA methylation in one or more of said markers.

7. The method according to any of claims 1 to 6, comprising the steps of:
a) providing a biological sample from a subject, said biological sample comprising genomic DNA;
b) detecting the level of DNA methylation in one or more markers and determining therefrom upon the presence or absence of a proliferative disease; and
c) detecting the level of methylation of one or more markers and determining therefrom if said one or more cell- and/or tissue-markers are atypically present, absent or present at above normal levels within said sample; and
d) determining the presence or absence of a cell proliferative disorder and location thereof, based on the level of DNA methylation as detected in step b) and c).

8. The method according to claim 7, wherein the determining the presence or absence of a cell proliferative disorder of step b) further comprises comparing said methylation profile to one or more standard methylation profiles, wherein said standard methylation profiles are selected from the group consisting of methylation profiles of non cell proliferative disorder samples and methylation profiles of cell proliferative disorder samples.

9. The method according to any of claims 1 to 8, wherein the markers of step b) are selected from the group consisting of nucleic acid sequences according to any of SEQ ID NO: 100 to SEQ ID NO: 161.

10. The method according to any of claims 1 to 9, wherein the markers of step c) are selected from the group consisting of nucleic acid sequences according to any of SEQ ID NO: 1 to SEQ ID NO: 99 and SEQ ID NO: 844 to SEQ ID NO: 1255.

11. The method according to any of claims 1 to 10, wherein said characterizing cancer comprises determining the likelihood of disease-free survival, and/or monitoring disease progression in said subject.

12. The method according to any of claims 1 to 10, wherein said characterizing cancer comprises determining metastatic disease.

13. The method according to any of claims 1 to 10, wherein said characterizing cancer comprises determining relapse of the disease after complete resection of the tumor in said subject by identifying tissue markers and cancer markers in said sample that are identical to the removed tumor.

14. The method according to any of claims 1 to 13, wherein said biological sample is a biopsy sample or a blood sample.

15. The method according to any of claims 1 to 14, wherein said proliferative disease is in the early pre-clinical stage exhibiting no clinical symptoms.

16. The method according to any of claims 7 to 15, wherein said detecting the presence or absence of DNA methylation comprises treatment of said genomic DNA with one or more reagents suitable to convert 5-position unmethylated cytosine bases to uracil or to another base that is detectably dissimilar to cytosine in terms of hybridization properties.

17. The method according to claim 16, wherein the markers of step b) are selected from the group consisting of nucleic acid sequences according to any of SEQ ID NO: 100 to SEQ ID NO: 161, and SEQ ID NO: 360 to SEQ ID NO: 483, and SEQ ID NO: 682 to SEQ ID NO: 805.

18. The method according to claim 16 or 17, wherein said the markers of step c) are selected from the group consisting of nucleic acid sequences according to any of SEQ ID NO: 1 to SEQ ID NO: 99, and SEQ ID NO: 162 to SEQ ID NO: 359, and SEQ ID NO: 484 to SEQ ID NO: 681 and SEQ ID NO: 844 to SEQ ID NO: 2903.

19. Method for generating a pan-cancer marker panel for the improved diagnosis and/or monitoring of a proliferative disease in a subject, comprising
a) providing a biological sample from said subject suspected of or previously being diagnosed as having a proliferative disease,
b) providing a first set of one or more markers indicative for proliferative disease,
c) determining the presence, absence, abundance and/or expression of said one or more markers of step b);
d) providing a first set of cell- and/or tissue markers,
e) determining the expression of said one or more markers of step d), and
f) generating a pan-cancer marker panel that is specific for said proliferative disease in said subject by selecting those markers that are differently expressed in said subject when compared to an expression profile of a healthy sample.

20. The method according to claim 19, wherein said detecting the presence, absence, abundance and/or expression of one or more markers comprises detecting physiological, genetic, and/or cellular presence, absence, abundance and/or expression, and cell count, measuring the expression of protein, mRNA expression and/or the presence or absence of DNA methylation in one or more of said markers.

21. The method according to claim 19 or 20, wherein said marker is indicative of more than one proliferative disease.

22. The method according to any of claims 19 to 21, wherein the markers of step b) are selected from the group consisting of nucleic acid sequences according to any of SEQ ID NO: 100 to SEQ ID NO: 161.

23. The method according to any of claims 19 to 22, wherein the markers of step c) are selected from the group consisting of nucleic acid sequences according to any of SEQ ID NO: 1 to SEQ ID NO: 99 SEQ ID NO: 844 to SEQ ID NO: 1255.

24. The method according to any of claims 19 to 23, wherein said proliferative disease is selected from cancer, such as soft tissue, skin, leukemia, renal, prostate, brain, bone, blood, lymphoid, stomach, head and neck, colon or breast cancer.

25. The method according to any of claims 19 to 24, wherein said proliferative disease is in the early pre-clinical stage exhibiting no clinical symptoms.

26. The method according to any of claims 1 to 25, wherein said detecting of the expression is qualitative or additionally quantitative.

27. An improved method for treatment of a proliferative disease, comprising a method according to any of claims 1 to 26 and selecting a suitable treatment regimen for said proliferative disease to be treated.

28. The method according to claim 27, wherein said proliferative disease is cancer.

29. A kit for diagnosing a proliferative disease in a subject, comprising reagents for detecting the expression of one or more marker indicative for more than one proliferative disease; and reagents for localizing the proliferative disease and/or characterizing the type of proliferative disease by detecting specific tissue markers based on nucleic acid-analysis.

30. Kit according to claim 29, wherein the markers are selected from the group consisting of nucleic acid sequences according to any of SEQ ID NO: 1 to SEQ ID NO: 161 and SEQ ID NO: 844 to SEQ ID NO: 1255, and chemically pretreated sequences thereof.

31. Kit according to claim 29 or 30, further containing instructions for using said kit for detecting of a proliferative disease, in particular cancer, in said subject.
